(19) United States
(12) Patent Application Publication
(10) Pub. No.: US 2026/0348955 A1
(43) Pub. Date: Nov. 5, 2026

(54) AUTOANTIBODIES AS MARKERS OF PRETERM BIRTH

(71) Applicants: CZ Biohub SF, LLC, San Francisco, CA (US); The Regents of the University of California, Oakland, CA (US)

(72) Inventors: Elze Rackaityte, Berkeley, CA (US); Joseph L. DeRisi, San Francisco, CA (US)

(73) Assignees: CZ Biohub SF, LLC, San Francisco, CA (US); The Regents of the University of California, Oakland, CA (US)

(21) Appl. No.: 19/486,116

(22) PCT Filed: May 28, 2024
(86) PCT No.: PCT/US2024/031250

§ 371 Date: Nov. 20, 2025

Related U.S. Application Data
(60) Provisional application No. 63/504,626, filed on May 26, 2023.
(60) Provisional application No. 63/564,699, filed on Mar. 13, 2024.

(51) Int. Cl. G01N 33/564; G01N 33/53; G01N 33/543; G01N 33/68

(52) U.S. Cl. CPC G01N 33/564; G01N 33/5306; G01N 33/543; G01N 33/6869; G01N 33/689; G01N 2333/545; G01N 2474/10; G01N 2800/368; G01N 2800/50; G01N 2800/52

(57) ABSTRACT

Provided herein are methods of detecting autoantibodies associated with a risk of premature labor and methods of identifying subjects at risk of premature labor by detecting in biological samples obtained from the subjects the autoantibodies associated with the risk of premature labor.

### BACKGROUND

Immunological tolerance of the semi-allogeneic fetus is critical for the maintenance of pregnancy and breakthrough of tolerance is associated with preterm birth, the leading cause of death in children worldwide. Despite the importance of this process and the broader implications for understanding of immunity, relatively little is known about the antigen-specific adaptive immune responses to the human fetus. Naturally occurring antibodies in pregnancy exhibit varied phenotypes and, in some cases, contribute to inflammation-related pregnancy pathologies. For example, antibodies against Rhesus antigen (RhD) are generated in RhD-negative mothers bearing an RhD-positive fetus and cause hemolytic disease of the fetus and newborn. Autoantibodies can also contribute to pregnancy pathologies such as in systemic lupus erythematosus patients with anti-phospholipid antibodies who have high rates of miscarriage attributed to antibody recognition of placental antigens and complement deposition on placental trophoblasts. Not all antibodies generated during pregnancy threaten the fetus. For example, antibodies with specificities to paternal HLA molecules are commonly found in healthy pregnancies and are not associated with pregnancy complications. Post-partum however, these anti-HLA antibodies are capable of complicating organ transplantation in the mother and the paternal genotype must be considered prior to organ donor selection. The distinction between putatively pathogenic and benign antibody reactivities during pregnancy have not been sufficiently investigated. In view of the importance of early detection of those at high risk for preterm birth, it is important to identify and detect associated antibodies.

### BRIEF SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention are methods of detecting an anti-interleukin-1 receptor antagonist (IL1RA) antibody in a biological sample, such methods comprising the steps of contacting the biological sample with an IL1RA polypeptide, and detecting binding of the IL1RA polypeptide to the anti-IL1RA antibody in the biological sample. Also included among the embodiments of the present invention are methods of identifying a subject at risk of premature labor, such methods comprising the steps of contacting the biological sample obtained from the subject with an IL1RA polypeptide, and detecting presence of binding of the IL1RA polypeptide to the anti-IL1RA antibody in the biological sample, wherein the presence of binding of the IL1RA polypeptide to the anti-IL1RA antibody in the biological sample identifies the subject at risk of premature labor. In some embodiments of the above methods, the subject may be a pregnant subject. Also included among the embodiments of the present invention are methods of detecting presence of an anti-IL1RA antibody in a pregnant subject and treating the pregnant subject, such methods comprising the steps of contacting a biological sample obtained from the pregnant subject with an IL1RA polypeptide, detecting presence of binding of the IL1RA polypeptide to the anti-IL IRA antibody in the biological sample, and administering to the pregnant subject one or more treatments intended to prevent premature labor or to promote fetal maturation. The one or more treatments intended to prevent premature labor or to promote fetal maturation may comprise one or more of putting the pregnant subject on bedrest, administering to the pregnant subject one or more tocolytic medication, administering to the pregnant subject one or more corticosteroid, performing cervical cerclage on the pregnant subject. Also included among the embodiments of the present invention are methods of detecting presence of an anti-IL1RA antibody in a pregnant subject and monitoring the pregnant subject, such methods comprising: the steps of contacting a biological sample from the pregnant subject with an IL1RA polypeptide, detecting presence of binding of the IL1RA polypeptide to the anti-IL1RA antibody in the biological sample, and performing on the pregnant subject one or more procedures intended to monitor labor or state of the fetus. The one or more procedures intended to monitor labor or state of the fetus may comprise one or more of monitoring contractions, monitoring fetal heart rate, performing one or more cervical exams, performing transvaginal ultrasound exam, testing cervical or vaginal sample obtained from the pregnant subject for amniotic fluid, testing cervical or vaginal sample obtained from the pregnant subject for fetal fibronectin. Also included among the embodiments of the present invention are methods of detecting presence of an anti-IL1RA antibody in a subject and treating the subject (which may be a pregnant subject), such methods comprising the steps of contacting a biological sample from the subject with an IL1RA polypeptide, detecting presence of binding of the IL1RA polypeptide to the anti-IL1RA antibody in the biological sample, and administering to the subject an immunosuppressive treatment and/or a treatment reducing amount of the anti-IL1RA antibody in the subject.

Also included among the embodiments of the present invention are method of treating a subject harboring anti-IL1RA antibody. In some embodiments, such methods comprise administering to the subject one or more of: an immunosuppressive treatment; a treatment reducing amount of the anti-IL1RA antibody in the subject; a therapeutically effective amount of IL1RA or an IL1RA analog; or a therapeutically effective amount of an IL1RA antagonist. In some embodiments the IL1RA antagonist is anakinra.

In the above methods according to the exemplary embodiments of the present invention, the IL1RA polypeptide may be a heterologously expressed IL1RA polypeptide. The heterologously expressed IL1RA polypeptide may be expressed on the surface of a cell, on a phage, or on a virus. The heterologously expressed IL1RA polypeptide may expressed in a phage display or eukaryotic cell display library. The IL1RA polypeptide may be an isolated, polypeptide. The IL1RA polypeptide may be immobilized on a solid carrier. The IL1RA polypeptide may comprise a sequence with at least 90% sequence similarity to SEQ ID NO:2 (LQLEAVNITDLSENRK). In the above exemplary methods according to the embodiments of the present invention, the step of detecting the presence of binding of the IL1RA polypeptide to the anti-IL1RA antibody in the biological sample may comprise performing at least one of immunoprecipitation, microarray analysis, enzyme-linked immunosorbent assay (ELISA), or Western blot analysis. In the above exemplary methods according to the embodiments of the present invention, the biological sample may be serum, plasma, blood, amniotic fluid, or urine. In the above exemplary methods according to the embodiments of the present invention, the subject or the pregnant subject may be human. The pregnant subject may be a primigravid or a multigravida. The subject or the pregnant subject may have previously experienced premature labor or premature delivery. In the above exemplary methods according to the embodiments of the present invention, the anti-IL1RA antibody may be an alloantibody or an autoantibody.

Also included among the embodiments of the present invention are kits for detecting anti-IL1RA antibodies in a sample. Such kits may comprise one or more IL1RA polypeptides and one or more other reagents for detecting the anti-IL1RA antibodies in the sample. The at least one of the one or more IL1RA polypeptides may comprise a sequence with at least 90% sequence similarity to SEQ ID NO:2. Also included among the embodiments of the present invention are devices for detecting anti-IL1RA antibodies in a sample, such devices comprising one or more IL1RA polypeptides. The least one of the one or more IL1RA polypeptides may comprise a sequence with at least 90% sequence similarity to SEQ ID NO:2. 26. In the devices according to embodiments of the present invention, the one or more IL1RA polypeptides may be immobilized on a surface of a solid carrier included in the device. The solid carrier may be a slide, a chip, a plate, a plurality of fibers, a plurality of beads, a chromatography column, or a membrane. Also included among the embodiments of the present invention are systems comprising a device according to exemplary embodiments of the present invention and one or more reagents, other than the one or more IL1RA polypeptides, for detecting the anti-IL1RA antibodies in the sample.

Also included among the embodiments of the present invention are methods of detecting, in a biological sample, one or more antibodies associated with a risk of premature labor, such methods comprising the steps of: (a) contacting the biological sample with one or more polypeptides comprising a sequence with at least 90% sequence similarity to at least one of SEQ ID NO:3 to SEQ ID NO:631; and (b) detecting binding in the biological sample of the one or more polypeptides to the one or more antibodies associated with a risk of premature labor. It is to be understood that, as used here and throughout the present disclosure, the sequence with at least 90% sequence similarity to the at least one of SEQ ID NO:3 to SEQ ID NO:631 can be a sequence with at least 90%, at least 91%, at least 92%, at least 93%, at least 94%, at least 95%, at least 96%, at least 97%, at least 98%, at least 99%, or 100% sequence similarity to the at least one of SEQ ID NO:3 to SEQ ID NO:631. Also included among the embodiments of the present invention are methods of identifying a subject at risk of premature labor, such methods comprising the steps of: (a) contacting the biological sample obtained from the subject with one or more polypeptides comprising a sequence with at least 90% sequence similarity to at least one of SEQ ID NO:3 to SEQ ID NO:631; and (b) detecting presence of binding in the biological sample of the one or more polypeptides to one or more antibodies, wherein the presence of binding of the one or more polypeptides to the one or more antibodies identifies the subject at risk of premature labor. In some embodiments of the above methods, the subject is a pregnant subject. Also included among the embodiments of the present invention are methods of detecting, in a pregnant subject, presence of one or more antibodies associated with a risk of premature labor and treating the pregnant subject, such methods comprising the steps of: (a) contacting a biological sample obtained from the pregnant subject with one or more polypeptides comprising a sequence with at least 90% sequence similarity to at least one of SEQ ID NO:3 to SEQ ID NO:631; (b) detecting presence of binding in the biological sample of the one or more polypeptides to the one or more antibodies; and (c) administering to the pregnant subject one or more treatments intended to prevent premature labor or to promote fetal maturation. In the embodiments of the above methods, the one or more treatments may comprise one or more of putting the pregnant subject on bedrest, administering to the pregnant subject one or more tocolytic medication, administering to the pregnant subject one or more corticosteroid, performing cervical cerclage on the pregnant subject, or administering immunosuppressive treatment to the pregnant subject. Also included among the embodiments of the present invention are methods of detecting, in a pregnant subject, presence of one or more antibodies associated with a risk of premature labor and monitoring the pregnant subject, such methods comprising the steps of: (a) contacting a biological sample obtained from the pregnant subject with one or more polypeptides comprising a sequence with at least 90% sequence similarity to at least one of SEQ ID NO:3 to SEQ ID NO:631; (b) detecting presence of binding in the biological sample of the one or more polypeptides to the one or more antibodies; and, (c) performing on the pregnant subject one or more procedures intended to monitor labor or state of the fetus. In the embodiments of the above methods, the one or more such procedures may comprise one or more of monitoring contractions, monitoring fetal heart rate, performing one or more cervical exams, performing transvaginal ultrasound exam, testing cervical or vaginal sample obtained from the pregnant subject for amniotic fluid, testing cervical or vaginal sample obtained from the pregnant subject for fetal fibronectin.

In the embodiments of the methods according to the present disclosure and summarized in the previous paragraph, the one or more polypeptides may be heterologously expressed one or more polypeptides. The heterologously expressed one or more polypeptides may be expressed on the surface of a cell, on a phage, or on a virus. The heterologously expressed one or more polypeptides may expressed in a phage display or eukaryotic cell display library. In some embodiments of the methods according to the present disclosure and summarized in the previous paragraph, the one or more polypeptides may be isolated, polypeptides. In some embodiments of the methods according to the present disclosure and summarized in the previous paragraph, the one or more polypeptides may immobilized on a solid carrier. In some embodiments of the methods according to the present disclosure and summarized in the previous paragraph, step (b) (the detecting step) may comprise the step of performing at least one of immunoprecipitation, microarray analysis, enzyme-linked immunosorbent assay (ELISA), or Western blot analysis. In some embodiments of the methods according to the present disclosure and summarized in the previous paragraph, the biological sample may be serum, plasma, blood, amniotic fluid, or urine. In some embodiments of the methods according to the present disclosure and summarized in the previous paragraph, the subject or the pregnant subject is human.

Also included among the embodiments of the present invention are kits for detecting in a sample one or more antibodies associated with a risk of premature labor, comprising one or more polypeptides comprising a sequence with at least 90% sequence similarity to at least one of SEQ ID NO:3 to SEQ ID NO:631 and one or more other reagents for detecting the one or more antibodies in the sample. Also included among the embodiments of the present invention are devices for detecting in a sample one or more antibodies associated with a risk of premature labor, the device comprising one or more polypeptides comprising a sequence with at least 90% sequence similarity to at least one of SEQ ID NO:3 to SEQ ID NO:631. In some embodiments of such devices, the one or more polypeptides may be immobilized on a surface of a solid carrier included in the device. The solid carrier may be is a slide, a chip, a plate, a plurality of fibers, a plurality of beads, a chromatography column, or a membrane. Also included among the embodiments of the present invention are systems comprising the above devices according to the embodiments of the present invention and one or more reagents, other than the one or more polypeptides, for detecting the one or more antibodies in the sample.

Also included among the embodiments of the present invention are methods of detecting antigens of autoantibodies associated with a risk of premature labor, such methods comprising the steps of: (a) contacting a first biological sample obtained from a human subject who has undergone an uncomplicated term pregnancy with a peptide display library under conditions sufficient to permit binding of a first plurality of antibodies from the first biological sample to a first plurality of corresponding peptides within the peptide display library to generate a first plurality of antibody-peptide complexes; (b) contacting a second biological sample obtained from a human subject who has undergone a preterm pregnancy with the peptide display library under conditions sufficient to permit binding of a second plurality of antibodies from the first biological sample to a second plurality of corresponding peptides within the peptide display library to generate a second plurality of antibody-peptide complexes; (c) identifying the first plurality of the peptides and the second plurality of the peptides; and, (d) using a computer system, identifying a third plurality of peptides as the peptides found in the second plurality of the peptides but not in the first plurality of peptides, wherein the third plurality of the peptides are the antigens of the autoantibodies associated with the risk of premature labor. The third plurality of the peptides may comprises peptides with at least 90% sequence similarity to the peptides listed in Table 2 of the present disclosure. In some embodiments of the above methods, the peptide display library nay a phage display library. A nucleic sequence encoding each peptide of such phage display library may a unique nucleic acid barcode sequence. Some embodiments of the above methods may further comprise the steps of: subjecting the first plurality of complexes and the second plurality of complexes to nucleic acid amplification under conditions sufficient to amplify the unique nucleic acid barcode sequence to generate a plurality of amplified nucleic acid sequences; determining sequences of the amplified nucleic acid sequences to generate determined sequences; and, in step (d), using the determined sequences for the identifying the first plurality of the peptides and the second plurality of the peptides.

### BRIEF DESCRIPTION OF THE DRAWINGS

FIG. 1 is a schematic illustration of design of the study according to the present disclosure.

FIG. 2 is a mean receiver operator curve (ROC) performance of five-fold cross-validated lasso-regularized logistic regression model trained on Cohort I and tested on Cohort II (as indicated). Area under the curve (AUC) indicated; shaded region represent=one standard deviation; dashed black line indicates random chance performance.

FIG. 3 is a dot plot illustrating Unsupervised Uniform Manifold Approximation and Projection (UMAP) analysis of placental IP-MS protein counts in Cohort II.

FIG. 4 is a box plot showing the number of preterm-specific autoantibody reactivities in term and preterm pregnancies in Cohort I (left) and Cohort II (right).

FIG. 5 is a box plot illustrating transfer rate of specific autoantibody reactivities in term and preterm dyads.

FIG. 6 are box plots illustrating sum log₁₀ fold-change of over mock-IP of preterm-specific autoreactivities identified in Cohort I in maternal (left) or infant (right) samples in Cohorts III-IV.

FIG. 7 are dot/box plots illustrating the prevalence of anti-IL1RA antibodies in preterm versus term pregnancies, as detected by z-score of fold change above mean in term pregnancies detected by PhIP-seq in Cohort I (left), fold change over mean in term pregnancies detected by placental IP-MS in Cohort II (middle), or normalized optical density (OD450) relative to assay blanks in ELISA utilizing HEK293T IL1RA overexpression lysate as bait for selected patient sera from Cohort II (right).

FIG. 8 are box plots illustrating the results of the quantification of secreted embryonic alkaline phosphatase (SEAP) produced by HEK-Blue IL1B reporter cells incubated with human IL1B and IL1RA in the presence of PBS, off target anti-GFAP, or anti-IL1RA polyclonal antibody (left plot) or male, term, anti-IL1RA⁺ sera or no serum controls (right plot). IL1B activity was calculated by dividing experimental signal with patient sera by cells treated with IL1B alone.

FIG. 9 is a bar graph illustrating the results of quantification of the levels of IL1B (pg/mL) in maternal serum by ELISA.

FIG. 10 is a bar graph illustrating the resorption rate of pups in anti-IL1RA-injected dams as compared to isotype.

FIG. 11 is a bar graph illustrated percentage of malperfused pups among live pups in isotype or anti-IL1RA injected dams at embryonic day (E) 18.5.

FIG. 12 is a bar graph illustrating the difference in fetal weight between normal and malperfused pups.

### DETAILED DESCRIPTION

### Overview

Provided herein are methods of detecting, in a biological sample, antibodies associated with a risk of premature labor, such as an anti-IL1RA antibody, and methods of identifying a subject at risk of premature labor by detecting, in a biological sample obtained from the subject, antibodies associated with a risk of premature labor, such as an anti-IL1RA antibody. An aberrant antibody response is a plausible risk factor for preterm birth because immune dysregulation and inflammation cause preterm labor. Additionally, a previous preterm pregnancy is one of the strongest known predictors of subsequent preterm delivery, indicating an immune memory response contributes to the process. B cells likely play a role in the inflammatory environment of preterm pregnancies: they more readily secrete inflammatory cytokines in mothers who deliver prematurely, and the proportions of regulatory B cells are reduced. In mice, B cell suppression is required for the establishment of feto-maternal tolerance and lack of central immune tolerance during pregnancy results in intrauterine growth restriction, which can be rescued by depletion of T and B cells. This suggests that tight regulation of T and B cells contributes to reproductive success even in highly inbred strains of mice with little to no allogeneic alleles contributing to the process. However, in humans, antibody association with preterm birth has not been previously observed.

The inventors investigated antibody reactivities to the human proteome utilizing phage display immunoprecipitation and sequencing (PhIP-seq) in two separate cohorts of preterm and term pregnancies. In a large, population-wide California cohort, split equally between term and preterm outcomes (n=850), sera at 16 weeks of gestation from preterm pregnancies exhibited a convergence of reactivity that was predictive of future preterm delivery. The majority of PhIP-seq reactivities in preterm sera overlapped with those in maternal sera from a second case-control cohort of preterm premature rupture of membranes (n=55) collected at a median of 25 weeks gestation. Immunoprecipitation-mass spectrometry (IP-MS) of placental lysate and maternal serum was used to identify placenta-targeting antibody reactivities preterm maternal sera. 85% of preterm pregnancy reactivities identified by IP-MS were also found by PhIP-seq, orthogonally validating PhIP-seq findings and suggesting that anti-placental immune responses define subsets of preterm pregnancies.

Among validated hits, which are described elsewhere in the present disclosure, were anti-IL 1 receptor antagonist antibodies (IL1RA) occurring in 20% of preterm and rarely in term pregnancies. IL1B is an inflammatory pyrogen known to rapidly induce labor in non-human primates, and maternal anti-IL1RA antibodies suppressed IL1 antagonism in vitro. Based on the above data, the inventors determined that an anti-IL1RA antibody response in pregnant subjects is an early predictor of preterm delivery and may directly contribute to the inflammatory milieu associated with pregnancy complications by inhibiting anti-inflammatory mediators. Anti-IL1RA antibodies detected in pregnant subjects may be autoantibodies or alloantibodies. In one example, such anti-IL1RA alloantibodies may generated in response to IL1RA originating from the fetus. However, any previous exposure to a non-self IL1RA antigen (for example, during an organ transplant) may trigger alloantibodies. The inventors also conceived that anti-IL1RA antibody response in subjects may precede any pregnancy, may be induced by a prior pregnancy, and/or may persist beyond pregnancy. It is therefore may be advantageous to screen not only pregnant but also non-pregnant subjects contemplating pregnancy for anti-IL1RA antibodies to detect if they are at risk for premature labor.

The inventors also conceived that detection of the autoantibodies to one or more of the peptides listed in Table 2 of the present disclosure may be used for prediction of preterm delivery. Methods of detecting antigens of autoantibodies associated with a risk of premature labor using PhIP-seq are also provided in the present disclosure.

### TERMS AND CONCEPTS

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

As used herein, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used herein “another” can mean at least a second or more.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

The terms “protein,” “peptide,” and “polypeptide” are used interchangeably to refer to a polymer of amino acid residues. The terms apply to naturally occurring amino acid polymers and non-natural amino acid polymers, as well as to amino acid polymers in which one (or more) amino acid residue is an artificial chemical mimetic of a corresponding naturally occurring amino acid. The terms encompass amino acid chains of any length, including full-length proteins, wherein the amino acid residues are linked by covalent peptide bonds.

An “isolated” or “purified” polypeptide or protein, or biologically active portion a polypeptide or a protein, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals. For example, when the IL1RA polypeptide is recombinantly produced, optimally culture medium represents less than about 30%, 20%, 10%, 5%, 1%, 0.5%, or 0.1% (by dry weight) of chemical precursors or non-protein-of-interest chemicals.

The term “amino acid” refers to any monomeric unit that can be incorporated into a peptide, polypeptide, or protein. Amino acids include naturally occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally occurring amino acids are those encoded by the genetic code, as well as those amino acids that are later modified, e.g., hydroxyproline, γ-carboxyglutamate, and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally occurring amino acids. For example, “amino acid analogs” can be unnatural amino acids that have the same basic chemical structure as naturally occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally occurring amino acid. Amino acids may be referred to by either the commonly known three letter symbols or by the one-letter symbols recommended by the IUPAC-IUB Biochemical Nomenclature Commission.

The expression “conservatively modified variant” and related expression may apply to amino acid sequences, as well to nucleic acid sequences encoding amino acid sequence. Substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

The terms “identity,” “substantial identity,” “similarity,” “substantial similarity,” “homology” and the related terms and expressions used in the context of describing nucleic acid or amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of nucleic acid or amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman (Smith and Waterman “Identification of common molecular subsequences.” J Mol Biol. 147 (1): 195-7 (1981)) by the homology alignment algorithm of Needleman and Wunsch (Needleman and Wunsch “A general method applicable to the search for similarities in the amino acid sequence of two proteins.” J Mol Biol. 48 (3): 443-53 (1970)), by the search for similarity method of Pearson and Lipman (Pearson and Lipman “Improved tools for biological sequence comparison.” Proc. Natl. Acad. Sci. USA 85 (8): 2444-8 (1988)), by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al. “Basic local alignment search tool.” J. Mol. Biol. 215:403-410 (1990), and Altschul et al., “Gapped BLAST and PSI-BLAST: a new generation of protein database search programs.” Nucleic Acids Res. 25:3389-3402 (1997), respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, “Amino acid substitution matrices from protein blocks.” Proc. Natl. Acad. Sci. USA 89:10915-10919 (1989)). The BLAST algorithm also performs a statistical analysis of the similarity between two sequences (Karlin and Altschul “Applications and statistics for multiple high-scoring segments in molecular sequences.” Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “antibody” and the related terms refer to an immunoglobulin or its fragment that binds to a particular spatial and polar organization of another molecule. Immunoglobulins include various classes and isotypes, such as IgA, IgD, IgE, IgG1, IgG2a, IgG2b and IgG3, IgG4, IgM, etc., Naturally occurring antibodies are encoded by immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. An antibody can be specific for a particular antigen. The antibody or its antigen can be either an analyte or a binding partner. The term “antibody” encompasses autoantibodies, which are produced by an immune system of an individual against individual's own antigens. For example, a an anti-ILRIA autoantibody is produced by an individual's immune system against individual's own ILRIA antigen. It is to be understood that anti-ILRIA antibodies according to the present disclosure encompass both anti-ILRIA autoantibodies and anti-ILRIA alloantibodies, which are produced by an individual's immune system to a foreign (non-self) ILRIA antigen. For example, anti-IL1RA alloantibodies may generated in response to IL1RA originating from the fetus or from any exposure to a non-self IL1RA antigen, such as during an organ transplant)

The term “antigen” refers to a molecule, such as a polypeptide, containing one or more epitopes (either linear, conformational or both) that can stimulate a subject's immune system to produce antigen-specific immune response. A polypeptide epitope may include between about 7 and 15 amino acids, such as, 9, 10, 12 or 15 amino acids. For example, the expression “IL1RA antigen” or “IL1RA antigenic polypeptide” may refer to a polypeptide of IL1RA (such polypeptides are discussed in more detail elsewhere in the present disclosure). The term “antigen” may be used interchangeably with the term “immunogen.” The expression “antigenic polypeptide,” which can be used interchangeably with the expression “immunogenic polypeptide,” is used in the present disclosure to refer to a polypeptide containing one or more epitopes that can stimulate a subject's immune system to produce antigen-specific immune response to the antigenic polypeptide. The term “epitope” or other related terms and expressions may be used in the present disclosure to polypeptides and/or amino acid sequences (which need not be contiguous) that are specifically recognized by antibodies. In other words, an antibody can specifically bind to an epitope, although an epitope, by itself, may not necessarily stimulate a subject's immune system to produce antigen-specific immune response. For example, the expression “IL1RA epitope” may refer to an IL1RA polypeptide or amino acid sequence that is recognized by anti-IL1RA antibodies, but such IL1RA epitope may or may not stimulate a subject's immune system to produce anti-IL1RA antibodies.

The terms “individual”, “subject”, “person,” and “patient” can be used interchangeably in the present disclosure to refer to a non-human animal or a human. Examples of subjects include, but are not limited to: humans and other primates, including non-human primates, such as chimpanzees and other apes and monkey species; farm animals, such as cattle, sheep, pigs, goats and horses; domestic mammals such as dogs and cats; laboratory animals including rodents, such as mice, rats and guinea pigs; birds, including domestic, wild and game birds, such as chickens, turkeys and other gallinaceous birds, ducks, geese, and the like. The terms individual, subject, and patient, by themselves, do not denote a particular age, sex, race, or clinical status. Thus, subjects of any age, whether male or female, are intended to be covered by the present disclosure. Likewise, the methods of the present invention can be applied to any human race, including, for example, Caucasian (white), African-American (black), Native American, Native Hawaiian, Hispanic, Latino, Asian, and European.

In some embodiments of the present disclosure, the subject may be a pregnant subject. In some embodiments, the subject is a primigravida pregnant subject. In some embodiments, the subject is a multigravida pregnant subject. In some embodiments, the subject is not pregnant. In some embodiments, the subject, who may or may not be pregnant, has previously been pregnant. In some embodiments, the subject, who may or may not be pregnant, has previously experienced or is experiencing pregnancy complications, such as, but not limited to, premature labor, premature delivery (including medically indicated premature delivery), premature rupture of membranes, and/or one or more miscarriages.

The term “premature” in the context of the present disclosure can be used interchangeably with the term “preterm” and indicates pregnancy complications, such as, but not limited to, labor, delivery, membrane rupture, pregnancy loss (miscarriage), that occur prior to 37 weeks of gestation.

The terms “administering” or “administration,” when using in the context of the present disclosure (and the related terms and expression), refer to the act of physically delivering a substance as it exists outside the body (for example, an immunogenic composition described in the present disclosure) into a subject. Administration can be by mucosal, intradermal, intravenous, intramuscular, subcutaneous delivery and/or by any other known methods of physical delivery. Administration encompasses direct administration, such as administration to a subject by a medical professional or self-administration, or indirect administration, which may be the act of prescribing a composition described in the present disclosure.

As used herein, the term “biological sample” encompasses any sample obtained from a biological source. A biological sample can, by way of non-limiting example, include blood, serum, plasma, tissue biopsy, amniotic fluid, cerebrospinal fluid, sera, urine, feces, epidermal sample, skin sample, cheek swab, amniotic fluid, cultured cells, bone marrow sample and/or chorionic villi. The term biological sample encompasses samples which have been processed to release or otherwise make available a protein for detection as described herein. A biological sample may be processed prior to use in a detection assay including dilution, addition of buffer or preservative, concentration, purification, or partial purification. Fixed or frozen tissues also may be used.

The term “placenta” and related terms refer to a temporary vascular organ in mammals, which connects the umbilical cord of the developing fetus to the wall of uterus, and mediates its metabolic exchange between the fetus and the maternal blood supply through association of placental tissues with uterine mucosa. The placenta's villous folds and branched microvilli offer an expansive surface area for active, bi-directional, maternal-fetal crosstalk. Placental antigens are transmitted directly into maternal blood stream, as observed by the translocation of exosomes, syncytial knots, and proteins, as well as small volumes of fetal blood in uncomplicated, healthy pregnancies. The placenta is also a site for antibody transport for passive immunization of the offspring. Maternal immunoglobulin G (IgG) is actively transcytosed across the syncytiotrophoblasts by binding to the neonatal Fc receptor within endosomes and secreted to fetal blood. Placenta not only sheds alloantigen that must be rapidly tolerated by the maternal immune system, it also actively and selectively recruits antibodies.

### IL1RA Polypeptides

Interleukin-1 receptor antagonist (IL1RA, gene name ILIRN) is a competitive inhibitor of the ILI receptor. IL1RA binds to ILIR1 with similar affinity as ILIA and IL1B but does not have agonist activity. IL1RA binding to ILIR1 does not trigger the association of IL-1 receptor accessory protein and the formation of a heterodimer. The entire IL1RA protein can used in embodiments according to the present disclosure, a fragment of the IL1RA protein may be used, a variant of IL1RA protein may be used, or a combination of two or more of the full-length polypeptide, a fragment, a variant of the full-length polypeptide, or a variant of a fragment of IL1RA protein may be used. In reference to the embodiments of the present invention, an amino acid sequence of IL1RA polypeptide may contain a naturally a naturally occurring (or “wild-type”) amino acid sequence of IL1RA protein or a portion (fragment) thereof. An example of a wild-type amino acid sequence of IL1RA protein is SEQ ID NO:1, the sequence represented by UniProt/UniProtKB Database Entry P18510. It is to be understood that, in some examples of IL1RA polypeptides according to the present disclosure, various features (such as posttranslational modifications) and mutations of the wild-type amino acid sequences of a IL1RA protein may be present in IL1RA polypeptide.

IL1RA protein amino acid sequence
SEQ ID NO: 1
MEICRGLRSHLITLLLFLFHSETICRPSGRKSSKMQAFRIWDVNQKTFYL
RNNQLVAGYLQGPNVNLEEKIDVVPIEPHALFLGIHGGKMCLSCVKSGDE
TRLQLEAVNITDLSENRKQDKRFAFIRSDSGPTTSFESAACPGWFLCTAM
EADQPVSLTNMPDEGVMVTKFYFQEDE

Some embodiments of IL1RA polypeptides may contain artificially modified amino acid sequences of IL1RA protein or portion (fragment) thereof. Artificially modified amino acid sequences of IL1RA polypeptides may contain various mutations and/or amino acid modifications, as compared wild-type sequences. IL1RA polypeptide amino acid sequences containing mutations with respect to wilt-type IL1RA polypeptide sequences can be referred to as “variants” and are discussed in more details below. In one example, an artificially modified amino acid sequence of an IL1RA polypeptide may contain mutations removing or adding glycosylation sites. In another example, an artificially modified amino acid sequence of an IL1RA polypeptide may contain one or more mutations stabilizing a desirable conformation of a an IL1RA polypeptide. An IL1RA polypeptide may be artificially chemically modified, for example, with glutaraldehyde. In some embodiments according to the present disclosure, an IL1RA polypeptide includes an epitope that is recognized by anti-IL1RA antibodies. It is to be understood that such IL1RA epitopes, while recognized by anti-IL1RA antibodies, may or may not stimulate a subject's immune system to produce anti-IL1RA antibodies.

The term “variant” of IL1RA polypeptide or protein refers to a polypeptide comprising an amino acid residue sequence that is at least 70, 75, 80, 85, 90, 92, 94, 95, 96, 97, 98 or 99% identical to a corresponding wild-type sequence of the IL1RA polypeptide. In certain embodiments according to the present disclosure, a variant of the IL1RA protein retains its ability to be bound specifically under appropriate conditions by an anti-IL1RA antibody that would specifically bind to the corresponding full length IL1RA polypeptide under appropriate conditions. In some instances, variants are modified at amino acid residues other than those essential for the biological activity, for example the ability of the polypeptide bind specifically to an IL1RA-specific antibody. In some instances, one or more such essential amino acid residues may optionally be replaced in a conservative manner or additional amino acid residues may be inserted such that the biological activity of the variant polypeptide is preserved.

The term “fragment” or “portion” with regard to an IL1RA polypeptide refers to an amino acid residue sequence of a portion of the full-length IL1RA protein, encompassing, for example, an amino acid residue sequence that is truncated at one or both termini by one or more amino acids. In certain embodiments, an IL1RA polypeptide fragment retains its ability to bind specifically under appropriate binding conditions by an anti-IL1RA antibody that would bind specifically to the corresponding full-length IL1RA protein under appropriate binding conditions. A portion of the IL1RA protein can be a polypeptide that is, for example, 10, 25, 50, 100, 150, 200, 250 or more amino acid residues in length of the full length IL1RA protein. Alternatively or in addition, such peptide sequence may comprise one or more internal deletions of one or more amino acid residues. Thereby the residual length of the fragment equals or exceeds the length of one or more continuous or conformational epitopes, e.g., 6, 7, 8, 9, 10, 11, 12, 13, 14, 15, 16, 17, 18, 19, 20, 21, 22, 23, 24, 25, 26, 27, 28, 29, 30, 21, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more amino acid residues.

In some embodiments, a fragment comprises at least 6 contiguous amino acid residues of SEQ ID NO:1 or an amino acid sequence having at least 90%, 91%, 92%, 93%, 94%, 95%, 96%, 97%, 98%, or 99% sequence similarity to SEQ ID NO:1. In some embodiments, a fragment comprises at least 8 contiguous amino acid residues of SEQ ID NO:1 or an amino acid sequence having at least 90%, 91%, 92%, 93%, 94%, 95%, 96%, 97%, 98%, or 99% sequence similarity to SEQ ID NO: 1. In some embodiments, a fragment comprises at least 12 contiguous amino acid residues of SEQ ID NO:1 or an amino acid sequence having at least 90%, 91%, 92%, 93%, 94%, 95%, 96%, 97%, 98%, or 99% sequence similarity to SEQ ID NO:1. In some embodiments, a fragment comprises 8-20 contiguous amino acid residues of SEQ ID NO: 1 or an amino acid sequence having at least 90%, 91%, 92%, 93%, 94%, 95%, 96%, 97%, 98%, or 99% sequence similarity to SEQ ID NO:1. In some embodiments, a fragment comprises 30-60 contiguous amino acid residues of SEQ ID NO:1 or an amino acid sequence having at least 90%, 91%, 92%, 93%, 94%, 95%, 96%, 97%, 98%, or 99% sequence similarity to SEQ ID NO:1. In some embodiments, a fragment comprises 16 contiguous amino acid residues of SEQ ID NO: 1 or an amino acid sequence having at least 90%, 91%, 92%, 93%, 94%, 95%, 96%, 97%, 98%, or 99% sequence similarity to SEQ ID NO:1. In some instances, a plurality of fragments is provided, each fragment comprising 49 contiguous amino acid residues of SEQ ID NO:1.

In some embodiments, a fragment comprises at least 6 contiguous amino acid residues of the sequence LQLEAVNITDLSENRK (SEQ ID NO:2) or an amino acid sequence having at least 90%, 91%, 92%, 93%, 94%, 95%, 96%, 97%, 98%, or 99% sequence similarity to SEQ ID NO:2. In some embodiments, a fragment comprises at least 7 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 8 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 9 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 10 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 11 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 12 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 13 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 14 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises at least 15 contiguous amino acid residues of SEQ ID NO:2 or an amino acid sequence having at least 90%, 91%, 92%, 93%, 94%, 95%, 96%, 97%, 98%, or 99% sequence similarity to SEQ ID NO:2. In some embodiments, a fragment comprises SEQ ID NO:2 or an amino acid sequence having at least 90%, 91%, 92%, 93%, 94%, 95%, 96%, 97%, 98%, or 99% sequence similarity to SEQ ID NO:2.

Variants and fragments according to the present disclosure may be prepared, for example, by introducing deletions, insertions or substitutions in nucleic acid sequences encoding them, or by chemical synthesis or modification. Moreover, variants and fragments may also be generated by fusion with other known polypeptides or variants thereof and encompass active portions or domains, preferably having a sequence identity of at least 70, 75, 80, 85, 90, 92, 94, 95, 96, 97, 98 or 99% when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, but retains at least some of the biological activity. For example, an active portion of a polypeptide retains the ability to bind to an anti-IL1RA antibody.

In some embodiments, the IL1RA polypeptide is an isolated, purified IL1RA polypeptide. Protein expression and purification methods are well known. IL1RA polypeptides according to the present disclosure may be provided in any form and at any degree of purification, from tissues or cells comprising said polypeptides in an endogenous form, such as cells overexpressing the polypeptide and crude or enriched lysates of such cells, to purified and/or isolated polypeptides that are essentially pure. In some embodiments, the IL1RA polypeptide is included in a phage display or eukaryotic cell display library. In some embodiments, the IL1RA polypeptide is heterologously-expressed on the surface of a cell. In some embodiments, IL1RA polypeptides have a native configuration, wherein the term “native configuration,” as used herein, refers to a folded polypeptide, such as a folded polypeptide purified from tissues or cells, such as mammalian cells or tissues or from non-recombinant tissues or cells. In some embodiments IL1RA polypeptides are recombinant proteins, wherein the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. Such techniques are well known in the art.

In some instances, IL1RA polypeptides can be denatured such as by heating, freezing or ultraviolet ray, or chemical treatments such as a surfactant or a denaturant. For example, such a denatured form of an IL1RA polypeptide may be prepared by treating it with sodium dodecyl sulfate (SDS) or dithiothreitol (DTT). IL1RA polypeptides that are included in a kit or a panel as described in the present disclosure can be provided within a cell, in a solution in which they are soluble, or the IL1RA polypeptides can be provided in a lyophilized form.

In some embodiments, IL1RA polypeptides can be immobilized on a solid carrier insoluble in an aqueous solution, such as via a covalent bond, electrostatic interactions, encapsulation or entrapment, for example by denaturing a globular polypeptide in a gel, or via hydrophobic interactions such as via one or more covalent bonds. Various suitable carriers, for example paper, metal, silicon or glass surfaces, microfluidic channels, membranes, beads such as magnetic beads, column chromatography media, biochips, polyacrylamide gels and the like have been described in the literature, for example in Kim et al. “Protein immobilization techniques for microfluidic assays.” Biomicrofluidics 7 (4): 041501 (2013). This way, the immobilized molecule, together with the insoluble carrier, may be separated from an aqueous solution in a straightforward manner, for example by filtration, centrifugation or decanting. An immobilized molecule may be immobilized in a reversible or irreversible manner. For example, the immobilization is reversible if the molecule interacts with the carrier via ionic interactions that can be masked by addition of a high concentration of salt or if the molecule is bound via a cleavable covalent bond such as a disulfide bridge which may be cleaved by addition of thiol-containing reagents. By contrast, the immobilization is irreversible if the molecule is tethered to the carrier via a covalent bond that cannot be cleaved in aqueous solution, for example a bond formed by reaction of an epoxide group and an amine group as frequently used to couple lysine side chains to affinity columns. An IL1RA polypeptide may be indirectly immobilized, for example by immobilizing an antibody or other entity having affinity to the molecule, followed by formation of a complex to the effect that the molecule-antibody complex is immobilized. Various reagents and kits for immobilization reactions are commercially available such as, for example, from Pierce Biotechnology.

### Methods of Detection

In various embodiments of the methods according to the present disclosure, one or more polypeptides identified by the inventors (some of which are listed in Table 2 of the present disclosure), including IL1RA polypeptides, can be used in various immunological techniques to detect antibodies associated with a risk of premature labor, including ILIR-specific antibodies. IL1RA protein and IL1RA polypeptides are discussed elsewhere in the present disclosure. The entire IL1RA protein can used in the provided methods, a fragment of the IL1RA protein may be used, a variant of IL1RA protein may be used, or a combination of two or more of the full-length polypeptide, a fragment, a variant of the full-length polypeptide, or a variant of a fragment of IL1RA protein may be used. For example, IL1RA polypeptides can be used in an immunoassay to detect anti-IL1RA antibodies in a biological sample. IL1RA polypeptides used in an immunoassay can be in a cell lysate (such as, for example, a whole cell lysate or a cell fraction), or purified IL1RA polypeptides can be used provided that such polypeptides contain at least one epitope recognized IL1R-specific autoantibodies (such as anti-IL1RA antibodies) remains available for binding.

In some embodiments according to the present disclosure, provided are methods of detecting the presence of an antibody associated with a risk of premature labor, such as an anti-IL1RA antibody, in a biological sample from a subject, comprising the steps of contacting the biological sample with a polypeptide identified by the inventors and described elsewhere in the present disclosure, such as an IL1RA polypeptide, and detecting the presence of binding of the polypeptide, such as the IL1RA polypeptide to antibodies, such as anti-IL IRA antibodies, in the biological sample. In some embodiments according to the present disclosure, provided are methods of identifying a subject at risk premature labor, comprising the steps of contacting the biological sample with an IL1RA polypeptide and detecting the presence of binding of the IL1RA polypeptide to anti-IL1RA antibodies in the biological sample.

In some embodiments of the present disclosure, a biological sample is assessed for the presence of antibodies associated with a risk of premature labor, such as anti-IL1RA antibodies, by contacting a biological sample obtained from a sample with a polypeptide identified by the inventors, such as an IL1RA polypeptide. In some embodiments, a liquid sample comprising antibodies associated with a risk of premature labor, such as anti-IL1RA antibodiesm from a subject may be used to practice the methods provided herein. Exemplary liquid samples include cell lysate, blood, serum, plasma, cerebrospinal fluid (CSF), amniotic fluid, or urine. A step of contacting a liquid sample comprising antibodies associated with a risk of premature labor, such as anti-IL1RA antibodies, with one or more polypeptides identified by the inventors, such as an IL1RA polypeptide, may be carried out by incubating an immobilized form of the polypeptide, such as an IL1RA polypeptide in the presence of the liquid sample under conditions that are compatible with the formation of an antibody-polypeptide complex, such as a complex comprising the IL1RA polypeptide and anti-IL1RA antibodies. Optionally, one or more washing steps may be contemplated.

In some embodiments, a biological sample is contacted with one or more polypeptides identified by the inventors, such as an IL1RA polypeptide, and a secondary antibody. The secondary antibody is an antibody raised against the IgG of the animal species in which the primary antibody originated. Secondary antibodies bind to the primary antibody to assist in detection, sorting and purification of target antigens to which a specific primary antibody is first bound. The secondary antibody must have specificity both for the antibody species as well as the isotype of the primary antibody being used. For example, if an anti-IL1RA antibody is present in the biological sample, under appropriate conditions, a complex is formed between the IL1RA polypeptide, the anti-IL1RA antibody in the biological sample, and the secondary antibody.

In some instances, the secondary antibody may be conjugated to a detectable label. Detectable labels are well known and include, without limitation, a fluorescent label, an enzymatic label, a radioactive label, a luminescent label, or an affinity tag such as biotin or streptavidin. Exemplary fluorescent dyes include water-soluble rhodamine dyes, fluoresceins, 2′,7′-dichlorofluoresceins, fluorescein isothiocyanate (FITC), DyLight™ 488 (Rockland Immunochemicals, Pottstown, Pensilvanya), phycoerythrin (PE), propidium iodide (PI), PerCP, PE-Alexa Fluor® 700 (Thermo Fisher Scientific Inc., Waltham, Massachusetts), Cy5, allophycocyanin, Cy7, benzoxanthene dyes, and energy transfer dyes, as disclosed in the following references: Handbook of Molecular Probes and Research Reagents, 8^{th} ed. (2002), Molecular Probes, Eugene, OR; U.S. Pat. Nos. 6,191,278, 6,372,907, 6,096,723, 5,945,526, 4,997,928, and 4,318,846; and Lee et al., 1997, Nucleic Acids Research 25:2816-2822. Exemplary enzymatic labels include but are not limited to alkaline phosphatase (AP) and horseradish peroxidase (HP)). Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Suitable radioactive labels include, for example, ³²P, ³³P, ¹⁴C, ¹²⁵I, ¹³¹I, ³⁵S, and ³H. In some instances, the detectable label can be a heterologous polypeptide such as an antigenic tag such as, for example, FLAG, polyhistidine, hemagglutinin (HA), glutathione-S-transferase (GST), or maltose-binding protein (MBP) for use in purifying IL1RA polypeptides. In some instances, the detectable label can be a heterologous polypeptide that is useful as diagnostic or detectable marker such as, for example, luciferase, a fluorescent protein (such as a green fluorescent protein (GFP)), or chloramphenicol acetyl transferase (CAT). Another labeling technique which may result in greater sensitivity is the coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

An antibody-polypeptide complex, such as complex comprising an anti-IL1RA antibody and IL1RA polypeptide, may be detected using a variety of methods, such as, but not limited to, immunofluorescence microscopy or spectroscopy, luminescence, nuclear magnetic resonance (NMR) spectroscopy, immunodiffusion, radioactivity, chemical crosslinking, surface plasmon resonance, native gel electrophoresis, or enzymatic activity. Depending on the nature of the sample, either or both immunoassays and immunocytochemical staining techniques may be used. Enzyme-linked immunosorbent assays (ELISA), Western blot, and radioimmunoassays can be used to detect antibodies associated with a risk of premature labor, such as anti-IL1RA antibodies, in a biological sample. While some of these methods allow for direct detection of the antibody-polypeptide complex, such as a complex of anti-IL1RA antibody and IL1RA polypeptide, in some embodiments, the secondary antibody is labeled such that the complex may be detected specifically owing to intrinsic properties of the label such as, for example, fluorescence, radioactivity, enzymatic activity, visibility in NMR or magnetic resonance imaging (MRI) spectra or the like. In some embodiments, the detection method may include, but is not limited to, performing any one or more of Western blot, dot blot, protein microarray, ELISA, line blot radioimmune assay, immunoprecipitation, indirect immunofluorescence microscopy, radioimmunoassay, radioimmunodiffusion, ouchterlony immunodiffusion, rocket immunoelectrophoresis, immunohistostaining, complement fixation assay, fluorescence-activated cell sorting (FACS), and protein chip. antibodies associated with a risk of premature labor, such as anti-IL1RA antibodies, in a biological sample may be detected by immunohistochemistry. Immunohistochemical methods are well known, and non-limiting exemplary methods are described in U.S. Pat. Nos. 5,073,504; 5,225,325; and 6,855,552. See also Dabbs, Diagnostic Immunohistochemistry, 2^{nd} Ed., 2006, Churchill Livingstone; and Chu & Weiss, Modern Immunohistochemistry, 2009, Cambridge University Press. It is to be understood that immunohistochemistry routinely includes steps that are not necessarily discussed herein in detail, such as washing the tissue samples to remove unbound secondary antibodies and the parallel staining experiments with proper controls.

In some embodiments, a method according to the present disclosure involves contacting one or more polypeptide identified by the inventors, such as an IL1RA polypeptide, with a biological sample from a subject and a secondary antibody having a suitable label under conditions in which an antibody-polypeptide complex is formed, such as a complex between the IL1RA polypeptide, a corresponding anti-IL1RA antibody in the biological sample, if present, and the secondary antibody; and detecting the complex formed, if formed, by detecting the label of the secondary antibody, wherein the presence of the secondary antibody is indicative of the presence of an antibody associated with a risk of premature labor, such as an anti-IL1RA antibody, in the biological sample, and wherein the absence of the secondary antibody is indicative of the absence of an antibody associated with a risk of premature labor, such as an anti-IL1RA antibody, in the biological sample. In some instances, the secondary antibody is detectably-labeled. Immobilization of the appropriate polypeptides, such as an IL1RA polypeptide, on a solid carrier can facilitate the method of antibody detection. In some instances, the method comprises contacting one or more polypeptides identified by the inventors, such as an IL1RA polypeptide, having a suitable label thereon with a biological sample from a subject, immunoprecipitating any complex formed between the polypeptide and the antibody, such as a complex of the IL1RA polypeptide and a corresponding anti-IL1RA antibody, in the biological sample, and monitoring for said label on any of said complexes, wherein the presence of said label is indicative of the presence of an antibody associated with a risk of premature labor, such as an anti-IL IRA antibody, in the biological sample and the absence of said label is indicative of the absence of an antibody associated with a risk of premature labor, such as an anti-IL1RA antibody, in the biological sample. In some instances, the method comprises a combination of immunoprecipitation and Western blot analysis to detect the presence of antibodies associated with a risk of premature labor, such as an anti-IL1RA antibody, in a biological sample from a subject. For example, the method may comprise contacting one or more polypeptides identified by the inventors, such as an IL1RA polypeptide or fragment or variant thereof, with a biological sample from a subject under conditions in which a complex is formed between polypeptide and the antibody, such as the IL1RA polypeptide and a corresponding anti-IL1RA antibody, in the biological sample, if present; immunoprecipitating any complex formed, such as the complex between the IL1RA polypeptide and a corresponding anti-IL1RA antibodym in the biological sample to produce an immunoprecipitate comprising any such complex formed; separating components of the immunoprecipitate from each other (e.g., by electrophoresis), said components comprising the polypeptide and the corresponding antibody, such as the IL1RA polypeptide and a corresponding anti-IL1RA antibody, in the biological sample, if present; and contacting the components of the immunoprecipitate with a secondary antibody having a suitable label thereon that specifically binds to a constant region of the antibody, such as the anti-IL1RA antibody, if present; and detecting the complex formed, if formed, by detecting the label of the secondary antibody, wherein the presence of the secondary antibody is indicative of the presence of an antibody associated with a risk of premature labor, such as an anti-IL1RA antibody, in the biological sample, and wherein the absence of the secondary antibody is indicative of the absence of an antibody associated with a risk of premature labor, such as an anti-IL IRA antibody in the biological sample. For example, immunoprecipitation assay may be performed to detect the presence of antibodies associated with a risk of premature labor, such as anti-IL1RA antibodies, in a subject by contacting a polypeptide identified by the inventors, such as a recombinant IL1RA polypeptide, with a biological sample from the subject, such as serum. Exemplary labels include any of the detectable labels described in this disclosure including, for example, but not limited to, fluorescent dyes and radioactive labels.

In some embodiments of the methods according to the present disclosure, a polypeptide, such an IL1RA polypeptide, is heterologously expressed. Heterologous expression is accomplished by introduction of a nucleic acid encoding for a protein of interest from one species into the cell of another species (“host cell”), such that the host cell's cellular machinery expresses the foreign protein. For example, a heterologously expressed IL1RA polypeptide is an IL1RA polypeptide expressed by a host cell, into which a nucleic acid encoding for the IL1RA polypeptide from a species different from the host cell species was introduced by appropriate molecular biology techniques.

In some embodiments of the methods according to the present disclosure, a polypeptide, such as an IL1RA polypeptide is heterologously expressed on the surface of a cell. For example, a vector comprising the coding sequence of a polypeptide, such as an IL1RA polypeptide, operably linked to a promoter can be introduced into a cell. The vector may comprise elements that cause the polypeptide, such as the IL1RA polypeptide, to be expressed on the surface of the cell. For example, the polypeptide, such as the IL1RA polypeptide, may be expressed as fusion proteins with a membrane protein on the surface of the cell. In some instances, the cell is a bacteria cell or a eukaryotic cell. For example, the eukaryotic cell may be a yeast cell or a mammalian cell such as a human cell. Methods of transfection and transduction of cells to introduce recombinant nucleic acids are well known. For example, a 293T cell-based expression assay can be used to detect antibodies associated with a risk of premature labor, such as anti-IL1RA antibodies, in a biological sample from a subject, such as serum.

In instances where the polypeptide used in the methods according to the present disclosure, such as an IL1RA polypeptide, is in a phage display or eukaryotic cell display library, the presence of an antibody associated with a risk of premature labor, such as an anti-IL1RA antibody, in a biological sample from a subject is assessed by contacting the biological sample with a phage display or eukaryotic cell display library. An appropriate display library includes a plurality of eukaryotic cells or phage that express a plurality of peptides, including various polypeptides identified by the inventors, such as IL1RA polypeptides, on the surface of the eukaryotic cells or phage. For example, various IL1RA polypeptides may be expressed as fusion proteins with a membrane protein on the surface of the eukaryotic cells or phage. Each cell or phage in the library expresses a different IL1RA polypeptide. In some instances, the eukaryotic cell may be a yeast cell or a mammalian cell such as a human cell. The biological sample can be assayed to detect whether there is specific protein-protein interaction with any of the peptides expressed on the surface of the eukaryotic cells or phage. Methods of detecting protein-protein interactions using phage display are well-known in the art. For example, a polypeptide comprising a putative epitope may be bound to a solid support and the phage library applied thereto. After washing the solid support, any phage that remain bound to the solid support may express an anti-IL1RA antibody that can bind specifically to an IL1RA polypeptide. The phage DNA is isolated (after bacterial amplification) and sequenced to identify the sequence of the peptide expressed by the phage. Such peptides may then be further assessed individually for specific binding to the putative immunogen such as, for example, by immunoprecipitation, Western blot, or other immunoassay. In some instances, where the display library comprises eukaryotic cells, specific protein-protein interaction with any of the peptides may be assessed by flow cytometry. In some instances, the eukaryotic cells of the display library may be yeast cells. In some instances, the eukaryotic cells of the binding pool may be mammalian cells such as human cells. The peptides expressed on the cells of the display library may be fluorescently labeled (see discussion above regarding detectably labeled secondary antibodies for exemplary fluorescent labels). The biological sample and the display library may be combined, and FACS analysis performed to identify cells that express peptides that are bound specifically to antibody associated with a risk of premature labor, such as an anti-IL1RA antibody. In some instances, the identified cells may then be expanded in vitro, and the DNA or the RNA analyzed, such as by next generation sequencing. In some instances, single cell PCR may be performed followed by RNA and/or DNA sequence analysis. Other exemplary methods for assessing protein-protein interactions between a biological sample that contains an antibody associated with a risk of premature labor, such as an anti-IL1RA antibody, and a display library include those described in Jardine et al. “Rational HIV immunogen design to target specific germline B cell receptors.” Science 340 (6133): 711-716 (2013) and McGuire et al. “Diverse Recombinant HIV-1 Envs Fail to Activate B Cells Expressing the Germline B Cell Receptors of the Broadly Neutralizing Anti-HIV-1 Antibodies PG9 and 447-52D.” J. of Virology 88 (5): 2645-2657 (2014). See also WO 2020/190700, which is incorporated by reference for the teaching of the phage display system.

In some instances, more than one of the detection methods described above may be used in a complementary manner for more reliable results. In some embodiments, other immunoassays can be performed either in alternative to or before and/or after the immunohistochemistry methods. For example, a Western blot may be performed using, for example, a panel of known antigens associated with antibodies, the panel including an IL1RA polypeptide, the results of which may warrant further evaluation using, for example, the immunohistochemistry methods described herein. In another example, an immunohistochemistry method as described herein may be performed, followed by a Western blot in order to, for example, further confirm the specific antigens, including one or more polypeptides identified by the inventors, such as the IL1RA polypeptide, recognized by the antibodies in the biological sample. In another example, a phage or eukaryotic cell display library that includes a plurality of eukaryotic cells or phage that express a plurality of peptides, including one or more polypeptides identified by the inventors, such as IL1RA polypeptides on the surface of the eukaryotic cells or phage may be used to assess for the presence of antibodies associated with a risk of premature labor, such as anti-IL1RA antibodies, in the biological sample from the subject, and then followed by a radioligand binding assay method or an immunohistochemistry method as described herein. In another example, the biological sample may be assessed by a radioligand binding assay method first, with confirmation by assessing the sample using a phage or eukaryotic cell display library.

Any data demonstrating the presence or absence of an antibody associated with a risk of premature labor, such as an anti-IL1RA antibody, may be correlated with reference data. For example, detection of an anti-IL1RA antibody indicates that the subject who provided the sample is at risk of premature labor. If the subject has been previously identified as being at risk of premature labor, the amount of anti-IL1RA antibodies detected at the time of prior diagnosis and in the present time may be correlated to find out about the progression of the risk and/or the success of a treatment. For example, if the amount of anti-IL1RA antibodies is found to increase, it may be concluded that the risk of premature labor is increasing and/or that any treatment attempted is unsuccessful.

Kits, systems and devices useful for performing the methods described in this section are described elsewhere in the present disclosure.

### Kits, Devices, and Systems

Provided in this disclosure are kits and systems containing one or more polypeptides identified by the inventors (some of which are listed in Table 2 of the present disclosure), such as one or more IL1RA polypeptides, to which antibodies associated with a risk of premature labor, such as anti-IL1RA antibodies, can specifically bind. The polypeptides used in the kits according to the present disclosure binds to antibody associated with a risk of premature labor, such as anti-IL1RA antibodies, from subjects. A kit or a system according to embodiments of the present invention includes one or moreone or more polypeptides identified by the inventors, such as IL1RA polypeptides, according to the present disclosure. A kit or a system may also contain various other reagents for detecting antibodies associated with a risk of premature labor, such as anti-IL1RA antibodies, in a sample, such as a biological sample obtained from a subject. In some examples, a kit or a system may include one or more solubilizing agents for increasing the solubility of a polypeptide such as, for example, a buffer solution. The kit or the system may further include reagents provide a detectable signal when used in conjunction with one or more polypeptides identified by the inventors, such the IL1RA polypeptides, and a biological sample. In some embodiments, the kit or the system includes a detectably-labeled secondary antibody that is able to bind to an antibody associated with a risk of premature labor, such as an anti-IL1RA antibody, specifically binding to one or more polypeptides included in the kit, such as IL1RA polypeptides. Reagents for the detection of the secondary antibody label can also be included in the kit. The secondary antibody is detected by a method that depends on a labeling group used. Exemplary labels for secondary antibodies are described elsewhere in this disclosure. A kit or a system can include a device according to the present disclosure, as discussed further below.

In addition, a kit or a system can include directions for using the polypeptides included in the kit, such as IL1RA polypeptides, and/or directions for practicing a method described in the present disclosure, such as detecting antibodies associated with a risk of premature labor, for example, anti-IL1RA antibodies, in a biological sample. The concentration or amount of antibodies associated with a risk of premature labor, such as anti-IL1RA, antibodies contained in the biological sample is indirectly measured by measuring the amount of the detectable label. The obtained measurement value may be converted to a relative or absolute concentration, amount, activity, etc. using a calibration curve or the like.

In some embodiments, a kit or a system includes a reference sample, such as a normal control sample. In some embodiments, a kit or a system includes one or more control antibody that detects an antigen that is expected to be present in a biological sample such as, for example, a biological sample from a healthy subject.

In addition to the one or more polypeptides according to the present disclosure, such as IL1RA polypeptides, the kit or the system can include additional polypeptides such as, for example, positive or negative controls or other antigens known to bind to antibodies of prognostic and/or diagnostic value.

Some embodiments of the present invention include a prognostic or diagnostic device comprising one or more one or more polypeptides identified by the inventors, such as IL1RA polypeptides. In some embodiments, such a prognostic or diagnostic device comprises one or more polypeptides according to the present disclosure, such as IL1RA polypeptides, and allows contacting the polypeptides with an aqueous solution, such as liquid sample, in a straightforward manner. In particular, the one or more polypeptides according to the present disclosure, such as IL1RA polypeptides, may be immobilized on the surface of a carrier, which carrier comprises, but is not limited to glass plates or slides, chips, microtiter plates, fibers, beads, for example magnetic beads, chromatography columns, membranes or the like. Exemplary devices include line blots, microtiter plates and biochips. In some embodiments, the device can include additional polypeptides such as, for example, positive or negative controls or other antigens known to bind to antibodies of prognostic and/or diagnostic value.

### Methods of Treatment

Provided in the present disclosure are methods of treating subjects identified as at risk for premature labor using methods of detection described in the present disclosure, which are useful for screening subjects to identify those at risk of premature labor. The presence (or absence) of antibody associated with a risk of premature labor, such as an anti-IL1RA antibody, in a biological sample from a subject may be detected by using any of the methods, kits, or devices described in elsewhere in the present disclosure. In such cases, the provided methods of detection may be useful as an indicator in determining if a suitable treatment is appropriate for the subject at risk of premature labor. Detection of one or more antibodies associated with a risk of premature labor, such as the anti-IL1RA antibody, in a biological sample from the subject indicates that the subject may be at risk for premature labor. Accordingly, also provided methods of treating a subject having or harboring one or more antibodies associated with a risk of premature labor, such as IL1RA antibodies.

In some embodiments, a medical examination and/or testing of a subject thus identified as being at risk for premature labor may be performed to evaluate the presence of other clinical signs and/or symptoms of premature labor or those indicating that the subject is at risk of premature labor. Some of the signs and/or symptoms of premature labor in a subject may be tightening of the uterus (contractions), menstrual-type cramps, the feeling o pressure in the lower belly, backache, diarrhea, change in the type or amount of vaginal discharge, which can be blood, mucus, or watery fluid, leaking fluid from the vagina. Some of the signs and/or symptoms indicating that the subject is at risk of premature labor may be a presence of an infection, vaginal bleeding, stretching of a uterus, for example, due to having a large fetus or a large amount of amniotic fluid, history of smoking and/or using illegal drugs, age of subject (for example, being under 20 years or over 35 years old), long-term illness, for example, heart disease or kidney disease, abnormally shaped uterus, having a preterm birth in the past, belonging to a particular race or ethnic group (for example, being African American in the U.S.A.), having placenta separation from uterus, having abnormally shaped placenta, or premature rupture of membranes.

In some embodiments, various monitoring or testing procedures may be performed on a subject identified as being at risk for premature labor duet to the presence of one or more antibodies associated with a risk of premature labor, such as anti-IL1RA antibodies. Some non-limiting examples of such monitoring or testing procedures may be one or more of: monitoring contractions in the subject, for example, by using an electronic monitor; monitoring fetal heart rate in a subject; performing one or more cervical exams on the subject, performing transvaginal ultrasound exam (which may be used to measure the length of the cervix in the subject); testing cervical or vaginal sample for amniotic fluid to determine if the amniotic sac is ruptured; testing cervical or vaginal fluid for fetal fibronectin (fFN) (detection of fFN in cervical or vaginal fluid may be a sign of premature labor).

In some embodiments, the methods according to the present disclosure may include performing one or more treatments on a subject having or harboring one or more antibodies associated with a risk of premature labor, such as IL1RA antibodies, and thus identified as being at risk of premature labor. In a pregnant subject, such treatments may be intended to prevent premature labor or to promote fetal maturation. For example, the subject may be put on bedrest. The subject may be administered one or more tocolytic medications (which can also be called “also called anti-contraction medications” or “labor suppressants” and can assist in delaying preterm delivery by suppressing uterine muscle contractions), some non-limiting examples of which are include β2 agonists, calcium channel blockers, non-steroidal anti-inflammatory drugs (NSAIDs), and magnesium sulfate. In one more example, a subject may be administered corticosteroids to help fetal lungs mature. In yet another example, cervical cerclage (stitching the cervix closed) may be performed on the subject. In one more example, antibiotics may be administered to a subject in an event an infection has been detected in the subject. It is to be understood that the above examples of various treatments are non-limiting, and that any combination of the treatments may be performed.

In some embodiments of the methods of treatment according to the present disclosure, a subject may be administered an immunosuppressive therapy. Immunosuppressive therapies are therapies that lower the activity of a subject's immune system. The immunosuppressive therapy administered to the subject may include at least one of the following: administration of an immunosuppressant drug, intravenous immunoglobulin administration, plasma exchange plasmapheresis, or immunoadsorption. For example, first-line immunosuppressive therapy can include corticosteroids, intravenous immunoglobulin, plasma exchange plasmapheresis, and immunoadsorption.

Plasma exchange plasmapheresis (also referred to as PLEX) may also be used. PLEX removes antibodies and other pathologic substances in the plasma. PLEX also alters the immune system by changing lymphocyte numbers and their distribution, T-suppressor cell function, and T-helper cell phenotypes. Steroids alone can be insufficient to ameliorate antibody-mediated immune process, and direct removal or neutralization of antibodies from the circulation by PLEX and IVIg may show a synergistic effect. In addition, PLEX increases the proliferation of antibody-producing cells, and this could increase susceptibility of these cells to immunosuppressants and chemotherapeutic agents. Immunoadsorption is a refined form of PLEX that enables the selective removal of immunoglobulins rom separated plasma through high-affinity adsorbers (e.g., Protein A).

In some instances, treatment of the subject includes reducing the amount of antibodies associated with a risk of premature labor, such as anti-IL1RA antibodies, in the subject. For example, the amount of antibodies associated with a risk of premature labor, such as anti-IL1RA antibodies, may be reduced by plasmapheresis, which involves the removal of blood from the subject, separation of the plasma from blood cells, and reinjection of the cells. Plasmapheresis may be carried out by plasma exchange, in which the plasma removed is replaced in whole or in part by a plasma substitute such as lactated Ringer's solution, or by plasma perfusion, in which the antibodies are separated from the plasma and the plasma then returned to the subject.

Antibodies may be removed from the subject's plasma by any suitable technique, such as by contacting the plasma to a solid support having an immunoglobulin binding protein (e.g., protein A) immobilized thereon), removing the plasma from the solid support, and then returning the plasma to the subject. Typically, this method is practiced by immobilizing the immunoglobulin binding protein on an affinity support in an affinity column, passing the blood plasma through the affinity column, and returning the plasma to the subject (optionally, but preferably, recombining the plasma with the patient's blood cells). The method may also be carried out on whole blood or other suitable blood fraction, which may then be recombined with blood cells and then returned to the patient, as will be appreciated by persons skilled in the art.

In a variation of the foregoing, one or more polypeptides according to the present disclosure, such as IL1RA polypeptides may be immobilized on the solid support, and the antibodies associated with a risk of premature labor, such as anti-IL1RA antibodies, selectively removed from the subject's blood by contacting the subject's blood, blood plasma, or other suitable fraction to the solid support, as described above. Such procedures advantageously avoid substantial reduction in levels of other antibodies in the subject undergoing treatment.

In some instances, treatment of the subject harboring anti-IL1RA antibodies in the subject may involve administering IL1RA or its analogues, which may mitigate the effects of anti-IL IRA antibodies on IL1RA signaling. In some embodiments, treatment of the subject harboring anti-IL1RA antibodies in the subject may involve administering IL1RA antagonists, such as, but limited to, anakinra.

In accordance with the present disclosure, there may be employed conventional molecular biology, microbiology, biochemical, and recombinant DNA techniques within the skill of the art. Such techniques are explained fully in the literature. The provided methods and compositions will be further described in the following examples, which do not limit the scope of the methods and compositions of matter described in the claims.

Methods of Identifying Autoantibodies Associated with a Risk of Premature Labor

Methods of identifying autoantibodies associated with a risk of premature labor are provided in the present disclosure. Embodiments of such methods may use PhIP-seq as described, for example, in are described, for example, in WO2020190700, WO2021138273, WO2021046466, and WO2022146837. Such methods may also be referred to as methods of identifying and/or detecting antigens of autoantibodies associated with a risk of premature labor. Embodiments of the methods may involve the following steps: (a) contacting a first biological sample obtained from a human subject who has undergone an uncomplicated term pregnancy with a peptide display library under conditions sufficient to permit binding of a first plurality of antibodies from the first biological sample to a first plurality of corresponding peptides within the peptide display library to generate a first plurality of antibody-peptide complexes; (b) contacting a second biological sample obtained from a human subject who has undergone a preterm pregnancy with the peptide display library under conditions sufficient to permit binding of a second plurality of antibodies from the first biological sample to a second plurality of corresponding peptides within the peptide display library to generate a second plurality of antibody-peptide complexes; (c) identifying the first plurality of the peptides and the second plurality of the peptides; and, (d) using a computer system, identifying the third plurality of peptides as the peptides found in the second plurality of the peptides but not in the first plurality of peptides. The third plurality of the peptides are the antigens of the autoantibodies associated with the risk of premature labor.

In some embodiments of identifying autoantibodies associated with a risk of premature labor, the peptide display library is a phage display library, and a nucleic sequence encoding each peptide of the phage display library includes a unique nucleic acid barcode sequence. The first plurality of complexes and the second plurality of complexes are subjected to nucleic acid amplification under conditions sufficient to amplify the unique nucleic acid barcode sequences, and the of the amplified nucleic acid sequences are then determined by a suitable sequencing method. The term “sequencing,” as used herein, and the related terms and expressions, generally refer to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads. A read may include a string of nucleic acid bases corresponding to a sequence of a nucleic acid molecule that has been sequenced. The determined sequences are then for the identifying the first plurality of the peptides and the second plurality of the peptides. As discussed above, the peptides found in the second plurality of the peptides but not in the first plurality of peptides are the antigens of the autoantibodies associated with the risk of premature labor. Some of these antigens are listed in Table 2 of the present disclosure.

### Computer Systems

Any of the computer systems mentioned in the present disclosure may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. The subsystems can be interconnected via a system bus. Additional subsystems such as a printer, keyboard, storage device(s), monitor, which is coupled to display adapter, and others are shown. Peripherals and input/output (I/O) devices, which couple to I/O controller, can be connected to the computer system by any number of means known in the art, such as serial port. For example, serial port or external interface (e.g. Ethernet, Wi-Fi, etc.) can be used to connect computer system to a wide area network such as the Internet, a mouse input device, or a scanner. The interconnection via system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware (e.g. an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### EXAMPLES

The following examples are offered to illustrate, but not to limit the claimed invention. Examples 2-5 were included in U.S. Provisional Application No. 63/504,626, filed May 26, 2023, and U.S. Provisional Application No. 63/564,699, filed Mar. 13, 2024, and describe the initial investigation of autoantibody markers of preterm birth. Examples 6-12 describe the updated investigation of autoantibody markers of preterm birth. Example 1 describes the methods used both in the initial and the updated investigations.

### Example 1: Methods

### A. Human Cohorts and Samples-Initial Investigation

Sera from the CA1000 cohort was obtained from a California-wide biobank (Baer et al. “Risk of early birth by body mass index in a propensity score-matched sample: A retrospective cohort study.” BJOG. 129 (10): 1704-1711 (2022)). Sera from the PPROM cohort was obtained from deliveries at UCSF under IRB approved protocols. A healthy, uncomplicated, term pregnancy was defined as one that ended greater than or equal to 37 weeks of gestation and did not have a diagnosis of any of the following: placental abruption, placenta previa, chorioamnionitis, oligohydraminos, polyhydraminos, rupture of membranes, bacterial vaginosis, urinary tract infection, diabetes or gestational diabetes, preexisting or gestational hypertension, preeclampsia, infection during pregnancy, any specified placental condition, retained placenta with or without hemorrhage, unspecified hemorrhage, abnormal clotting, or threatened abortion. Any pregnancy that required transfusion, rhesus isoimmunization, or cerclage was excluded from this group. Also excluded were mothers with any hypertension disorder, endometriosis, Group B streptococcus positivity, any coagulation deficiency, malignancy, asthma, allergic dermatitis, anaphylaxis, rheumatoid arthritis, systemic lupus erythromatosus, autoimmune thyroiditis. Mothers who reported any smoking, drug or alcohol abuse or dependency, or mental disorder during pregnancy were excluded. Pregnancies where the infant exhibited convulsions, abnormal neural imaging or exam, retinopathy of prematurity, respiratory distress syndrome, intraventricular hemorrhage, necrotizing enterocolitis, bronchopulmonary dysplasia, periventricular leukomalacia, hypoxic ischemic encephalopathy, or patent ductus arteriosus were also excluded from this group. It was required that healthy uncomplicated pregnancies did not have a history of recurrent pregnancy loss, preterm delivery, or poor obstetric/reproductive outcomes. Term pregnancies with one or more of the above were considered medically complicated. Preterm pregnancies were defined as those delivering prior to 37 weeks of gestation and were further subdivided into spontaneous, preterm premature rupture of membranes, or medically indicated preterm delivery. Healthy, non-pregnant controls were obtained from the New York Blood Bank (NYBB).

### B. Human Cohorts and Samples—Updated Investigation

Sera from Cohort I was obtained from a California-wide biobank (Baer et al. (2022)). Sera from Cohort II was obtained from deliveries at the University of California, San Francisco (UCSF) Medical Center (UCSF) under the protocols approved by the institutional review board (IRB). Sera from Cohort III was obtained from deliveries in San Francisco Bay Area hospitals under IRB approved protocols. Sera from Cohort IV was obtained from University College London (UCL) Hospital and Homerton University Hospital of the National Health Services of the United Kingdom, under the corresponding IRB-approved protocols. An uncomplicated, term pregnancy was defined as one resulting in delivery at greater than or equal to 37 weeks of gestation and not having a diagnosis of any of the following conditions: placental abruption, placenta previa, chorioamnionitis, oligohydraminos, polyhydraminos, rupture of membranes, bacterial vaginosis, urinary tract infection, diabetes or gestational diabetes, preexisting or gestational hypertension, preeclampsia, infection during pregnancy, any specified placental condition, retained placenta with or without hemorrhage, unspecified hemorrhage, abnormal clotting, or threatened abortion. Any pregnancy that required transfusion, rhesus isoimmunization, or cerclage was excluded from this group. Also excluded were the mothers with any hypertension disorder, endometriosis, Group B streptococcus positivity, any coagulation deficiency, malignancy, asthma, allergic dermatitis, anaphylaxis, rheumatoid arthritis, systemic lupus erythematosus, autoimmune thyroiditis. Mothers who reported any smoking, drug or alcohol abuse or dependency, or mental disorder during pregnancy were also excluded. Pregnancies where the infant exhibited convulsions, abnormal neural imaging or exam, retinopathy of prematurity, respiratory distress syndrome, intraventricular hemorrhage, necrotizing enterocolitis, bronchopulmonary dysplasia, periventricular leukomalacia, hypoxic-ischemic encephalopathy, or known congenital heart disease were also excluded from this group. Finally, it was required that women defined as having uncomplicated pregnancies did not have a history of recurrent pregnancy loss, preterm delivery, or poor obstetric/reproductive outcomes. Term pregnancies with one or more of the above were considered medically complicated. Preterm pregnancies were defined as those delivering prior to 37 weeks of gestation and were further subdivided into spontaneous, preterm premature rupture of membranes, or medically indicated preterm delivery. Healthy, non-pregnant controls were obtained from the New York Blood Bank.

C. Human Peptidome Bacteriophage Immunoprecipitation Sequencing (PhIP-Seq) with Human Peptidome Library

The human T7 phage display library used for immunoprecipitation and sequencing is previously described and sera from the above cohorts was used in high-throughput protocols as previously described. Detailed protocols are described, for example, in WO2020190700, WO2021138273, and WO2022146837.

### D. Trapped Ion Mobility Time of Flight Mass Spectrometry of Immunoprecipitated Placental Protein Lysate (IP-MS)

Placenta was harvested within 2 hours of delivery and 1 cm³ blocks of tissue were flash frozen in liquid nitrogen. Placenta protein lysate was prepared by mechanically dissociating tissue using a glass Dounce homogenizer and an electric tissue homogenizer in RIPA buffer with protease inhibitors (Roche) on ice. Protein was quantified using Bradford Assay (Pierce), normalized to 500 μg in TNP40 and incubated with 1 μL of human sera overnight at 4 °C with overhead mixing. Proteins were subsequently incubated with a 1:1 mix of protein A and protein G Dynabeads (Thermo Fisher Scientific) for 1 hour and washed five times with RIPA and once with TrisHCI. A NanoDrop spectrophotometer (Thermo Fisher Scientific) was used to quantify protein. Supernatant was removed and proteins were then resuspended in 8M urea, 50 mM Tris (pH 8) before being subject to a standard on-bead protein digestion. The protein digestion procedure included disulfide reduction with 5 mM DTT, alkylation with 14 mM iodoacetamide, and an overnight digestion with Lys/C at a protein to protease ratio of 1:50. In order to obtain an optimal tryptic digestion, the urea concentration was then diluted to 2 M with 50 mM Tris (pH 8) followed by the addition of trypsin at a protein to protease ratio of 1:50, and a 4 hour incubation at room temperature. The peptide supernatant pH was then decreased to 2 with 7 μL formic acid (FA) and separated from the beads with a magnet. An extraction was performed on the beads by resuspension with 0.1% FA, the supernatant of which was added to the final sample. Peptide desalting was performed with the AssayMAP Bravo protein sample prep platform (Agilent Technologies) using a standard RPS peptide cleanup cartridges and protocol (Zhang et al. “Automated Ligand Purification Platform Accelerates Immunopeptidome Analysis by Mass Spectrometry.” J. Proteome Res. 20:393-408 (2021)). Approximately 25 ng of desalted peptides were analyzed on a timsSTOF SCP mass spectrometer (Bruker Corporation) coupled with an EASY-nLC 1200 LC system (Thermo Fisher Scientific). Peptides were separated by reverse-phase chromatography on a 25 cm column (75-μm inner diameter, packed with 1.6 μm C18 resin, AUR2-25075C18A-CSI; IonOpticks). Peptides were introduced into the mass spectrometer using a gradient starting with 2% to 8% buffer B (0.1% (v/v) formic acid in 80% acetonitrile) for 1 min followed by an increase to 25% buffer B for 25 min then an increase to 40% buffer B for 5 min at a flow rate of 100 nL/min and were ionized by CSI (captive spray ionization).

The samples were first analyzed by Parallel Accumulation-Serial Fragmentation Data-Dependent Acquisition (PASEF-DDA) with a duty cycle of 1.03 sec comprising one MS1 survey for every 5 PASEF MS2 ramps and a mass range between 100 and 1700 m/z. The Trapped Ion Mobility Spectrometry (TIMS) device ramp/accumulation time was set to 166 ms with a 100% duty cycle and a mobility range of 0.7 to 1.3 1/K0. Precursor ions from the survey scan were selected using an adjusted isolation width of 2 m/z below 700 m/z and 3 m/z above 800 m/z precursor mass-to-charge value in the quadrupole that aligns with the anticipated TIMS elution time relative to the ion mobility value. Fragmentation was performed by Collision Induced Dissociation (CID) with CE (collisional energy) interpolation between 42 eV at 0.65 1/K0, 31.92 eV at 0.8 1/K0, 36.96 eV at 1 1/K0, 42 eV at 1.2 1/K0, 47.04 eV at 1.4 1/K0 and 51.24 eV at 1.6 1/K0. Active exclusion of precursors were released after 0.2 minutes. Precursor repetitions were set to a target intensity of 20000 and a threshold of 500.

Real-time searching was performed with PaSER (Parallel Search Engine in Real-Time) against a human Uniprot database (downloaded on 30 Jul. 2022) using a reverse-decoy method with default settings. A spectral library was generated from the searched DDA data using PaSER. Samples were then analyzed by PASEF-Data Independent Acquisition (PASEF-DIA) using a high-speed acquisition scheme described in Meier et al. “diaPASEF: parallel accumulation-serial fragmentation combined with data-independent acquisition.” Nat Methods 17:1229-1236 (2020), and searched in real-time against the DDA generated spectral library using the TIMS DIA-NN algorithm with default settings.

Peptide abundances were collapsed to the corresponding protein, log-normalized, and fold changes and z-scores were calculated over mean signal in term pregnancies. Preterm-specific hits were identified by requiring that at least one preterm pregnancy and no term pregnancies met the threshold of z≥3 for a given protein.

E. ELISA with HEK293T IL1RA Overexpression Supernatant

HEK293T cells (ATCC-CRL3216) were plated at 30% density in complete DMEM media (Sigma) with glutamine, 10% Fetal Bovine Serum (FBS), 50 μg/mL of streptomycin, and 50 IU/ml of penicillin. The cells were plated on standard 10 cm² tissue culture dishes. Following plating, the cells were allowed to seed for 24 hours and subsequently transfected with experimental plasmids using a standard calcium phosphate transfection method. One microgram of pCMV6 IL1RA overexpression plasmid (Origene Cat. No. RC218518) was transfected per 10 cm² dish. 96 hours following transfection, the supernatant was harvested and normalized using a standard Bradford assay. Western blot analysis confirmed IL1RA expression in supernatant (data not shown). A sandwich ELISA was performed using standard protocols by capturing IL1RA from supernatant using goat anti-human IL1RA antibody (R&D Systems AF-280-NA) and detecting by utilizing a dilution of patient serum. Bound anti-IL IRA from human serum was detected with an anti-human HRP and quantified with standard protocols.

### F. Placental Immunofluorescence

Placental tissue was harvested within two hours of delivery, embedded, unfixed, in Tissue-Tek® O.C.T. compound (Sakura Finetek USA, Inc., Torrance, California), and flash-frozen in an isopentanol bath submerged in liquid nitrogen. Ten micron cryosections were cut from frozen blocks and adhered to glass slides. The slides were thawed and briefly post-fixed with acetone, rehydrated in 1×PBS, blocked with 10% goat serum in 1×PBS for 2 hours, and incubated with 1:1000 rabbit anti-human anti-IL1RA antibody (Millipore Sigma Cat No. HPA001482) and 1:500 rat anti-human CD31 antibody (Abcam Cat. No. ab9498) overnight at 4 °C in a humidified chamber. Primary antibodies were washed using standard protocols and incubated with anti-rabbit and anti-rat secondary antibodies using standard protocols. Secondary antibody-only controls were stained identically but omitting primary antibody. Stained tissue was imaged using the Crest LFOV Spinning Disk/C2 Confocal imaging system (Custom research instrument with Nikon and Crest Optics parts) at 400× magnification under identical camera exposure and laser settings for secondary antibody-only and experimental samples. Micrograph exposure was normalized to secondary antibody-only negative control in FIJI imaging packages (Schindelin et al. “Fiji: an open-source platform for biological-image analysis.” Nat Methods 9, 676-682 (2012) and applied to all images at once. In peptide blocking experiments, PrEST Antigen™ (Atlas Antibodies, Bromma, Sweden) used. Anti-IL1RA antibody was incubated with the antibody per manufacturer's instructions at 4 °C overnight and used to stain the tissue as described above.

### G. In Vitro IL1RA Blocking Assay

Diluted patient sera (1:20) was pre-incubated with human IL1RA (Peprotech) overnight, applied to the HEK-Blue IL1B cells (InvivoGen) for 2 hours, and stimulated with human IL1B (Peprotech®, Cranbury, New Jersey) for 72 hours at 37 °C. Supernatants were assayed using the QANTI-Blue™ assay (InvivoGen, San Diego, California).

### H. Murine IL1RA Dot Blot

500 or 250 ng of recombinant mouse IL1RA (R&D Systems 480-RM-50) were blotted onto nitrocellulose using a custom 3D printed dot blot device under vacuum. Membranes were blocked for 1 hour at room temperature in 5% (w/v) milk and subsequently probed overnight at 4 °C with anti-human IL1-RA antibodies (R&D Systems AF-280-NA and Sigma Prestige HPA001482), anti-mouse IL1RA antibodies (Invitrogen PA5-21776 or R&D Systems MAB4801), or no primary antibody. Following standard washing procedures, the membranes were probed with relevant secondaries conjugated to IRDye® infrared dyes (LI-COR Biosciences, Lincoln, Nebraska), and the blots were imaged using the Odyssey® system (LI-COR Biosciences, Lincoln, Nebraska). The membranes were imaged together in one scan under identical exposure settings.

### I. Animal Husbandry and Injections

All mice were housed, bred, and maintained in a pathogen-free facility at UCSF. All procedures were performed in concordance with UCSF Institutional Animal Care and Use Committee (IACUC) regulations and approved protocol. Timed-pregnant C57BL/6 females were purchased from Jackson Laboratories (strain #000664). Retro-orbital (RO) injections were done following with UCSF IACUC procedural guidelines. Briefly, pregnant dams were anesthetized at E13.5-E15.5 with isoflurane and injected in the RO sinus using 0.5 mL insulin syringes, with a maximum volume of 150 μL containing 5 μg of human IL1B (Peprotech 200-01B) and 50 μg of polyclonal goat IgG (isotype control; R&D Systems AB-108-C) or of goat anti-human IL1RA antibody (R&D Systems AF-280-NA) in sterile PBS. Reagents were mixed immediately prior to injection.

### J. Mouse Harvesting

Mice were euthanized at E18.5 with CO₂, and a sample of serum was obtained via transcardial puncture. Resorbed and malpefrused fetuses were quantified. Whole fetuses and their placentas were harvested and weighed. Murine IL1B concentrations were measured in maternal serum by a commercial ELISA (Abcam Cat No. ab 197742). Cephalic vasculature was quantified from images of dissected fetuses using the Vessel Analysis plug-in for ImageJ software.

### K. Bioinformatic Analyses

To analyze peptide enrichment after PhIP-seq, reads were aligned at the protein level using RAPSearch (Zhao et al. “RAPSearch2: a fast and memory-efficient protein similarity search tool for next-generation sequencing data” Bioinformatics, 28 (1): 125-126 (2012); Ye et al. “RAPSearch: a fast protein similarity search tool for short reads.” BMC Bioinformatics 12:159 (2011)). Aligned reads were normalized to 100,000 reads per k-mer (RPK) to account for varying read-depth and further normalized to the mean of either term or preterm sera within each cohort and batch. Post-normalization batch effect was not observed. Autocorrelation between technical replicated samples and individual samples was evaluated by Pearson correlation. Preterm- or term-specific candidate antigens were identified using an implementation of PhagePy python package. Briefly, a log-fold changes were calculated over mean term or preterm samples and z-scores were derived to identify enriched peptides within each individual. Peptides with z-scores greater than or equal to 3 were considered hits and either preterm- or term-specific peptides were identified by filtering by requiring that the peptide was a hit in at least 2 cases and zero controls/Ridge regularized logistic regression was utilized to derive preterm birth classifiers using fold changes over mock IP or raw placental IP-mass spectrometry data. Unsupervised clustering was performed using UMAP implementation in python (McInnes et al. “UMAP: Uniform Manifold Approximation and Projection for Dimension Reduction” ArXiv e-prints 1802.03426 (2018)).

### Initial Investigation

### Example 2: Global Remodeling of Antibody Specificity During Pregnancy

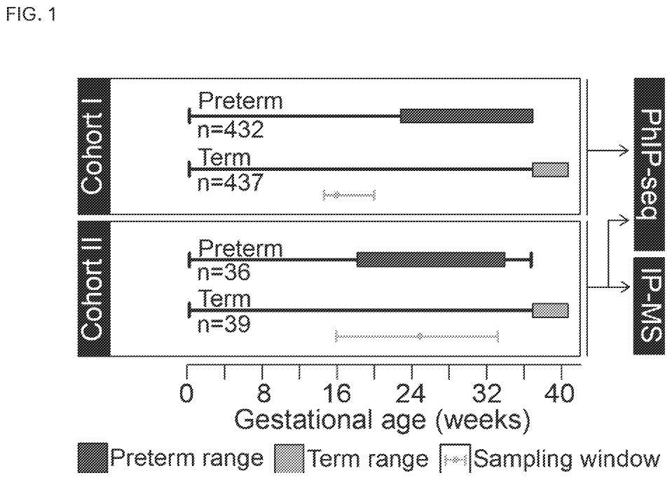

Pregnancy induces immune tolerance to the semi-allogeneic fetus, yet the capacity to generate protective antibodies to pathogens remains robust to passively immunize the offspring. The maintenance of these protective mechanisms may incompletely disarm humoral responses leading to antibody generation to fetal antigens, which are shed from the placenta. To interrogate this, a PhIP-seq library of peptides tiling the human proteome was used to pan serum from a large, cross-sectional, population-wide cohort of maternal sera from singleton pregnancies randomly selected from a California-wide biobank, balancing between term and preterm outcomes (CA1000 cohort; n=450 term, n=447 preterm, mean 16.6 weeks gestation at sampling; detailed data not shown, see Table 1 and FIG. 1 of U.S. Provisional Application No. 63/564,699) and a later gestation case-control cohort of deliveries complicated by preterm premature rupture of membranes (PPROM) or matched healthy term pregnancies (PPROM cohort; n=25 term, n=20 preterm, mean 25.9 weeks gestation at sampling; detailed data not shown, see Table 2 and FIG. 1 of U.S. Provisional Application No. 63/564,699). Samples from healthy individuals not currently pregnant were drawn from a New York Blood Center cohort (Healthy Controls; n=100; n=49 male, n=51 female; detailed data not shown, see Table 3 and FIG. 1 of U.S. Provisional Application No. 63/564,699) and served as baselines.

Utilizing high-throughput methods, three rounds of enrichment were performed on 1,016 samples from the discovery cohort and healthy controls in a single experimental run. The expected peptides were highly enriched with a polyclonal control antibody specific to GFAP (FIG. 16). Immunoprofiles from healthy controls did not differ across experimental plates or days (data not shown, see Table 7 of U.S. Provisional Application No. 63/564,699) and technically replicated samples (n=27) exhibited high co-correlation (data not shown, see Table 8 and FIG. 1 of U.S. Provisional Application No. 63/564,699) indicating the technical stability of PhIP-seq technique.

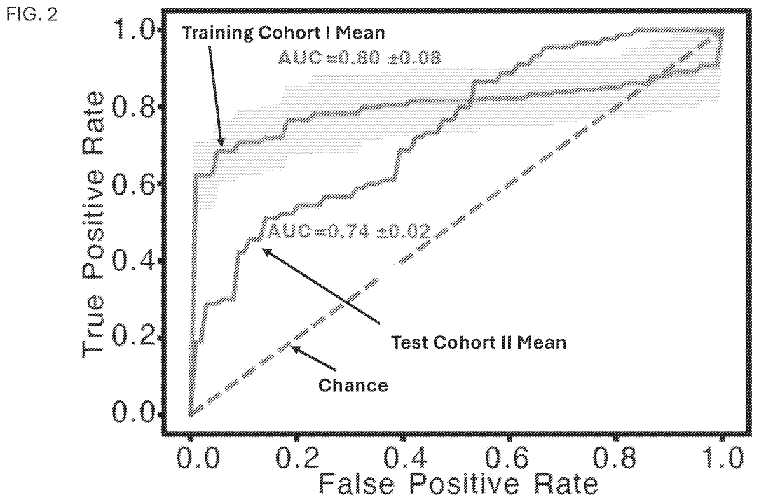

Co-correlation analysis of global PhIP-seq signatures was first used to detect whether differences between preterm and term outcomes were present early in gestation within the CA1000 cohort. Healthy non-pregnant people from the NYBB cohort exhibited a highly individualized signature by PhIP-seq and co-correlation coefficients greater than 0.5 were not detected. A mean degree of connectivity was derived by calculating the proportion of individuals sharing a correlation coefficient greater than 0.3, which resulted in a mean degree of connectivity of 0.03 in healthy non-pregnant people (data not shown, see FIG. 2 of U.S. Provisional Application No. 63/564,699). The sera obtained from pregnant subjects exhibited a complete shift in antibody profile, increasing the mean degree of sharing across individuals by 6-fold (data not shown, see FIG. 2 of U.S. Provisional Application No. 63/564,699) in term pregnancies uncomplicated by medical or environmental factors (term, uncomplicated; n=150; see Methods). One or more complications in term pregnancies elevated the degree of sharing by 1.5-fold relative to healthy term pregnancies (term, complications; n=234). Antibody co-correlation and sharing amongst individuals was most pronounced in preterm pregnancies with a 14-fold greater mean degree of sharing relative to healthy non-pregnant controls and 2.4-fold increase above healthy term pregnancies (n=447; data not shown, see FIG. 2 of U.S. Provisional Application No. 63/564,699).

Given that gravidity may influence experience to placental and paternal antigens that persists beyond pregnancy, co-correlation of antibody profiles was next compared between primo- and multi-gravida individuals. In healthy term pregnancies, the mean degree of connectivity was similar to non-pregnant individuals in primigravida, and this connectivity was 4-fold greater in multigravida (data not shown, see Table 5 of U.S. Provisional Application No. 63/564,699). Complications, including preterm delivery, resulted in increased connectivity between primigravida and was the greatest in preterm multigravida (data not shown, see Table 5 of U.S. Provisional Application No. 63/564,699). Repeated pregnancies possibly expand the antigenic landscape. The data discussed in this disclosure points to a dosage dependent effect of pregnancy on antibody specificity, which is consistent with the known associations of poor obstetric history and preterm birth.

### Example 3: Maternal Autoreactive Antibody Signatures Predict Preterm Delivery

Peptide-level PhIP-seq enrichments across the entire proteome were collapsed to the protein-level and used to train a machine learning classifier in the CA1000 cohort. Using all features, preterm outcome was predicted with a positive predictive value of 97% and a negative predictive value of 100% with a receiver operating characteristic (ROC) plot area under ROC curve (AUC) of 0.96 in a leave-one-out cross-validation test (data not shown, see FIG. 2 of U.S. Provisional Application No. 63/564,699). This classifier was tested in the PPROM cohort as an independent validation cohort and resulted in a lower predictive ability with an ROC AUC of 0.67 (data not shown, see FIG. 17 of U.S. Provisional Application No. 63/564,699).

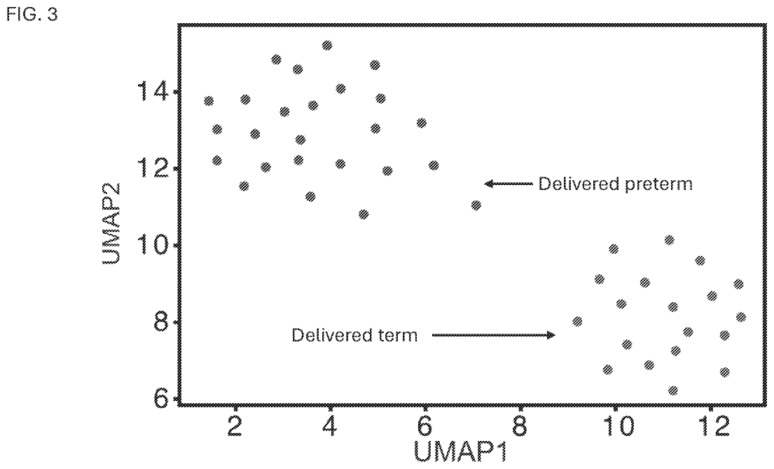

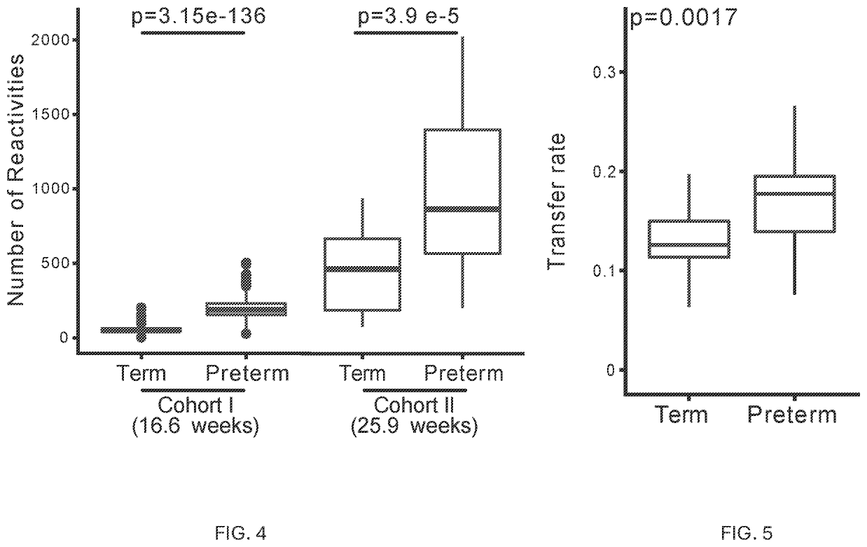

To identify antibody specificities enriched in preterm pregnancy that contribute to preterm predictive ability, log-transformed fold changes over term, uncomplicated pregnancies were calculated for peptide PhIP-seq reads per 100,000 (RPK; data not shown, see FIG. 3 of U.S. Provisional Application No. 63/564,699). A peptide was defined as preterm-specific using a z-score cut-off of 3 above healthy term pregnancy and present in at least two preterm and no term pregnancies (data not shown, see FIG. 3 of U.S. Provisional Application No. 63/564,699). This stringent filtering yielded around 10,000 preterm antibody specificities at the protein level in the CA1000 and the PPROM cohorts (data not shown, see FIG. 4 of U.S. Provisional Application No. 63/564,699). The approach was reversed to identify term-specific peptides by calculating log-transformed fold changes over preterm samples (data not shown, see FIG. 3 of U.S. Provisional Application No. 63/564,699). This resulted in 3-fold fewer peptides than in preterm pregnancies in the CA1000 cohort (data not shown, see FIG. 4 of U.S. Provisional Application No. 63/564,699), indicating that preterm pregnancy exhibits greater autoreactivity at early gestation. Greater than 55% of antibody reactivities were shared between CA1000 and PPROM cohorts, generating a list of reactivities that were present at 16 and 25 weeks of gestation in two distinct preterm birth cohorts (data not shown, see FIG. 5 of U.S. Provisional Application No. 63/564,699). Sharing of reactivities was less than 30% among term pregnancy hits, indicating that the majority of term-specific peptides are gestational-age specific or related to other population differences between these two cohorts (data not shown, see FIG. 5 of U.S. Provisional Application No. 63/564,699).

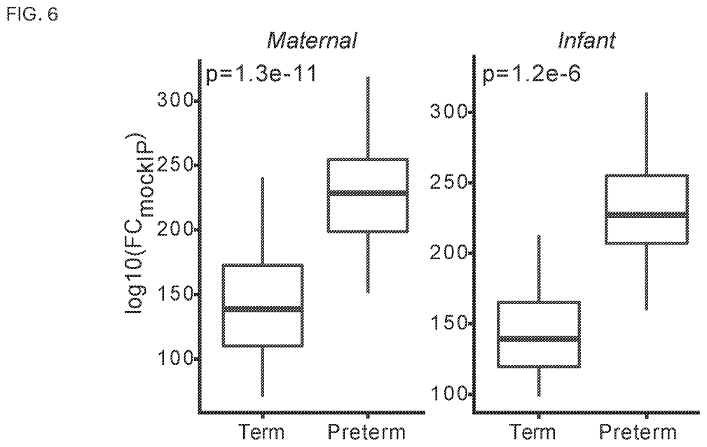

Preterm-specific antibody reactivities exhibited convergence among key pathways, such as the NFkB pathway, immune-related, myosin, placental, and developmental proteins (data not shown, see Table 7 of U.S. Provisional Application No. 63/564,699). Among these reactivities, several proteins associated with preterm delivery were identified. For example, antibodies to inflammasome related proteins were enriched in subsets of preterm pregnancies in the CA1000 cohort such as IL1 receptor antagonist (IL1RA) and its paralog, IL36RA, as well as NLRP12 (data not shown, see FIG. 6 of U.S. Provisional Application No. 63/564,699). Proteins highly expressed on placenta (SIGLEC8, STOX1, and SVIL) were also targeted by antibodies in preterm pregnancies (data not shown, see FIG. 6 of U.S. Provisional Application No. 63/564,699). Likewise, developmental proteins, such as embryonic lethal NSUN2 and P4HTM and hormone receptors RORA and follicular stimulating hormone receptor (FSHR) were enriched in preterm pregnancies. These data indicated that distinct pathways required for the maintenance of pregnancy were targeted by maternal antibodies in preterm pregnancies.

### Example 4: Maternal Antibodies Bind Placental Proteins

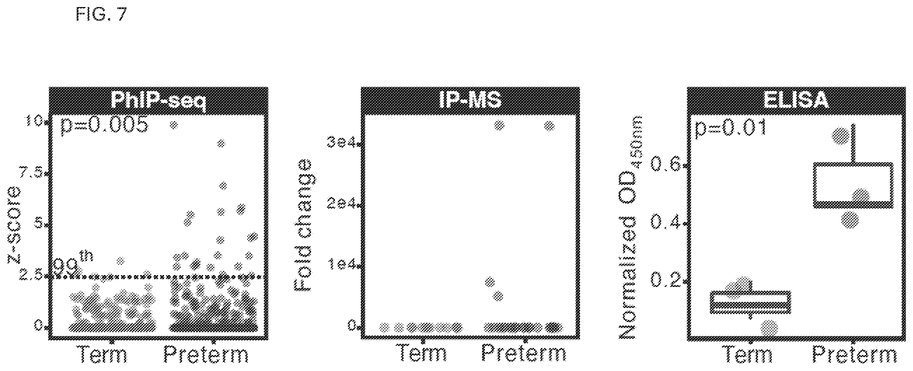

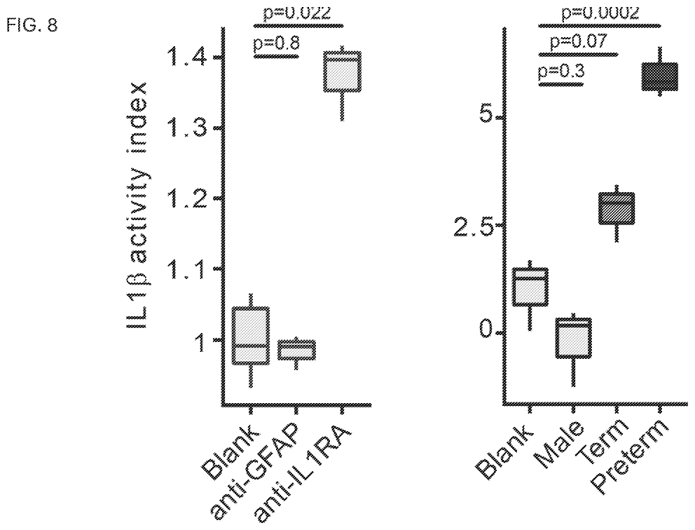

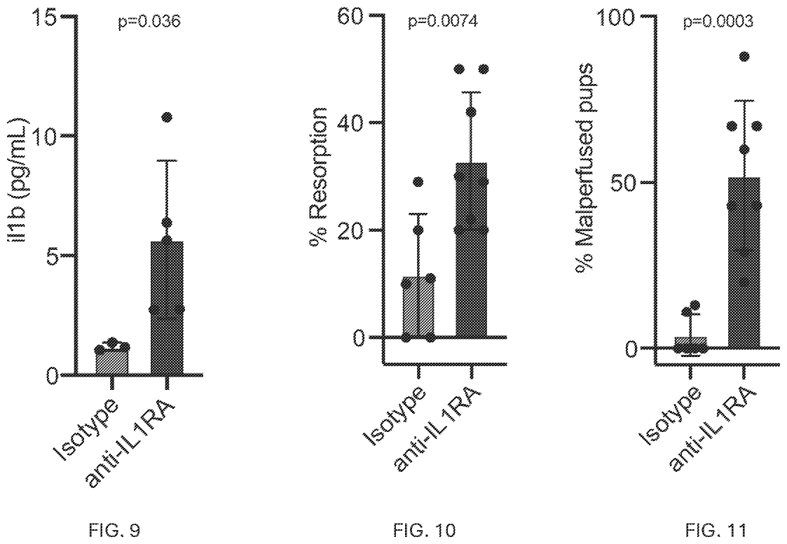

Since PhIP-seq results indicated that placental proteins may be targeted by maternal antibodies in preterm pregnancies, a native immunoprecipitation with sera from the PPROM cohort was performed using placental protein lysate, and the antibody targets were identified by untargeted mass spectrometry (n=19 term, n=25 preterm; data not shown, see FIG. 7 of U.S. Provisional Application No. 63/564,699). Preterm-specific antibody reactivities were identified by calculating log-fold changes over term controls, and a threshold of z-score greater than three was used to find preterm-specific proteins (data not shown, see FIG. 7 of U.S. Provisional Application No. 63/564,699). A total of 305 proteins was identified, and 71% of these were also found by PhIP-seq, orthogonally validating these antibody reactivities (data not shown, see FIG. 7 and Table 6 of U.S. Provisional Application No. 63/564,699). Reactivities identified by placental IP-MS were highly discriminatory between preterm and term outcomes by unsupervised UMAP clustering (data not shown, see FIG. 8 of U.S. Provisional Application No. 63/564,699). Among the top 50 reactivities by IP-MS were key reactivities identified by PhIP-seq, including IL1RA (data not shown, see FIG. 9 and FIG. 10 of U.S. Provisional Application No. 63/564,699). A 3-fold validated lasso-regularized logistic regression classifier was trained using placental IP-MS data and performed with excellent classification ability (ROC AUC 0.91±0.05; data not shown, see FIG. 11 of U.S. Provisional Application No. 63/564,699).

### Example 5: Maternal Anti-IL1RA Functionally Inhibit IL1B Antagonism

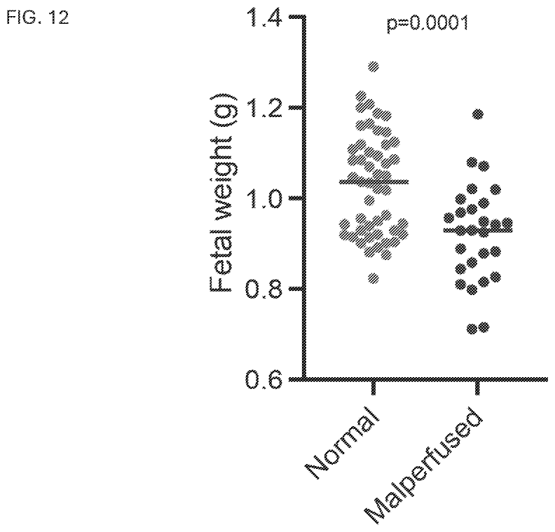

Single nucleotide polymorphisms in IL1RA are associated with PPROM and intrauterine injection of IL1B results in extreme prematurity in Rhesus macaques. indicating that limiting IL1 signaling is necessary and sufficient to induce preterm labor. Maternal anti-IL1RA antibodies were identified in 18% and 16% of preterm pregnancies in the CA1000 by PhIP-seq and PPROM cohorts by placental IP-MS, respectively. Epitopes on IL1RA were further investigated, as both methods afford amino acid-level resolution of antibody targets. All preterm anti-IL1RA antibodies in the PPROM cohort targeted the epitope LQLEAVNITDLSENRK (SEQ ID NO:2) as identified by placental IP-MS, which was also the preterm-specific epitope in the CA1000 PhIP-seq experiments (data not shown, see FIG. 12 of U.S. Provisional Application No. 63/564,699). Structural analysis of crystalized IL1RA bound to the IL receptor indicated that this epitope was within a highly allipathic unstructured loop opposite the receptor engaging face (data not shown, see FIG. 13 of U.S. Provisional Application No. 63/564,699). Additionally, the anti-IL1RA epitope spans the sole glycosylation site (Asp109) as well as a terminal lysine (Lys118) that has been shown to be required for IL1RA polymerization (data not shown, see FIG. 13 of U.S. Provisional Application No. 63/564,699). Indirect immunofluorescence with commercial anti-IL1RA antibodies in placenta revealed a striking filamentous pattern that co-localized within CD31 endothelial vessels (data not shown, see FIG. 14 of U.S. Provisional Application No. 63/564,699). Pre-incubation of antibody with recombinant IL1RA prior to staining completely abrogated the filamentous pattern, indicating that filaments are likely IL1RA polymers.

The above data indicated that anti-IL1RA antibodies functionally inhibited IL1B antagonism, contributing to the inflammatory environment that precipitated preterm labor. An IL1 reporter cell line was used to measure the ability of anti-IL1RA to rescue IL1B signaling after incubation with recombinant IL1RA in vitro. Sera from anti-IL1RA positive mothers or term sera was pre-incubated with recombinant IL1RA and added to an ILI receptor reporter cell line after stimulation with IL1B. Percent IL1 recovery was calculated for the sera from both term and preterm individuals from the PPROM cohort. IL1RA antagonism was inhibited with anti-IL1RA positive preterm sera, and increased IL1B reporter activity was observed, in comparison to anti-IL1RA negative term sera (data not shown, see FIG. 15 of U.S. Provisional Application No. 63/564,699). Together, these data indicated that maternal anti-IL1RA antibodies defined a significant subset of preterm pregnancies and that these antibodies were capable of functionally inhibiting IL1B antagonism.

### Updated Investigation

### Example 6: Updated Study of Global Remodeling of Antibody Specificity During Pregnancy

Pregnancy induces immune tolerance to the semi-allogeneic fetus, yet the capacity to generate protective antibodies to pathogens remains robust to passively immunize the offspring. The maintenance of these protective mechanisms may incompletely disarm humoral responses leading to antibody generation to fetal antigens, which are shed from the placenta. To investigate autoreactivity during pregnancy, maternal sera from term and preterm pregnancies were used to determine antibody specificity in two cross-sectional pregnancy cohorts, and a comparison was made to non-pregnant controls. Sera from these three cohorts was used to immunoprecipitate a customized, previously validated phage display library of the human proteome with (PhIP-seq) approach (O'Donovan et al. “High-resolution epitope mapping of anti-Hu and anti-Yo autoimmunity by programmable phage display.” Brain Communications 2:1-16 (2020)). The first population-wide cohort of maternal sera was assembled from randomly selected maternal sera collected at collected at mean 16.5 weeks of gestation from a California-wide biobanking effort, with comparable numbers of preterm and term pregnancy outcomes and reflective of state demographics (Cohort I; n=869; n=437 term, n=432 preterm). A secondary cohort was collected at a mean of 26.1 weeks of gestation from women experiencing spontaneous preterm labor complicated by PPROM or control pregnancies that delivered at term (Cohort II; n=75; n=39 term, n=36 preterm). Samples from healthy non-pregnant individuals served as additional controls (Baseline Cohort; n=100; n=49 male, n=51 female).

Using a high-throughput method (Vazquez et al. “Autoantibody discovery across monogenic, acquired, and COVID-19-associated autoimmunity with scalable PhIP-seq.” eLife 11: e78550 (2022)), PhIP-seq was performed with 1 μL of serum on a total of 1,407 samples. Following three rounds of panning, phage genomic inserts were sequenced and aligned to the input library to calculate peptide enrichments. A commercial anti-glial fibrillary acidic protein (GFAP) polyclonal antibody was used as a positive control and peptides tiling across GFAP were approximately 2×10⁵-fold more enriched in control immunoprecipitations (IPs) than IPs with sera from the human cohort samples. Prior to any filtering, PhIP-seq immunoprofiles from healthy controls were investigated for batch effects. Peptide-level RPK PhIP-seq profiles did not differ across experimental batches by Uniform Manifold Approximation and Projection (UMAP) analysis, and a subset of samples that were technically replicated across experimental batches (n=27) exhibited high Pearson co-correlation. A total of 293 significantly enriched peptides were identified in mock IPs and filtered from downstream analysis.

Peptide-level fold change relative to mock IP controls was calculated, and a co-correlation analysis was performed to detect global differences amongst healthy controls, preterm, and term pregnancies in Cohorts I and II. Using a Pearson correlation threshold of 0.3, the number of co-correlating pairs for each group of subjects was determined and expressed as “percent connectivity” (the number of connected subjects as a percentage of the total). Among non-pregnant controls, the percent connectivity was 22.9% (22 of 96), consistent with previous observations that PhIP-seq signatures are highly individualized (Larman et al. “PhIP-Seq characterization of autoantibodies from patients with multiple sclerosis, type 1 diabetes and rheumatoid arthritis.” Journal of Autoimmunity 43:1-9 (2013)). Percent connectivity among term uncomplicated pregnancies (n=208) was 20.2% and the distribution of correlation coefficients was significantly greater from non-pregnant controls (p adj=5.4e-44), suggesting additional sharing of autoreactivity related to pregnancy itself. While term pregnancies with complications exhibited a slight increase percent connectivity (26.2%, n=229, p adj=3.4e-30), the largest increase was observed in preterm pregnancies (40.0%, n=432, p adj-3.5e-143). These results suggested that complications and preterm pregnancies elicited a greater degree of autoreactive antibody sharing than term pregnancies or non-pregnant individuals.

Given that B cell memory to fetal antigens likely persists beyond gestation (Suah et al. “Pregnancy-induced humoral sensitization overrides T cell tolerance to fetus-matched allografts in mice.” Journal of Clinical Investigation 131 (2021); Porrett “Biologic mechanisms and clinical consequences of pregnancy alloimmunization.” American Journal of Transplantation 18:1059-1067 (2018)), the influence of gravidity (total number of pregnancies) on maternal autoantibody profiles was investigated. Among primigravida, percent connectivity was increased among preterm pregnancies relative to term (12.5% vs 57.1%). Among multigravida, a similar significant increase in percent connectivity was also observed (18.1% vs. 33.4%). Greater percent connectivity was also noted in spontaneous rather than iatrogenic preterm pregnancies. These results indicated that the increase in autoreactive profile sharing was most pronounced in spontaneous preterm pregnancies and was independent of gravidity in preterm, but not term pregnancies.

### Example 7: Maternal Autoreactive Antibody Signatures Precede and Predict Preterm Delivery

The presence of increased autoreactive antibody sharing among preterm pregnancies as early as 16.6 weeks of gestation and preceding preterm delivery suggested that these data may be used to build predictive classification models. For an initial training set, Cohort I was divided into the subsets of uncomplicated term pregnancies (n=216) and spontaneous preterm pregnancies. Iatrogenic preterm pregnancies (n=91) were excluded as were term pregnancies with complications. A five-fold stratified cross-validated lasso-regularized logistic regression model was built using fold-change over healthy non-pregnant controls as input. Feature selection was performed by requiring that a peptide was enriched in at least five preterm pregnancies and zero term pregnancies within the training set of each cross-validation fold. Model performance was evaluated by receiver-operator curve (ROC) analysis, yielding a mean area under the curve (AUC) of 0.8±0.08 across all cross-validation folds (FIG. 2). Analysis of top peptides by logistic regression coefficient in Cohort I revealed a highly specific, though sparse signal that preceded preterm delivery by 15 weeks. As an independent validation, the trained model was used to predict on PhIP-seq data from all samples in Cohort II, which were never included in the training phase. In this completely independent cohort, model performance yielded a mean AUC of 0.74±0.02 (FIG. 1).

IP-MS was used as a complementary technique to identify whole protein targets. A subset of sera from Cohort II (n=19 term, n=25 preterm) was randomly selected and used to immunoprecipitate native proteins from placental extract. Antibody targets were identified using trapped ion mobility time of flight mass spectrometry (timsTOF-MS). Unsupervised machine learning analysis of the enriched protein counts yielded two clear clusters, distinctly segregated preterm or term samples (FIG. 3). Using the same mass spectrometry-derived protein counts, a three-fold stratified cross-validated lasso-regularized logistic regression model was constructed (see Methods). Testing on a third of the data, held back from training, yielded models with a mean AUC of 0.88±0.11. Top ranked proteins by logistic regression coefficient revealed sharing across multiple individuals, and hierarchical clustering of these features clearly separated preterm versus term sera (Table 1). These data and positive AUC values for both Cohort I and II support the notion that a multitude of early pregnancy serological indicators are predictive of spontaneous preterm delivery.

### Example 8: Identification of Autoreactivities that Define Spontaneous Preterm Pregnancies

To identify both preterm and term specific autoreactivities, a conservative analysis pipeline was implemented. Z-scores were first calculated using the mean of enrichment values for healthy term pregnancies. Peptides with a z-score of 3 or greater were designated as pre-term specific if they were shared by at least two preterm subjects and were never significantly enriched in healthy term samples (z-score≤3). Conversely, term specific peptides were identified using the mean of the pre-term samples to calculate z-scores. Across both cohorts, the number of autoreactivities was significantly higher in preterm versus term pregnancies and the number of reactivities appeared to increase with advancing gestation (FIG. 4). Preterm autoantibody targets were highly shared across Cohort I and Cohort II, with 6,926 overlapping proteins. Term proteins exhibited lower overlap between these distinct cohorts, with only 2,642 overlapping proteins.

The preterm-specific autoantibody signature in PhIP-seq data was compared to enrichments in placental IP-MS data from Cohort II. Preterm-specific reactivities were identified by calculating log-fold changes over term controls and utilizing a threshold of z-score greater than three. A total of 343 proteins were identified and 65% of these were also found by PhIP-seq.

### Example 9: Preterm Autoantibody Signature is Transferred from Mother to Infant

Maternal antibodies are transferred to the fetus by transcytosis across the placenta and provide passive immunity to the infant within the first four months of life. However, it is know that not all maternal antibodies are transferred, especially those that are autoreactive (Innes, A., Stewart et al. “Human placenta—an antibody sponge?” Am J Reprod Immunol Microbiol 17:57-60 (1988); Dolatshahi et al. “Selective transfer of maternal antibodies in preterm and fullterm children.” Sci Rep 12:14937 (2022); Jennewein et al. “Fc Glycan-Mediated Regulation of Placental Antibody Transfer. Cell 178:202-215.e14 (2019)). To investigate the degree of maternal autoantibody transfer to the fetus, two additional cohorts with paired maternal and cord blood serum were analyzed by PhIP-seq. Cohort III was comprised of n=15 mother-infant dyads from term pregnancies, while Cohort IV was comprised of n=64 dyads from preterm pregnancies. Co-correlation analysis of PhIP-seq autoantibody profiles revealed that all mother-infant dyads from term and preterm pregnancies exhibited high co-correlation (mean Pearson correlation coefficients 0.8 and 0.9, respectively), though this was slightly elevated in preterm pregnancies.

To investigate specific autoreactivities shared within dyads, peptides above the z-score threshold of 3 relative to mock IPs were counted in mother-infant pairs. Given that newborns lack the capacity to produce IgG antibodies at birth, it was inferred that these shared autoreactivities were transferred from the mother. Next, the transfer ratio, defined here as the proportion of maternal autoreactivities that were transferred in each dyad, was calculated by dividing number of transferred reactivities by the number of maternal reactivities for each dyad. In both term and preterm pregnancies, transfer rate was <1 (FIG. 5), indicating inhibitory mechanisms of autoantibody transfer compared to expected >1.5 transfer rate for vaccine antibodies observed in term pregnancies (Atyeo et al. “COVID-19 mRNA vaccines drive differential antibody Fc-functional profiles in pregnant, lactating, and nonpregnant women.” Science Translational Medicine 13 (2021)). The autoreactive transfer ratio was significantly higher in preterm versus term pregnancies (FIG. 5), likely reflecting the higher autoreactivity that defines these pregnancies.

Next, the inter-dyad relationships were investigated. As predicted by the highly individualized PhIP-seq signature, maternal inter-dyad correlations were significantly lower. Using the spontaneous preterm pregnancy-specific reactivities identified in Cohort I, the cumulative enrichment of autoreactivities in Cohort IV (preterm) was significantly higher than Cohort III (term) (FIG. 6). A co-correlation analysis was performed between maternal PhIP-seq enrichments (inter-dyad) utilizing only the preterm-specific reactivities identified in Cohort I as input. Inter-dyad co-correlations with a correlation coefficient greater than 0.3 were significantly increased from 13% to 73% in preterm pregnancies. Similar inter-dyad correlation trends were observed in infant-infant or maternal-infant pairs. Thus, preterm reactivities identified in Cohort I are also enriched in a third cohort of preterm pregnancies (Cohort IV) but not in term pregnancies (Cohort III).

A list of reactivities transferred in preterm pregnancies was identified by requiring that a reactivity was transferred in at least three preterm dyads in Cohort IV. This resulted in reactivities targeting 10259 proteins, 76% of which were also identified as preterm-specific in Cohort I by PhIP-seq. When compared to the short-list of proteins identified as preterm-specific in Cohort II by IP-MS (343 proteins), 152 of these proteins (or 44%) were also transferred and enriched in Cohort I by PhIP-seq. This represented a conservative list of validated, preterm-specific, transferred reactivities (Table 2).

### Example 10: Targets within the Preterm Autoantibody Signature

Autoantibodies can directly influence the function of their target proteins, thereby leading to functional defects. Patterns of autoantibody targets within the preterm-specific signature were investigated using pathway gene-set enrichment analysis. Significantly enriched pathways (adjusted p-value <0.05; 26 pathways) were manually grouped by their shared features. These included hormone synthesis and signaling, vascular cell development and function, muscle contractility, cell proliferation, and infection response categories.

The placenta is a source of hormone production essential for the successful maintenance of pregnancy (Napso et al. “The Role of Placental Hormones in Mediating Maternal Adaptations to Support Pregnancy and Lactation.” Frontiers in Physiology 9 (2018)). Interference or dysregulation of these pathways may lead to pregnancy complications or spontaneous preterm labor. Several critical reproductive hormones (and/or their receptors) were significantly enriched in the preterm autoantibody signature. These included progesterone and estrogen receptors (PGR, ESR1, ESR2), prolactin (PRL, PRLR), human placental lactogen (CSH2), gonadotropins (CGA, GNRH2, GNRHR), follicular stimulating hormone receptor (FSHR), and relaxin receptors (RFXP1, RFXP3). Thus, preterm autoantibody specificities targeting reproductive hormones may contribute to a broader hormonal dysfunction observed in preterm pregnancies (Vidal et al. “Spontaneous preterm birth: Involvement of multiple feto-maternal tissues and organ systems, differing mechanisms, and pathways.” Frontiers in Endocrinology 13 (2022)).

Sterile and infectious inflammation are the most frequently identified causes of spontaneous preterm labor (Goldenberg et al. “Epidemiology and causes of preterm birth. Lancet (London, England) 371:75-84 (2008)). Preterm autoantibody targets in the infection response category included histocompatibility proteins (HMHA1, HLA-A, HLA-B, HLA-C, HLA-DMA, HLA-DOA, HLA-DOB, HLA-F, HLA-E) and the placental-specific HLA-G. Anti-HLA antibodies increase the risk of spontaneous preterm delivery (and are associated with complement deposition in the placental endothelium, which occurs more frequently in spontaneous preterm labor (Lee et al. “Detection of anti-HLA antibodies in maternal blood in the second trimester to identify patients at risk for antibody-mediated maternal anti-fetal rejection and spontaneous preterm delivery.” Am J Reprod Immunol 70:162-175 (2013)). Anti-cytokine antibodies, known to increase susceptibility to infection (Bastard et al. Autoantibodies against type I IFNs in patients with life-threatening COVID-19. Science 370 (2020)), were also identified. Of these, anti-IL1RA and anti-IL16 were the most significantly enriched. Anti-IL1RA was the only anti-cytokine reactivity that was present in all three preterm pregnancy cohorts both by PhIP-seq and IP-MS and was also transferred from mother to fetus.

### Example 11: Neutralizing Anti-IL1RA Autoantibodies Define a Subset of Spontaneous Preterm Pregnancies

Given the established role of IL IB for the promotion of labor (Sadowsky et al. “Preterm labor is induced by intraamniotic infusions of interleukin-1β and tumor necrosis factor-α but not by interleukin-6 or interleukin-8 in a nonhuman primate model.” American Journal of Obstetrics and Gynecology 195:1578-1589 (2006)), finding the secreted competitive antagonist, IL1RA, as a consistently enriched autoreactive target was surprising. Antibodies to IL1RA, encoded by the ILIRN gene, were investigated. The prevalence of anti-IL1RA positive preterm pregnancies was 9.5% in Cohort by PhIP-seq, and 20% of preterm sera by IP-MS in Cohort II (FIG. 7). Sera remaining from three out of four anti-IL1RA positive Cohort II samples were tested in a native ELISA with supernatants from IL1RA over-expression in HEK293T cells. Sera from these preterm pregnancies resulted in significantly greater ELISA signal compared to known anti-IL1RA negative term pregnancies (FIG. 7). Next, using a commercial antibody to IL1RA (Sigma Prestige HPA001482), placental localization of IL1RA was investigated by indirect immunofluorescence analysis. A striking pattern of IL1RA localization was found near CD31-positive fetal vessels within the placenta that also co-localized with VE-cadherin in endothelial cell junctions. While IL1RA is a known secreted protein, these results suggest a substantial amount of this protein is localized to the endothelial adherent junctions in the placenta. To address whether the observed pattern of co-localization was authentic, or due to cross reactivity of the antibody with other targets, PhIP-seq was performed with the same antibody and analyzed for enrichment. Only IL1RA protein was enriched above background, suggesting the commercial IL1RA antibody is highly specific. As a further validation of antibody specificity, a blocking assay was performed with the peptide immunogen used to generate the antibody. The signal corresponding to IL1RA was eliminated, again suggesting this antibody is highly specific. Together, these data support the notion that IL1RA is bound at placental endothelial cell junctions, in close proximity to other junction proteins, like VE cadherin.

Using the approach similar to the one described above, it was investigated whether anti-IL1RA⁺ patient sera were capable of blocking IL1RA on placental tissue. Placenta was first blocked with either PBS or term or anti-IL1RA⁺ patient sera, and then indirect immunofluorescence was performed with the commercial anti-IL1RA antibody. While the characteristic pattern of IL1RA localization to fetal vessel cell-cell junctions was observed in PBS, term sera or anti-IL1RA preterm sera treated conditions, this pattern was blocked in placental tissue treated with anti-IL1RA⁺ preterm sera. These data strongly supported the presence of highly specific autoantibodies in the maternal sera of a subset of preterm pregnancies and suggests a mechanism in which the natural role of the IL1RA antagonist is subverted, potentially leading to labor-inducing levels of IL1B.

To identify the epitope on IL1RA targeted by anti-IL1RA⁺ pregnancies, peptide fragments from PhIP-seq and IP-MS were analyzed. Positional mean enrichment analysis of PhIP-seq data revealed that the majority of anti-IL1RA⁺ pregnancies targeted overlapping peptides between D99-D129 amino acid residues on IL1RA. This amino acid stretch, opposite the receptor engaging face, is highly charged, and contains the only N-linked glycosylation residue in the protein-N109 (Schreuder et al. “A new cytokine-receptor binding mode revealed by the crystal structure of the IL-1 receptor with an antagonist. Nature 386:194-200 (1997); Krishnan et al. “Multistep Aggregation Pathway of Human Interleukin-1 Receptor Antagonist: Kinetic, Structural, and Morphological Characterization.” Biophysj 96:199-208 (2009)). Antibodies to the epitopes contained in the above amino acid stretch have previously been reported in IgG4 disease (Jarrell et al. “Neutralizing anti-IL-1 receptor antagonist autoantibodies induce inflammatory and fibrotic mediators in IgG4-related disease.” J Allergy Clin Immunol 149:358-368 (2022)), and it is also targeted by two commercial antibodies. IP-MS fragment analysis identified a single 16 amino acid peptide LQLEAVNITDLSENRK (SEQ ID NO:2; amino acids 103-118 of SEQ ID NO:1) that was fully contained within the target PhIP-seq fragment. These data confirmed that the target epitopes enriched by both PhIP-seq and IP-MS are localized to the same region, and that anti-IL1RA antibodies present in multiple pre-term sera converge on the same region of the protein.

To investigate the functional consequences of anti-IL1RA antibodies, a previously described IL1B reporter cell line and validated assay was utilized (Jarrell et al. “Neutralizing anti-IL-1 receptor antagonist autoantibodies induce inflammatory and fibrotic mediators in IgG4-related disease. J Allergy Clin Immunol 149:358-368 (2022); Thurner et al. “IL1RA Antibodies in Myocarditis after SARS-COV-2 Vaccination. N Engl J Med 387:1524-1527 (2022); Pfeifer et al. “Autoantibodies against interleukin-1 receptor antagonist in multisystem inflammatory syndrome in children: a multicentre, retrospective, cohort study.” The Lancet Rheumatology 4: e329-e337 (2022)). Briefly, neutralization of IL1RA was measured using a reporter cell line in which ILI receptor activity results in secreted embryonic alkaline phosphatase (SEAP). Reporter cells were stimulated with recombinant IL1B and a titration of increasing amounts of excess of IL1RA and serum or antibody, SEAP rates were normalized to the negative control (TNFα treated cells) and an IL1B activity index was calculated for cells stimulated with IL1RA and IL1B relative to IL1B alone. Compared to cells treated with PBS or off-target anti-GFAP antibody, a commercial antibody (R&D Systems AF-280-NA) that primarily targets the patient epitope, resulted in significant neutralization of IL1RA as measured by greater IL1B activity (FIG. 8). Similarly, anti-IL1RA⁺ preterm sera also resulted in significantly higher IL1B activity (FIG. 8). Conversely, cells treated with PBS, male, or term sera exhibited low IL1B activity. Thus, both commercial antibodies and the antibodies from the preterm labor patients were sufficient to neutralize IL1RA antagonist, thus permitting uninhibited IL1B signaling.

### Example 12: Maternal Anti-IL1RA Antibodies Promote Fetal Resorption and Malperfusion In Vivo

To further explore the physiological consequence of anti-IL1RA antibodies in vivo, the role of maternal anti-IL1RA antibodies was investigated during murine pregnancy using that same commercial anti-IL1RA antibody that was found to block the antagonist. First, it was confirmed by dot blot that the antibody was cross-reactive to the native mouse orthologue by dot blot. Timed-pregnant dams were injected intravenously three times (Embryonic Days 13.5-15.5; E13.5-15.5) with either the anti-IL1RA antibody or an equivalent amount of isotype control in the context of IL 1B-induced inflammation. Levels of murine IL1B were measured in maternal serum by ELISA on the day before expected delivery (E18.5). IL1B levels were significantly higher in anti-IL1RA injected dams as compared to isotype controls (FIG. 9), indicating that anti-IL1RA neutralizes the ability of the antagonist to regulate IL1B during pregnancy. Anti-IL1RA injected dams also exhibited profound reproductive defects. Resorption rate was significantly elevated in anti-IL1RA-injected dams as compared to isotype (FIG. 10). Among the non-resorbed pups, a significantly greater proportion of fetuses from anti-IL1RA pregnancies exhibited less prominent cephalic vasculature, pallid skin color, and the liver appeared pale as viewed from external surface of fetus (FIG. 11), suggestive of a defect in perfusion. Malperfused pups weighed significantly less than normal pups, although defects in gross placental weight were not observed (FIG. 12). Cephalic vasculature was quantified from images of pups, and was significantly lower developed in anti-IL1RA treated pregnancies than isotype controls. Together, these data indicated that anti-IL1RA antibodies were capable of functionally inhibiting IL1RA antagonism of the IL1B pathway and led to resorption as well as vascular defects in placenta and fetus in vivo.

TABLE 1
Top ranked proteins by logistic regression coefficient to classify preterm versus term pregnancies using IP-MS data.

| Protein | Coefficient | Protein | Coefficient | Protein | Coefficient | Protein | Coefficient |
|---|---|---|---|---|---|---|---|
| CCNB2 | 0.107464455 | HDGF | 0.022995045 | THEMIS | 0.006910936 | AMOTL2 | −0.008724711 |
| RPL13 | 0.09211664 | ABCF1 | 0.022853028 | SMC1B | 0.006862376 | SBSN | −0.008805856 |
| ARG1 | 0.07744609 | STOX1 | 0.022849225 | ZP2 | 0.006862376 | AKNAD1 | −0.008819417 |
| PPL | 0.07431845 | HRG | 0.02278947 | ITIH4 | 0.006856106 | CAGE1 | −0.009006679 |
| TEP1 | 0.07369323 | GBE1 | 0.02269088 | MYL4 | 0.006737111 | APP | −0.00901039 |
| ACTB | 0.07286658 | KNG1 | 0.022662211 | TUBB4B | 0.006612103 | SPTB | −0.009066569 |
| TUBA1B | 0.07095573 | HNRNPA1 | 0.022620855 | H1-3 | 0.006606278 | RPL34 | −0.009205502 |
| PRDM12 | 0.06780823 | NUDT4 | 0.022603644 | NSUN2 | 0.006508496 | ORM2 | −0.009391065 |
| FABP5 | 0.06719035 | AHNAK2 | 0.02255229 | DSP | 0.006474346 | CYP2C8 | −0.009528748 |
| DSC3 | 0.06607834 | LCN2 | 0.022507438 | LAMB4 | 0.006361648 | KCNQ4 | −0.009564521 |
| TUBB8B | 0.065959595 | RPL12 | 0.022405114 | FLG2 | 0.006180916 | RAI1 | −0.009606901 |
| C4A | 0.065459095 | RIPK3 | 0.022276139 | GSTT1 | 0.00609554 | TRIM21 | −0.009644072 |
| HNRNPR | 0.06427217 | ARHGEF28 | 0.022260334 | TMEM134 | 0.006063142 | MYH2 | −0.009822072 |
| MPO | 0.063849434 | NSRP1 | 0.02217864 | MNAT1 | 0.006015762 | C4B_2 | −0.009930251 |
| RPS4X | 0.062369455 | CALR | 0.022099549 | IL17RE | 0.005996544 | MESP1 | −0.010093518 |
| PRDX1 | 0.06141654 | CFAP57 | 0.022000883 | MTUS2 | 0.005884389 | FRMPD3 | −0.010180015 |
| H2AC14 | 0.06137082 | HSP90B1 | 0.021945663 | PNP | 0.00584627 | ZNF543 | −0.010193833 |
| RPL27A | 0.061128415 | HBG2 | 0.021924753 | INPPL1 | 0.005844393 | ANXA2P2 | −0.010537746 |
| CBLN2 | 0.060124252 | BCKDHA | 0.021899613 | LARGE1 | 0.005778713 | KLK5 | −0.01055878 |
| POLL | 0.059619013 | ZNF749 | 0.021762293 | CSTB | 0.005760731 | CCDC88A | −0.010707094 |
| TPI1 | 0.05931329 | TEX15 | 0.021710271 | CDSN | 0.005743374 | SSB | −0.01080879 |
| PNISR | 0.059243694 | FNIP2 | 0.021706866 | LGALS3BP | 0.005742781 | ALDH2 | −0.011092434 |
| HSPB1 | 0.05908442 | RPS26P11 | 0.021668043 | LGALSL | 0.005708908 | USP32 | −0.011232066 |
| TUBB8 | 0.059006725 | HTT | 0.021590216 | CLASP1 | 0.005677554 | AKAP14 | −0.011285821 |
| PRDX2 | 0.058492355 | CCNG1 | 0.021560445 | KLF10 | 0.005672907 | PLIN3 | −0.011357769 |
| TUBB4A | 0.057294495 | PUS7 | 0.021483094 | FLNA | 0.005177178 | BORCS7 | −0.011470633 |
| HNRNPCL1 | 0.057083882 | CTSB | 0.021456074 | BANF1 | 0.0051213 | AGT | −0.011480597 |
| SLC14A2 | 0.056630798 | RPS8 | 0.021364002 | CEP192 | 0.00502927 | ITFG1 | −0.011549216 |
| ACTN1 | 0.05615122 | RPS11 | 0.02133116 | SORD | 0.005023851 | C1orf127 | −0.011805891 |
| RAD52 | 0.056109015 | TTC12 | 0.021243323 | GBF1 | 0.004922376 | TNFRSF1A | −0.01180971 |
| S100A9 | 0.05550139 | TRAP1 | 0.02123995 | RPL10 | 0.004916123 | ANKRD12 | −0.011980875 |
| UBA52 | 0.054437548 | HES5 | 0.021185406 | SH3PXD2B | 0.004826464 | DSC1 | −0.01201225 |
| RPS6 | 0.05358796 | H1-5 | 0.021114442 | ABHD18 | 0.004695732 | CNTN3 | −0.012082209 |
| S100A8 | 0.05313724 | GOLGA6L22 | 0.021069372 | ARSB | 0.004412528 | TTN | −0.012203361 |
| HSPA1L | 0.052777123 | RPL24 | 0.02096991 | SYNE1 | 0.004334797 | AIPL1 | −0.012276973 |
| H2BC15 | 0.052708328 | CTSD | 0.020881945 | ZC3H13 | 0.004325999 | ARAP2 | −0.012352079 |
| RPL11 | 0.05264516 | CSN1S1 | 0.020684313 | SHROOM2 | 0.004191038 | HNRNPA2 | −0.012407218 |
| | | | | | | B1 | |
| MYL1 | 0.052594397 | PRSS3 | 0.020670673 | SLC30A8 | 0.003949374 | KIAA0100 | −0.012424602 |
| SPECC1 | 0.051565606 | RPL7 | 0.020646803 | PCM1 | 0.003691886 | LSM14B | −0.012535445 |
| HBB | 0.051464085 | COX7A2 | 0.020619 | WASHC4 | 0.003657959 | SERPINB12 | −0.012616981 |
| CALML3 | 0.051156238 | CLCN5 | 0.020521615 | TENM4 | 0.003615903 | PSD3 | −0.012771297 |
| NEB | 0.050839078 | UTRN | 0.020509489 | C4BPA | 0.003501418 | SERPINF2 | −0.012944069 |
| CWC15 | 0.050455783 | ATP5F1B | 0.02046286 | RPL19 | 0.003484514 | RPL18 | −0.013020821 |
| RBMX2 | 0.050090518 | DOCK5 | 0.020392993 | DNAJC2 | 0.003434202 | ZNF571 | −0.013141119 |
| CALML5 | 0.049874417 | TIGD1 | 0.020388227 | SYNE2 | 0.003340692 | NLGN3 | −0.013345961 |
| HNRNPM | 0.04964356 | TMX2 | 0.020369165 | TBC1D4 | 0.0032081 | IGF2 | −0.013363596 |
| CLUHP3 | 0.049328674 | CSNK1E | 0.02030809 | HMMR | 0.003000258 | SPTA1 | −0.01346179 |
| LGALS7B | 0.049290106 | RPS9 | 0.020188304 | SERPINB4 | 0.002994229 | ZZZ3 | −0.013466439 |
| ZNF236 | 0.04917451 | CTSV | 0.020110698 | AMPD1 | 0.002844173 | LARGE2 | −0.013511526 |
| S100A16 | 0.04910953 | ENO3 | 0.019937158 | RNH1 | 0.002769252 | ELP1 | −0.013592958 |
| H3-3B | 0.049099565 | SLC25A6 | 0.019899083 | BMP1 | 0.002769227 | JAK3 | −0.013593107 |
| SLC25A5 | 0.04894775 | ADAM12 | 0.01979696 | CCDC73 | 0.002769227 | ZMYND10 | −0.013703181 |
| LAMA5 | 0.048823908 | CACNA2D3 | 0.019738294 | ERO1A | 0.002769227 | AHSG | −0.01373523 |
| TCHH | 0.04873567 | CRAMP1 | 0.01958306 | PSMA1 | 0.002715739 | ACIN1 | −0.013810101 |
| CASP14 | 0.04850914 | ACTG2 | 0.019531801 | ZNF837 | 0.002637114 | ZNF665 | −0.013864038 |
| HRNR | 0.048245937 | RPL7A | 0.019476807 | CCDC85A | 0.002584212 | TOP3A | −0.013884118 |
| HNRNPC | 0.04809704 | SLC4A1 | 0.019432755 | AMY1B | 0.002569392 | ACTR2 | −0.014094601 |
| FAM185A | 0.047750324 | KLK7 | 0.019312358 | SH3GL3 | 0.002485233 | SLC38A6 | −0.014172999 |
| SRCAP | 0.04740947 | TEX28P2 | 0.019244017 | NLGN1 | 0.002375215 | EPPK1 | −0.014224567 |
| FN1 | 0.04703445 | KY | 0.019156475 | TUBB3 | 0.002358132 | RPL17 | −0.014350111 |
| NOTCH4 | 0.046761516 | PTMA | 0.018768236 | MYO7B | 0.002215376 | MYH11 | −0.014373703 |
| HEATR4 | 0.046313804 | HTRA1 | 0.018707305 | MSN | 0.002129749 | PLCH1 | −0.014774415 |
| MYO1C | 0.046138946 | NEFH | 0.01861467 | PGK1 | 0.002069049 | CDC5L | −0.014853098 |
| MYH10 | 0.045947533 | MYL12B | 0.0186145 | SYNCRIP | 0.001996521 | PRDM1 | −0.015258748 |
| CFAP99 | 0.045842238 | C18orf63 | 0.018531581 | FAM13A | 0.001930651 | CARMIL1 | −0.015281595 |
| AKT3 | 0.045811724 | GOLGA6L6 | 0.018506605 | TAFAZZIN | 0.001916214 | WDR1 | −0.01549055 |
| KCTD1 | 0.045612622 | ACTBL2 | 0.018197663 | VTN | 0.001714644 | MR1 | −0.015552586 |
| TPM4 | 0.04540783 | HNRNPL | 0.017980997 | EEF1A1 | 0.001679737 | ATP1A4 | −0.015587427 |
| MYL12A | 0.04531687 | POTEKP | 0.017727092 | ZNF624 | 0.00163311 | AKAP9 | −0.015680296 |
| FILIP1 | 0.045306798 | CC2D2B | 0.017405838 | NR3C2 | 0.001554415 | CST1 | −0.015700694 |
| ACTA1 | 0.04505589 | HSP90AB1 | 0.017149676 | CRABP2 | 0.001366889 | SRRM1 | −0.015732339 |
| TPM1 | 0.04481243 | PHETA2 | 0.0169821 | CEBPD | 0.001361926 | ANK1 | −0.015774705 |
| AKT1 | 0.044767268 | ZBED1 | 0.016949967 | MYH1 | 0.00128947 | TREX2 | −0.015787415 |
| RPL3 | 0.044725794 | DHX40 | 0.016892258 | PLS3 | 0.001274546 | DNAH14 | −0.015857477 |
| MYH7B | 0.044386074 | IQCF1 | 0.016830103 | ABCA12 | 0.001122161 | AHNAK | −0.015933746 |
| ANXA2 | 0.044259418 | SPEF2 | 0.016798696 | TXNRD1 | 0.000958903 | SMARCAL1 | −0.015955735 |
| DNMBP | 0.04406121 | GMNC | 0.01655349 | ARHGEF2 | 0.000826203 | ZNF551 | −0.016082104 |
| MGAM2 | 0.043871514 | PIP | 0.016512541 | CRB1 | 0.000783595 | PSME2 | −0.016261602 |
| NCL | 0.043783545 | CDK11B | 0.01642485 | TMEM263 | 0.000715648 | HNRNPH2 | −0.01646747 |
| TF | 0.043672126 | PIK3CD- | 0.016342672 | FAM32A | 0.000686669 | LAP3 | −0.016475756 |
| | | AS1 | | | | | |
| EXOC4 | 0.043653555 | NPM1 | 0.016194202 | XIRP1 | 0.000686669 | BCL9 | −0.016564399 |
| HECW1 | 0.04356021 | MACF1 | 0.01619318 | RPL27 | 0.00050125 | ATPAF1 | −0.016804365 |
| HSPA9 | 0.04347392 | XPO4 | 0.016138356 | MSH3 | 0.000373366 | PFN1 | −0.01714047 |
| GIMD1 | 0.043448616 | ATP8A2 | 0.016136618 | ZNF623 | 0.000359785 | S100A2 | −0.017148739 |
| TYW1B | 0.043370053 | ATP5F1A | 0.016061004 | SHROOM3 | 6.52E−05 | TUBB | −0.01736597 |
| NR1H2 | 0.043366082 | TUBA4A | 0.016017532 | ACP3 | 0 | C1QB | −0.017479956 |
| CHAMP1 | 0.04314315 | CCDC124 | 0.015994849 | AGAP2 | 0 | CRMP1 | −0.01748135 |
| BLMH | 0.04312265 | CACNA2D2 | 0.01594311 | ANKRD54 | 0 | C3 | −0.01756159 |
| EFCAB5 | 0.04304375 | CDAN1 | 0.015894687 | ARHGDIA | 0 | HRH2 | −0.017624628 |
| VCP | 0.043006856 | RBM28 | 0.015894687 | ARPC2 | 0 | ITIH2 | −0.017800234 |
| UBC | 0.042900894 | SLCO2B1 | 0.015894687 | ATP8B3 | 0 | HPR | −0.018215952 |
| NEDD1 | 0.042837866 | ZFC3H1 | 0.015894687 | BASP1 | 0 | GALNT4 | −0.018424688 |
| SLK | 0.042830087 | MTRES1 | 0.015894571 | BRD1 | 0 | PRPF19 | −0.018570168 |
| RPLP2 | 0.042450856 | SPATA5 | 0.015894571 | C1orf54 | 0 | CLTC | −0.019292686 |
| MACROH | 0.04237965 | RPL28 | 0.01576475 | C1QTNF3 | 0 | EEF1B2 | −0.01944882 |
| 2A2 | | | | | | | |
| GGCT | 0.041972626 | RAN | 0.015714148 | CANX | 0 | RTCB | −0.01948807 |
| DGKK | 0.04162661 | SRRM4 | 0.015575511 | CDK5R2 | 0 | C17orf50 | −0.020046778 |
| SRSF2 | 0.04155951 | HBA2 | 0.015514692 | CHGB | 0 | ZFP42 | −0.020131683 |
| EIF3J | 0.041518223 | POLB | 0.015423673 | CHSY3 | 0 | SHANK1 | −0.02022532 |
| H2BC12L | 0.041505642 | RPS3 | 0.015410719 | CLIC2 | 0 | RYR3 | −0.020456312 |
| CKAP4 | 0.041151114 | CST6 | 0.015400851 | CNGA2 | 0 | TPM3 | −0.020700391 |
| MAB21L4 | 0.04095723 | DPYS | 0.01539986 | CPSF6 | 0 | C1S | −0.020720052 |
| NPC2 | 0.040610753 | FNIP1 | 0.015344701 | CRIP2 | 0 | FGG | −0.020954996 |
| SCLT1 | 0.04004154 | SAMD4A | 0.015336132 | DDX3Y | 0 | MUCL1 | −0.021094345 |
| AMBP | 0.040036846 | RPL30 | 0.015311059 | DDX42 | 0 | AKT2 | −0.02136763 |
| TMEM242 | 0.03970524 | PCNX2 | 0.015269655 | DNAJC16 | 0 | SPTBN4 | −0.021738183 |
| REPS2 | 0.0395356 | H2BC11 | 0.015048659 | DOC2A | 0 | GSTM5 | −0.021794874 |
| TEX9 | 0.039520595 | OR2T3 | 0.014971522 | DPYSL2 | 0 | RBBP8 | −0.021918667 |
| CFH | 0.03948112 | KIAA0753 | 0.014971462 | EEF1G | 0 | SEPSECS | −0.022179523 |
| AZGP1 | 0.0393481 | HNRNPA1L2 | 0.014958161 | FANCB | 0 | MOV10L1 | −0.022334218 |
| RPLP1 | 0.038884178 | PSMB3 | 0.014941451 | GALNTL6 | 0 | MYH9 | −0.022728473 |
| CCDC168 | 0.038522866 | MMP16 | 0.014779775 | GIGYF2 | 0 | FBXO10 | −0.023009004 |
| TTC41P | 0.038460407 | MAPKAPK5 | 0.014530829 | GMEB2 | 0 | MYL6B | −0.023328783 |
| ORC1 | 0.037939668 | GNMT | 0.014453423 | HSD17B2 | 0 | RPL4 | −0.023780223 |
| PRSS1 | 0.03786236 | ANK3 | 0.014352912 | ITGA7 | 0 | CUL3 | −0.023848414 |
| HSP90AA1 | 0.037855238 | HNRNPA3 | 0.014350839 | KIAA1143 | 0 | VPS35L | −0.023892151 |
| DDN | 0.037582 | PCNT | 0.014312659 | KIF16B | 0 | EVPL | −0.023992186 |
| RPS14 | 0.037533693 | ATRX | 0.014268402 | LUZP1 | 0 | PKP3 | −0.024041696 |
| H2AX | 0.037486497 | MYL9 | 0.014241285 | LYRM7 | 0 | DACH1 | −0.024054818 |
| ASXL2 | 0.037415676 | SARAF | 0.014060739 | MARCHF3 | 0 | ABCA10 | −0.024069931 |
| HYLS1 | 0.0373357 | ANXA1 | 0.013976894 | MDH1 | 0 | CRYAB | −0.024219612 |
| C22orf23 | 0.037313525 | TUBB2B | 0.01397375 | NAV1 | 0 | GSTP1 | −0.02436466 |
| C1RL | 0.036332704 | C2CD3 | 0.013755651 | NRG2 | 0 | MAOB | −0.024475219 |
| MIPOL1 | 0.035959333 | ENO1 | 0.013692553 | PAAT | 0 | VIM | −0.024478251 |
| TSPY10 | 0.035959333 | SMAP2 | 0.013689061 | PCBP1 | 0 | GRIPAP1 | −0.024480654 |
| HEATR5A | 0.035877172 | CCNI | 0.013640595 | PEX14 | 0 | MAGEH1 | −0.024552675 |
| CPA4 | 0.035844594 | BFSP2 | 0.01356418 | PHB2 | 0 | SERPINA1 | −0.024610158 |
| PCLO | 0.035733934 | GHRL | 0.01353305 | PHF21A | 0 | ERC1 | −0.024975473 |
| RBPJ | 0.03565744 | SRSF3 | 0.013511292 | PIWIL2 | 0 | TGM2 | −0.025074437 |
| H1-4 | 0.035474375 | TUBA8 | 0.013509616 | POLI | 0 | EIF4A3 | −0.025202082 |
| PKM | 0.035464108 | PSMB4 | 0.013486272 | RAI14 | 0 | ARF1 | −0.02523533 |
| GAPDH | 0.035452984 | KMT2C | 0.013417752 | RBAK | 0 | ALDH1A3 | −0.025476027 |
| CEP97 | 0.035249628 | NUDT18 | 0.013417752 | RDX | 0 | CTPS1 | −0.026246995 |
| VARS2 | 0.03506995 | PSMA8 | 0.013417752 | RNASE7 | 0 | FSCN1 | −0.026538514 |
| SOD1 | 0.034631584 | H1-2 | 0.013316516 | SERPINB13 | 0 | TOLLIP | −0.026873978 |
| P4HB | 0.034237035 | FCGBP | 0.013313946 | SLC2A6 | 0 | NIN | −0.026905373 |
| CAST | 0.034200698 | AJM1 | 0.01327512 | SMIM21 | 0 | TFPI2 | −0.026914712 |
| TRPM2 | 0.034161493 | ANKRD18B | 0.013087755 | SNX11 | 0 | RABEP1 | −0.026940908 |
| KIF12 | 0.034149498 | CFL1 | 0.013087356 | SSR4 | 0 | LRRC27 | −0.027009502 |
| TRIM29 | 0.034033705 | MYCNOS | 0.012913294 | STAT1 | 0 | TECTA | −0.02714235 |
| RPL32 | 0.033930134 | PKP1 | 0.012910703 | TICRR | 0 | GPI | −0.027226187 |
| CLK1 | 0.03392409 | F2 | 0.012899789 | TNXB | 0 | GPR39 | −0.027275482 |
| MYH3 | 0.033722132 | PGK2 | 0.012865227 | TPT1 | 0 | RAB14 | −0.027365923 |
| HAUS8 | 0.03365661 | SLC25A4 | 0.012849396 | WASL | 0 | OR6T1 | −0.027719095 |
| DNAJC13 | 0.033602163 | ATAD2 | 0.012770486 | ZFYVE27 | 0 | STT3B | −0.027761275 |
| TGM3 | 0.033393037 | LAMA3 | 0.012733022 | ZNF331 | 0 | PLEC | −0.027959943 |
| CSTA | 0.033214435 | ASPM | 0.012716194 | MYH4 | −1.08E−05 | EIF3H | −0.02796496 |
| PHLPP1 | 0.032775085 | SAMD15 | 0.012658449 | SAMM50 | −2.48E−05 | ZNF594 | −0.02796496 |
| POF1B | 0.03269619 | NBDY | 0.012475222 | NBAS | −0.000137224 | IRAK4 | −0.028109394 |
| TIAM2 | 0.032682117 | CDIN1 | 0.012299993 | GOT1 | −0.000225634 | CARD11 | −0.028117258 |
| S100A7 | 0.032495774 | HNRNPU | 0.012275902 | KLK10 | −0.000225634 | ZNF33A | −0.028374868 |
| MYH14 | 0.032430455 | RPS27A | 0.012265593 | MPV17L | −0.000225634 | SPRR2B | −0.028814524 |
| IL1RN | 0.0321926 | JUP | 0.0121053 | THADA | −0.000225642 | S100A14 | −0.029063947 |
| ERC2 | 0.03204188 | CDK13 | 0.012090708 | TAGLN2 | −0.000347834 | SERPINA12 | −0.029137693 |
| YWHAZ | 0.031702165 | KIF2C | 0.012045796 | RPL6 | −0.000392273 | RUNX1T1 | −0.029281318 |
| H3-7 | 0.031122783 | VDAC2 | 0.012034528 | CEP83 | −0.000398387 | DISP3 | −0.029313039 |
| PZP | 0.031068483 | CPXM1 | 0.012006458 | ATG16L2 | −0.000410813 | RAB7A | −0.029390471 |
| ALDOA | 0.0308783 | ZNF318 | 0.011693067 | A2ML1 | −0.000720994 | HAUS7 | −0.029471682 |
| GOLGA3 | 0.030858377 | TYMP | 0.011689949 | HESX1 | −0.000790203 | RET | −0.029471682 |
| ECM1 | 0.030742435 | EIF4A1 | 0.011633016 | ABCC2 | −0.001073947 | MYH13 | −0.029502977 |
| STK25 | 0.03062224 | CDKL5 | 0.011517547 | MYO1D | −0.001107642 | ABCA1 | −0.029874396 |
| RPL8 | 0.030578613 | HK1 | 0.011508668 | MGST3 | −0.001194372 | ACSM3 | −0.029972978 |
| LYZ | 0.030385526 | RBPMS | 0.011429595 | THSD4 | −0.00120304 | PRSS3P2 | −0.030156532 |
| ACTA2 | 0.03031138 | PURB | 0.01126193 | H4-16 | −0.001222658 | FSIP2 | −0.030221269 |
| S100A11 | 0.03016853 | CHST10 | 0.01119055 | CSN2 | −0.001396025 | ZNF180 | −0.030490825 |
| HNRNPK | 0.030044658 | TXN | 0.011119092 | ZSCAN22 | −0.001471197 | ZNF33B | −0.030674066 |
| TPM2 | 0.030001732 | MRTO4 | 0.011108389 | NALCN | −0.00148613 | PIK3CA | −0.031031722 |
| DBT | 0.029909424 | C8orf34 | 0.01095272 | LRRD1 | −0.00165325 | PTGES2 | −0.031031722 |
| RO60 | 0.029605053 | TTLL4 | 0.0109353 | MTMR3 | −0.001664384 | SLURP1 | −0.031031722 |
| SSBP1 | 0.029582039 | ALB | 0.010904592 | NID1 | −0.001741549 | SNRPD2 | −0.031031722 |
| LRRC74B | 0.029308097 | GTF2F2 | 0.010872591 | CFAP44 | −0.001771485 | DNAJC21 | −0.031031774 |
| FLG | 0.029094882 | SERPINB3 | 0.010809678 | MS4A1 | −0.001893076 | SYTL4 | −0.031031774 |
| RBMS2 | 0.028681256 | HBD | 0.010785114 | YWHAQ | −0.001983714 | ZNF596 | −0.031031774 |
| HS6ST2 | 0.028537072 | ACTC1 | 0.010782463 | CLIC3 | −0.002042455 | CTTNBP2 | −0.031245708 |
| SNRNP200 | 0.02851049 | MYO1B | 0.010746676 | EEA1 | −0.00209053 | ASB7 | −0.031304676 |
| PLXNA1 | 0.02846261 | ADGRD2 | 0.010624478 | ZBTB40 | −0.002159125 | CLTCL1 | −0.03172868 |
| REEP2 | 0.028462606 | APOD | 0.010516868 | PADI2 | −0.002194976 | FILIP1L | −0.031758085 |
| S100P | 0.028369954 | GCN1 | 0.010464093 | APOA1 | −0.002351968 | ZNF721 | −0.032078963 |
| RPL18A | 0.028330898 | PRKDC | 0.010464093 | NEK5 | −0.002817275 | GSN | −0.032127447 |
| CABP7 | 0.028277414 | RPL37A | 0.010464093 | RIBC1 | −0.002902494 | CEP250 | −0.03351095 |
| GTF3C4 | 0.028254736 | ZNFX1 | 0.010464093 | UST | −0.002998301 | DNMT1 | −0.03431671 |
| TUBB6 | 0.02816615 | C8G | 0.010464021 | SPAG5 | −0.003029623 | TMOD3 | −0.034374233 |
| HSPA2 | 0.028145451 | CST4 | 0.010464021 | VPS13C | −0.003142401 | XRCC6 | −0.034446023 |
| HSPA5 | 0.028058408 | EFR3B | 0.010464021 | XRCC1 | −0.003166874 | NT5C3A | −0.034556963 |
| RPS2 | 0.028020157 | RPL29 | 0.010128944 | HSPA1A | −0.003207492 | SPDL1 | −0.034556963 |
| PALM2 | 0.027959507 | SLC22A8 | 0.010121495 | TTC21A | −0.003234108 | GRAMD1B | −0.034702927 |
| CHD7 | 0.027848423 | ANKRD2 | 0.01011503 | DNAJC24 | −0.003422117 | TCFL5 | −0.03473379 |
| | | 0A8P | | | | | |
| HNRNPH1 | 0.027535228 | ZFP82 | 0.010004902 | MUC16 | −0.003489811 | SERPINB1 | −0.03480488 |
| SPTBN2 | 0.027374959 | YBX1 | 0.009925976 | MAGED1 | −0.003559792 | MYL3 | −0.0355238 |
| RPL23A | 0.027317006 | NEXN | 0.009735917 | POTEI | −0.003632187 | EXOC1 | −0.035755068 |
| SUB1 | 0.027314914 | CLU | 0.009692624 | YWHAE | −0.003690512 | REST | −0.035811245 |
| ACSM5 | 0.027262086 | CCDC43 | 0.009636742 | RAB11FIP1 | −0.003860893 | PDE4DIP | −0.036017165 |
| DNAH12 | 0.027191065 | NOC3L | 0.009576451 | C2orf16 | −0.003891489 | DMD | −0.036324114 |
| HSPA8 | 0.027045531 | NUCB1 | 0.00957115 | PLEKHA6 | −0.004335695 | H3C13 | −0.036356196 |
| COL14A1 | 0.027037866 | YEATS2 | 0.009497103 | WRAP73 | −0.004712132 | SELE | −0.03637002 |
| RACK1 | 0.027020378 | ZNF804A | 0.009496314 | PACSIN1 | −0.004778677 | YWHAG | −0.036458295 |
| PLGLB2 | 0.027014859 | NSD1 | 0.009381964 | C1QC | −0.004840734 | DOCK9 | −0.036809556 |
| CADPS2 | 0.026972322 | DHTKD1 | 0.00937821 | ACTN4 | −0.004848058 | AEBP1 | −0.037335616 |
| FBLN1 | 0.026972322 | PRSS2 | 0.009357695 | TSEN34 | −0.004879362 | A2M | −0.037544176 |
| CACNG3 | 0.02697232 | NPIPA2 | 0.009179902 | SFN | −0.004900939 | ORM1 | −0.037749812 |
| DACT1 | 0.026857384 | GLUL | 0.009152146 | IWS1 | −0.005205117 | CCNB1 | −0.03864533 |
| CTSC | 0.026828066 | PPIA | 0.009148794 | SYNE3 | −0.005269098 | GRN | −0.040381167 |
| CAT | 0.026707852 | ZMYND8 | 0.009038831 | PSIP1 | −0.005507083 | POTEE | −0.04122144 |
| MYT1L | 0.026627209 | MFN2 | 0.009035613 | PSMB6 | −0.005696818 | CD5L | −0.041664265 |
| HNRNPH3 | 0.026256997 | CCDC180 | 0.008827052 | HEXA | −0.005810228 | FOXP1 | −0.041978657 |
| LMNA | 0.026148094 | KIAA1328 | 0.008681732 | PSMA6 | −0.005845867 | KSR2 | −0.04240564 |
| RAD50 | 0.02612918 | FBXW10 | 0.008669676 | TGM1 | −0.005994483 | OXCT2 | −0.04261962 |
| DSG1 | 0.026118413 | PAK6 | 0.00858862 | PPOX | −0.006077438 | MYL6 | −0.042718522 |
| SPATA7 | 0.026066687 | RPL15 | 0.008345618 | EZR | −0.006227023 | CCDC88B | −0.04370107 |
| CALM1 | 0.025996933 | MOV10 | 0.008277481 | PATZ1 | −0.006253305 | ZNF556 | −0.044638425 |
| NBN | 0.025949622 | GRXCR2 | 0.008174774 | CKMT1B | −0.00631927 | ROCK1 | −0.04469759 |
| SENP1 | 0.02583169 | RPL14 | 0.008097695 | FIGN | −0.00631927 | RFX4 | −0.044920664 |
| SRSF11 | 0.025815286 | ARMC2 | 0.007977594 | FUNDC2 | −0.00631927 | ZCWPW1 | −0.044978343 |
| CCDC57 | 0.025334252 | DCD | 0.007976908 | IL36G | −0.00631927 | FGA | −0.045564678 |
| ERICH3 | 0.025243845 | H2BC13 | 0.007946249 | KIF14 | −0.00631927 | ZC3H3 | −0.0457855 |
| NCCRP1 | 0.025233528 | ZNF304 | 0.007946249 | RAB1B | −0.00631927 | MAMSTR | −0.04596955 |
| TAOK2 | 0.02521699 | C2 | 0.007946241 | TEC | −0.00631927 | DHX9 | −0.046029598 |
| CAPZA1 | 0.025172647 | CBR1 | 0.007946241 | TCERG1 | −0.006398206 | VWF | −0.04700197 |
| SIGLEC11 | 0.024949236 | CCDC60 | 0.007946241 | PRKAR2A | −0.006424207 | ATP6V0A2 | −0.047229934 |
| NLRP12 | 0.024851993 | HIF1AN | 0.007946241 | FBL | −0.006432759 | RPL10A | −0.04750332 |
| PDIA3 | 0.024738748 | CP | 0.007900346 | RINT1 | −0.00656245 | SH3BGRL3 | −0.048717465 |
| GOLT1A | 0.024381474 | PTPDC1 | 0.00789984 | MAP1A | −0.006572839 | RBMS1 | −0.04897866 |
| LDHA | 0.024348423 | TTC6 | 0.007880702 | UVSSA | −0.006574593 | UGT2B11 | −0.049454514 |
| DNAJC17 | 0.024288654 | AQR | 0.007661926 | IVL | −0.006694972 | C9orf78 | −0.049936976 |
| ACP4 | 0.024245264 | PSMA5 | 0.007612769 | ILF3 | −0.00690534 | C1QA | −0.050294735 |
| DEFA3 | 0.024206538 | FBXO28 | 0.007603374 | MPP4 | −0.007010531 | KIF25 | −0.051375907 |
| KCNJ13 | 0.023974443 | PLXNB3 | 0.007510269 | PLEKHH1 | −0.007134108 | KIF20B | −0.052072026 |
| HMG20A | 0.02373648 | POTEF | 0.007500786 | CCDC146 | −0.007141858 | TTR | −0.053344723 |
| H2AC20 | 0.02373361 | HPX | 0.007489543 | RPL21 | −0.007220792 | ZSCAN32 | −0.053984098 |
| MTUS1 | 0.023467593 | ANXA8 | 0.007095119 | EFHD2 | −0.008149715 | IDE | −0.054909755 |
| RBM25 | 0.023417117 | MUC19 | 0.007014409 | UTP20 | −0.008417596 | FGB | −0.05590493 |
| P4HTM | 0.023396898 | ST3GAL6 | 0.007004291 | KAAG1 | −0.00850294 | HP | −0.05611526 |
| DPYSL3 | 0.023354532 | SLC22A23 | 0.006964724 | NARS1 | −0.008603285 | MGA | −0.057280574 |
| LMO7 | −0.05737973 | ZNF329 | −0.062119294 | MTMR4 | −0.07068894 | | |
| MRPL10 | −0.061081313 | PDE6C | −0.062347006 | C1R | −0.07084581 | | |

TABLE 2
PhIP-seq preterm-specific peptides validated by IP-MS and transferred from mother
to fetus.

| NCBI protein ID* | Amino acid sequence | SEQ ID NO |
|---|---|---|
| XP_011543551.1 | MPDVELNLKSPKVKGDLDIAGPNLEGDFKGPKVDIKAPEVNLNAPDVDV | SEQ ID NO: 3 |
| NP_776214.1 | CRPSGRKSSKMQAFRIWDVNQKTFYLRNNQLVAGYLQGPNVNLEEKIDV | SEQ ID NO: 4 |
| NP_003557.2 | HRIHVELSEKGEATQKLKEELSEVETKYQHLKAEFKQLQQQREEKEQHG | SEQ ID NO: 5 |
| NP_060670.1 | PPEEKQRYLDEADRDKERYMKELEQYQKTEAYKVFSRKTQDRQKGKSHR | SEQ ID NO: 6 |
| NP_071900.2 | MLKDMHDSKTKEQRLMTAQNLVSYRSPGRGDCSTNSPVGVSKVLVSGGS | SEQ ID NO: 7 |
| NP_057735.2 | RTDYRRWKSSAEISYEEALRRARRGRRENVGLYPAPVPLPYASPYAYVA | SEQ ID NO: 8 |
| NP_001161206.1 | TAQVYMELQGLVDPQIQLPLLAARRYKLQKQLDSLTARTPSEGEAGTQR | SEQ ID NO: 9 |
| NP_001900.1 | PGGELMLGGTDSKYYKGSLSYLNVTRKAYWQVHLDQVEVASGLTLCKEG | SEQ ID NO: 10 |
| XP_005273028.1 | LKGQVETIVSFHIQNISNNKTERSTAPLNTQISALRNDPKPLPQQPPAP | SEQ ID NO: 11 |
| XP_005263216.1 | VKAAEGPVLEPVAPRCVQRGPGLVAGANIPCGDDNKKANFRTEGDIPRN | SEQ ID NO: 12 |
| XP_005266221.1 | AELQAVSHSKTLLEKELQEVIALTSQELEESREKVLELEDELQESRGFR | SEQ ID NO: 13 |
| XP_006713806.1 | LQVWQQTVDQRLSSWHTDVPPISSTLVPSKHPLFTQSQESSCDQNADWF | SEQ ID NO: 14 |
| NP_001013764.1 | RRSRRGTLSSISSLKSEDLDNLNLDHLHFTPESIRFKEKCVMNNYFGIG | SEQ ID NO: 15 |
| XP_011532914.1 | LESNDLDPGFMPKKGDLGLSKKCYEAGHLENGITESCATSYSKDFGGGT | SEQ ID NO: 16 |
| XP_011514753.1 | NCFILYSSTAQAKNSENKESIPSLPQSPMRETPSKAFHQYSNNISTLDV | SEQ ID NO: 17 |
| XP_011537116.1 | EMEKEIVSSTRLDLQKKSEALESIKQKLTKQEEEKKILKQDFETLSQET | SEQ ID NO: 18 |
| XP_011523977.1 | NANRGGFDLTDPVKQGAECPHQNKTVLHMDGCLDTETPTVSIQENVDVA | SEQ ID NO: 19 |
| NP_149015.2 | DRSGPYGYRGIGGMKPSMSDTNLAEAGHFFYKSKNAFDYSEGTDTAVDL | SEQ ID NO: 20 |
| NP_001123476.1 | ETIDQLYLEYAKRAAPFNNWMEGAMEDLQDTFIVHTIEEIQGLTTAHEQ | SEQ ID NO: 21 |
| NP_054733.2 | SANLVASGELMSSKKKDLHPRDIDAFWLQRQLSRFYDDAIVSQKKADEV | SEQ ID NO: 22 |
| XP_005264966.1 | IQGGNLLEEQPMSPSGNRMQESQETAAEGTLRTLHATPGILHHGGILME | SEQ ID NO: 23 |
| XP_005263213.1 | CEVLGAGMKMDQQAVCELLKVEMPTRLPDRSVAWPCPDRHLREKPSLEK | SEQ ID NO: 24 |
| NP_006120.1 | RYCGSGPPEEVYSAGDSVLVKFHSDDTITKKGFHLRYTSTKFQDTLHSR | SEQ ID NO: 25 |
| XP_006712126.1 | PAAVVERNTLMVIKCLMENLEEEVSQAENSLLQAAAAFPMYGRVHCITG | SEQ ID NO: 26 |
| XP_011514754.1 | APTVEGENDTMSNAQRSTLKWEKEEALGEMATVAPVLYTNINFPNLKEE | SEQ ID NO: 27 |
| XP_011543226.1 | NLVEWRQRKEQARGQVTVFGRRLRVHNRNLLSLDFDRVTRTEKIYDDHR | SEQ ID NO: 28 |
| XP_011543551.1 | FKGEGPEVDVNLPKADLDVSGPKVDVDVPDVNIEGPDAKLKGPKFKMPE | SEQ ID NO: 29 |
| XP_011543551.1 | KMQVGGDGVKVPGIDATTKLNVGAPDVTLRGPSLQGDLAVSGDIKCPKV | SEQ ID NO: 30 |
| XP_011528038.1 | TDNAWIETVAVSVHFQDQNDVELNRLNSNLHACDSGASIRWQVVDRRIP | SEQ ID NO: 31 |
| NP_001074248.1 | LLNLRRQLPQNVLDTSWPTPPPALREASELLRELCIKNMVWKYCRSISP | SEQ ID NO: 32 |
| NP_149015.2 | RRLTRKSSTSIDEDAGRRHSWHDEDDEAFDESPELKYRETKSQESEELV | SEQ ID NO: 33 |
| NP_060547.2 | CPKKEPKAKKAKVADKKARLIIRNLSFKCSEDDLKTVFAQFGAVLEVNI | SEQ ID NO: 34 |
| NP_001123633.1 | LGKQKVIERSLTEYNSTMERVESQVLKRNECYKPTGLHATPGESQEPNL | SEQ ID NO: 35 |
| NP_659451.1 | PQSKPQHIYVPNNYLVPTEKKRSALRWGVRCDLANGVIPRKLPFPLSPS | SEQ ID NO: 36 |
| NP_001139281.1 | QRMNPPKFSKAEDMAELTCLNEASVLHNLRERYYSGLIYTYSGLFCVVI | SEQ ID NO: 37 |
| NP_001156729.1 | GTPFSQRPSVHALDLEWRSGLAGHLTLSDEDLTSVTQNHWKRLNTLQHY | SEQ ID NO: 38 |
| NP_001161206.1 | ALGASLDWDRECFTMDVGSSVAVTEAFVRLYKAGLLYRNHQLVNWSCAL | SEQ ID NO: 39 |
| NP_733822.1 | THSPPTDLVWKAQNTWGCGNSLRTALINSTGEGSHCSSSGDPAEYNLRS | SEQ ID NO: 40 |
| NP_005886.2 | QLEQVKLTLLQRDQQLEALQQEHLDLMKQLTLTQEALQSREQSLDALQT | SEQ ID NO: 41 |
| NP_056097.1 | RKGALLELLKITREDSLGVWEEHFKTILLLLLETLGDKDHSIRALALRV | SEQ ID NO: 42 |
| NP_919226.1 | LLDSHQLLDKRPKSESISLSDNEEMCKTWNTEYNTYDTISSKNHCKKNT | SEQ ID NO: 43 |
| NP_001001931.1 | RAHVHLMKTPPKGPSRKNLFTALNAVEKSRQKNPRSLCIQPQTAPDALP | SEQ ID NO: 44 |
| XP_005262738.1 | SSTKLSELHDNQDGLVNMESLNSTRSHERTGPDDFEWMSDERKGNEKDG | SEQ ID NO: 45 |
| XP_005263216.1 | GSFASPESDFESRMKKMEERVKACGPSLEASEAADVAQDPQVSRSPFKP | SEQ ID NO: 46 |
| XP_005250082.1 | EPETVVSREESRPPKLVMESVTLPLETLVSPHEESISLCPEEQLVIERL | SEQ ID NO: 47 |
| XP_005268896.1 | NVKDKLRAIVVTLSYSLQTPRLRRQAPGQGLPPVAPILNAHQPSTQRAE | SEQ ID NO: 48 |
| XP_005266221.1 | AKSGKEELDRGARRLEEGTEETSETLEKLREELAIKSGQVEHLQQETAA | SEQ ID NO: 49 |
| XP_005257841.1 | LVTSIAKACALDPGTRATGGSLSTGNNDTSEACDADFDSSLTACSGVES | SEQ ID NO: 50 |
| XP_005258164.1 | HFLAKVASSRKQHLPLKNAGNIEVYLDIKVPEQGSHFSVDPKNLLLKPG | SEQ ID NO: 51 |
| NP_775871.2 | WAVRKCEILRDSTFRDCHNKVDPSAYHDACIEEACACDMEGKYLGFCTA | SEQ ID NO: 52 |
| NP_066933.1 | DETTSSLRVKWDISDSDVQQFRVTYMTAQGDPEEEVIGTVMVPGSQNNL | SEQ ID NO: 53 |
| XP_006726566.2 | RRAGARGLGAGHPVPSGGILVLGQDQDSLGGGFSVRDAFSGNLTDFHLW | SEQ ID NO: 54 |
| XP_006726566.2 | PQPFLCCYRTEPYRRLQDAQSWPGQDVISRVNALANDIVLLPDPLSEVH | SEQ ID NO: 55 |
| XP_011531004.1 | TKIRMSGIKGLQGVVRKTVNDELQANIWDPQHMDKIVPSLLFNLQHVEE | SEQ ID NO: 56 |
| XP_011514754.1 | TSDHFTKPSPRADVFQRQRIPDSYARPLLTPAPLDSGPGPFKTPMQPPP | SEQ ID NO: 57 |
| XP_011515869.1 | ELPAKRVRGKARLPPDVLRWVELAKLYRSIGEYDVLRGIFTSEIGTKQI | SEQ ID NO: 58 |
| XP_011543231.1 | AGLLIKAYNRAGSWSVRYRYDGLGRRVSSKSSHSHHLQFFYADLTNPTK | SEQ ID NO: 59 |
| XP_011523974.1 | PDRQLDVTARGVYAPEDVYRFRPTSVGESRTLKVNLRNNSFITHSLKFL | SEQ ID NO: 60 |
| XP_011525784.1 | DSCGLTAKACENLYFTLGINQTLTDLYLTNNALGDTGVRLLCKRLSHPG | SEQ ID NO: 61 |
| NP_060338.3 | TASRYAIKILAICTNSYHEAREEVIRLDKKLSVMMKLLSSEDEVLVGNA | SEQ ID NO: 62 |
| NP_001074248.1 | SKNPIMSQCFDRSELSDKKRPETVATQFKMSLLQLVEILQSKEPAYVRC | SEQ ID NO: 63 |
| NP_001932.2 | VCSANGFMTQTTNNSSQGFCGTMGSGMKNGGQETIEMMKGGNQTLESCR | SEQ ID NO: 64 |
| NP_001095886.1 | FSPFRSSRIGEIKEETTVSPPHTSMAPAQDEERDSGKEQGHTRRHDWGH | SEQ ID NO: 65 |
| NP_612429.2 | RLEGDLSLAHEDVAGKDSKFQGPKLSTSGFEWSSKKVSMSSSEIEGNVT | SEQ ID NO: 66 |
| NP_006175.2 | SLGEEQRKEAERKLEEQQRRHREHPKVNVPGSQAQLKEVWEELDGLDPN | SEQ ID NO: 67 |
| NP_001158887.1 | HGDSSVPVWSGMNVAGVSLKTLHPDLGTDKDKEQWKECRYTLGDPKGAA | SEQ ID NO: 68 |
| NP_001161206.1 | EAFVRLYKAGLLYRNHQLVNWSCALRSAISDIEVENRPLPGHTQLRLPG | SEQ ID NO: 69 |
| NP_001002912.4 | DEEKQGEKSNEEGQADVQMNGIPQSPLDDKKDNLDPEKESETSSQKAPD | SEQ ID NO: 70 |
| NP_005886.2 | RLQEALAAKEAADAELGQLRAQGGSSDSSLALHERIQALEAELQAVSHS | SEQ ID NO: 71 |
| NP_001182225.1 | ALIEERDAALSKCKRLEQELHHVKEQNQTSANNMRHLTAENNQERALKA | SEQ ID NO: 72 |
| NP_078824.2 | WHTDVPPISSTLVPSKHPLFTQSQESSCDQNADWFIASDVAPQEKSLPE | SEQ ID NO: 73 |
| NP_001258908.1 | TFVGTPFWMAPEVIKQSAYDFKADIWSLGITAIELAKGEPPNSDLHPMR | SEQ ID NO: 74 |
| NP_000403.1 | CEVFDPQEHENINGVPPHLGHPFHWGGHERSSTTKPPFKPHGSRDHHHP | SEQ ID NO: 75 |
| XP_005273028.1 | HGPRGVVRGPPPPYQMTPSEGWAPGGTEPFSDGINMPHSLPPRGMAPHP | SEQ ID NO: 76 |
| XP_005264966.1 | IRKEELVSGELPRIICQVLRRPDVDQQGLLVQEDPTGQLQLKPLRLPTP | SEQ ID NO: 77 |
| XP_005248225.1 | HNPISLAMTLKTLDEHRDKAVTQLGSMLFTRQVSGARVVPLGSMQTVSG | SEQ ID NO: 78 |
| XP_005262738.1 | GGHTQHFESPTMKIQEHPSLSDTKQQRNQDAGDQEESFVSEVPQSDLTA | SEQ ID NO: 79 |
| XP_005250082.1 | SEDDCTKDNKLVEKQNPAEGLQTLGAQMQGGFGCGNQLPKTDGGSETKK | SEQ ID NO: 80 |
| XP_005250082.1 | PCASLSSCPVCYRNYREEDLILQCRQCDRWMHAVCQNLNTEEEVENVAD | SEQ ID NO: 81 |
| XP_005250088.1 | NPPTPPASLPPTPPPMACQKMANGFATTEELAGKAGVLVSHEVTKTLGP | SEQ ID NO: 82 |
| XP_005259118.1 | GVLVDQSWVLTAAHCGNKPLWARVGDDHLLLLQGEQLRRTTRSVVHPKY | SEQ ID NO: 83 |
| NP_000480.3 | FIISTKAGSLGINLVAANRVIIFDASWNPSYDIQSIFRVYRFGQTKPVY | SEQ ID NO: 84 |
| NP_775871.2 | VTSGQSARVTGATGPSAGETGTTEPSTEGSVAAVLFVIGSETTRPLDIG | SEQ ID NO: 85 |
| NP_775871.2 | AVVTGINGLSAGVTGTTGPSAEETGATGPSAEVTGTTGPSGGVTGTSGI | SEQ ID NO: 86 |
| NP_775871.2 | SRRGILTLMWDKNNKLSLTLHKQYPTCGLCGNFNSTPGQDINEHIANSK | SEQ ID NO: 87 |
| NP_775871.2 | SGALGSTEGSVEATTSAGSGNTAGTSGTGDTGPGNTAVSGTPVVSPGAT | SEQ ID NO: 88 |
| NP_003557.2 | ELNSQLNESKEKVSQLDIQIKAKTELLLSAEAAKTAQRADLQNHLDTAQ | SEQ ID NO: 89 |
| NP_003557.2 | INELKQSSEQKKKQIEALQGELKIAVLQKTELENKLQQQLTQAAQELAA | SEQ ID NO: 90 |
| XP_006710413.1 | EGQGGVDVVLRTQEAVAEEDPIMAEKFREEAVDEDPEEEEDKECTLETE | SEQ ID NO: 91 |
| XP_006710413.1 | EEREVLAGSETAEEKTIANKASSFSDVAEEETWHQQDELVGKTAAAGKV | SEQ ID NO: 92 |
| XP_006712124.1 | TTAMSQENTCQSTEFAFCQVDASIALALALAVLCDLLQQWDQLAPGLPI | SEQ ID NO: 93 |
| XP_006712126.1 | ALGALMACLRIARAHGHLQSATDTWENLVSDARIKQGLIHQHCQVRIDT | SEQ ID NO: 94 |
| XP_006718110.1 | SEVMIHMGLHLLTVALESAPVAQCQTLLGLIKDEMCRHLFQLLSIERLN | SEQ ID NO: 95 |
| XP_006718110.1 | HTLHTRAASIYSSWAEEQRHLETGGQKIEADSRTLWAHCWCPLLQGIAC | SEQ ID NO: 96 |
| XP_006718732.1 | TKWQKHQAFMAELAANKDWLDKVDKEGRELTLEKPELKALVSEKLRDLH | SEQ ID NO: 97 |
| XP_006718732.1 | AQLQGSSAARRQLLLDTTDKFRFFKAVRELMLWMDEVNLQMDAQERPRD | SEQ ID NO: 98 |
| XP_006723899.1 | DFCCQEPCSKSLRCGHRCSHPCGEDCVQLCSEMVTIKLKCGHSQPVKCG | SEQ ID NO: 99 |
| NP_009187.1 | PGLELSPSCMEACSCPLDGFNPVCDPSTRVEYITPCHAGCSSWVVQDAL | SEQ ID NO: 100 |
| NP_001020268.1 | IVGFDDDGISRLYQTDPSGTYHAWKANAIGRSAKTVREFLEKNYTEDAI | SEQ ID NO: 101 |
| NP_053585.1 | QPSYRWFKDGKELNRSRDIRIKYGNGRKNSRLQFNKVKVEDAGEYVCEA | SEQ ID NO: 102 |
| NP_053586.1 | HHCSTATPTSSHRHESHTWSLERSESLTSDSQSGIMLSSVGTSKCNSPA | SEQ ID NO: 103 |
| XP_011508537.1 | SSLNALTHSSRLKLHLKSDMSECENDDPLLRSADEDPQVIEMMADKKYK | SEQ ID NO: 104 |
| XP_011509972.1 | RGSGKSNVGTSGDHDDSAMKTLRSKMGKWCCHCFPCCRGSGKSNVGAWG | SEQ ID NO: 105 |
| XP_011512149.1 | FTRQVSGARVVPLGSMQTVSGYTFRGFMSHINNYPCAYLNAASAIGMKM | SEQ ID NO: 106 |
| XP_011529819.1 | FSSWQRENSDSDEAHLSPQAGRLIRQLLDEDSDPMLSPRFYAYGQSRQY | SEQ ID NO: 107 |
| XP_011512437.1 | GRGKSDTSQKKPKELLESLLQADIVKFAHFLLDVMNVLSILSCVNKNRN | SEQ ID NO: 108 |
| XP_011512442.1 | RLAEKSLPPRAESTTPELPSFAVKGCLLGKTLSGKTTILRSLQKDFPIQ | SEQ ID NO: 109 |
| XP_011512442.1 | LLYFACHPDTVEGVYRALSVAVGTHVFQQVKASIPSAEKTSSTDAGPAE | SEQ ID NO: 110 |
| XP_011532914.1 | SEKKRLRKPSKWLLEYTEEYDQIFAPKKKQKKVQEQVHKVSSRCEEESL | SEQ ID NO: 111 |
| XP_011514227.1 | VVTVTGLMNQASKREKWIKADAPKGTERGIRNANQIKSIKSNQRPGTAP | SEQ ID NO: 112 |
| XP_006718027.2 | SKHDGVPVKHYDCGESGNNFRRKLCLSQLQKGDKGEKHFECNECGKAFW | SEQ ID NO: 113 |
| XP_011543232.1 | KPGPSEGDLAILGLSGGRRTLENGVNVTVSQINTVLNGRTRRYTDIQLQ | SEQ ID NO: 114 |
| XP_011535567.1 | KWLGEKTLKRPWSTSETFTAWCNSHLRKAGTQIENIEEDFRDGLKLMLL | SEQ ID NO: 115 |
| XP_011523977.1 | LGDTSWGATINYSLLRKSRSTSDLDKDDASYLRLSLGEFFAQRSEALGC | SEQ ID NO: 116 |
| XP_005258533.2 | PSPQDHDTDLTCHVDFSRKGVSAQRTVRLRVASLELQGNVIYLEVQKGQ | SEQ ID NO: 117 |
| XP_011525780.1 | QLEGGGLLRQTSRTTTAVYMLYLLSLMQPKPGAPRLQPPPNQRGLCSLA | SEQ ID NO: 118 |
| NP_001293018.1 | GSLKVHIRLHTGVRPFACPHCDKKFRTSGHRKTHIASHFKHTELRKMRH | SEQ ID NO: 119 |
| NP_659419.3 | EARALKAKEQQNISPVVEEEPEFSLPQPSLHDLTQDKLTLDTEENDVDD | SEQ ID NO: 120 |
| NP_001035095.1 | SPAPAVSTSTSSSTPSSTTSTTTTATSVAQTVSTPTTQDQTPSSAVSVA | SEQ ID NO: 121 |
| NP_059830.1 | EYQPFAGKIKDIAWTEDSKRIAVVGEGREKFGAVFLWDSGSSVGEITGH | SEQ ID NO: 122 |
| NP_001072988.1 | ALQGLENGLPTVREKTRAGSKKCRFPDDLDTNKKLKKASSKGRKSGGGP | SEQ ID NO: 123 |
| NP_001072988.1 | REAVVAKPKHKRTDYRRWKSSAEISYEEALRRARRGRRENVGLYPAPVP | SEQ ID NO: 124 |
| NP_001073996.1 | RGWILLSLCLGCFPPSERFMKYLLNFIGQGPATYGPFCAERLRRTYANG | SEQ ID NO: 125 |
| NP_001073996.1 | KATDDTTLLAFKKGDLLVLTKKQGLLASENWTLGQNDRTGKTGLVPMAC | SEQ ID NO: 126 |
| NP_060606.3 | VKKAATCLQAAYRGYKVRQLIKQQSIAALKIQSAFRGYNKRVKYQSVLQ | SEQ ID NO: 127 |
| NP_115517.1 | HEKREDQHQQKQSRDQENHYTDRDYRKERDSHRHREASHRDSHWKRHEQ | SEQ ID NO: 128 |
| NP_006653.2 | RRQPPPPPRSPFYLDSLEEKRKRQRSERLERIFQLSEAHGALAPVYGTE | SEQ ID NO: 129 |
| NP_001932.2 | PVFTEAIYNFEVLESSRPGTTVGVVCATDRDEPDTMHTRLKYSILQQTP | SEQ ID NO: 130 |
| NP_071904.4 | SREEEEISELQEDDRDQFSDQLASVGMLGRIAAEHCIPLLTSLLEERVT | SEQ ID NO: 131 |
| NP_612429.2 | RLEGDLSLADKDVTAKDSKFKMPKFKMPSYRASAPGKSIQASVDVSAPK | SEQ ID NO: 132 |
| NP_612429.2 | KDSKFKMPKFKMPSFGVSAPGKSIEASVDVSAPKVEADVSLPSMQGDLK | SEQ ID NO: 133 |
| NP_055901.2 | DGGRTKKRLDPRIYRDVLGAWGLRQGQGLLGGSPGCGAARARPEPGKGV | SEQ ID NO: 134 |
| NP_071900.2 | KELSYRSLGEDVSDSGTSKPSKPLLFSSASSQNHIPIEPDYKFSTLLMM | SEQ ID NO: 135 |
| NP_071900.2 | FENGKGPELDSVMNSENDELNGVNQVVPKKRWQRLNQRRTKPRKRMNRF | SEQ ID NO: 136 |
| NP_071900.2 | KMGKGVDGTYKKALQEAAARFEELKAQKELRQLQEDRKNDKKPPPYKHI | SEQ ID NO: 137 |
| NP_443116.2 | SCLVVFRVKICRKEARKRAAAEQDVPSTLGPISQGHQHECSAGSSQDHP | SEQ ID NO: 138 |
| NP_001128711.1 | GVHPLSCHGWVLGEHGDSSVPVWSGMNVAGVSLKTLHPDLGTDKDKEQW | SEQ ID NO: 139 |
| NP_659451.1 | ICSLQREGMGSPAYEQDLIVASRPKSFILPKLDQLSRNRGKTDRVARYF | SEQ ID NO: 140 |
| NP_663748.1 | GFERIDSNFERSSTVGKMLSNSIACYREIFHERKSQLMRKASPMSYFRK | SEQ ID NO: 141 |
| NP_002197.2 | NQWLGVSVRSQGPGGKIVTCAHRYEARQRVDQILETRDMIGRCFVLSQD | SEQ ID NO: 142 |
| NP_057735.2 | GNPLREEDRLGNHASDICGGSELDAVKTDSSLPSPSSLWSASHPSSSKK | SEQ ID NO: 143 |
| NP_001156729.1 | VGGQPCPILEPVCPEAIVCRTRPQAAPGEAAVLVVFGHAQRTLLASPFR | SEQ ID NO: 144 |
| NP_001156729.1 | GNYTSAPHCLEALQELYNHIHRYYDQIISALEEDPVGQKLQLACRLQQV | SEQ ID NO: 145 |
| NP_001161206.1 | LFRGLQNHPMVLPICSRSGDVIEYLLKNQWFVRCQEMGARAAKAVESGA | SEQ ID NO: 146 |
| NP_689711.2 | VCVTGNGMFKLLRFAEGTLKQTSFQRGEPQNYLAHTWVADDKIVVGTDT | SEQ ID NO: 147 |
| NP_733822.1 | YRFPPKFTLKAGQVVTIWAAGAGATHSPPTDLVWKAQNTWGCGNSLRTA | SEQ ID NO: 148 |
| NP_776214.1 | SGDETRLQLEAVNITDLSENRKQDKRFAFIRSDSGPTTSFESAACPGWF | SEQ ID NO: 149 |
| NP_001002912.4 | EAPVEASELSDNPGLLGEDSLKETVVPIFEATPGFEKSLENITALRKEG | SEQ ID NO: 150 |
| NP_078824.2 | GLKELVHLEWLNLAGNNLKAMEQINSCTALQHLDLSDNNISQIGDLSKL | SEQ ID NO: 151 |
| NP_004144.2 | HQVYVQILQKLTGQKATANHAAELLAKQFCTRGSPQETTVLLVDELDLL | SEQ ID NO: 152 |
| NP_001189787.1 | NIWRDPALCCYLSPGDEQVNCFNINYLRNVALVSGDTENAKGQGEQGST | SEQ ID NO: 153 |
| NP_919226.1 | RENCNEISQRVVVDSVNNQQDFKYTLIHSEENTKDATTVAEDPESANNY | SEQ ID NO: 154 |
| NP_001231681.1 | EEMITKKVAQYSQQLNDLKAENARLNSKLEKEKHNKERLEAEVESLHSN | SEQ ID NO: 155 |
| NP_001240758.1 | SHCQCKTHGVGSPGSVRQNSRTPRSPLDCGSSPKAQFLVEHETQDSKDL | SEQ ID NO: 156 |
| NP_060358.2 | ARMDKDGYFWFMGRNDDVINSSSYRIGPVEVESALAEHPAVLESAVVSS | SEQ ID NO: 157 |
| NP_001252507.1 | MACEIMPLQSAHVPQVSNVSATGELLERTIRSAVEQHLFDVNNSGGQSS | SEQ ID NO: 158 |
| NP_054733.2 | AQLTDKTLNLCKMIDKRMWQSMCPLRQFRKLPEEVVKKIEKKNFPFERL | SEQ ID NO: 159 |
| NP_001258901.1 | YAWESSAGGSFTVRADHGEPIGRGTKVILHLKEDQTEYLEERRVKEVVK | SEQ ID NO: 160 |
| NP_000884.1 | CVHPISTQSPDLEPILRHGIQYFNNNTQHSSLEMLNEVKRAQRQVVAGL | SEQ ID NO: 161 |
| NP_003451.1 | SRGAMDTKGHKTAGDVGSKAVAAVAAFAGVVATLGFIYYLYEKRTVSNH | SEQ ID NO: 162 |
| NP_005558.1 | PYQSFQTPQHPSFLFQDKRVSWSLVYLPTIQSCWNYGFSCSSDELPVLG | SEQ ID NO: 163 |
| XP_005271379.1 | TEEERKRRIEQDMLEKRKIQRELAKRAEQIEDINNTGTESASEEGDDSL | SEQ ID NO: 164 |
| XP_005264182.1 | SAASFSGHATPSQQPEPVVHSLKVVWRKLGDAAGSCPGIRQHLSGNQYK | SEQ ID NO: 165 |
| XP_005264966.1 | FETKPLDAFRDKVQVGHLQRVDPQDGEGHLSSDSSSALPFSQSAPQRDE | SEQ ID NO: 166 |
| XP_005264966.1 | KISVTVSSSARPSGSGQEVGGQTAVKNQAKVECHTEAQSQVKIRNHTEA | SEQ ID NO: 167 |
| XP_005264966.1 | VQPEPAPPSASPLPRGWQKSVLELQTGPGSSQHYGAMRTVTEQYEEVDQ | SEQ ID NO: 168 |
| XP_005247674.1 | TLSKKVKIVATNLNAAEEALEAYQREKQQRLNELLVVIPLKLHQIEYVV | SEQ ID NO: 169 |
| XP_005247674.1 | KQLIREKREMTKTIHKMEETVRQLMISKFGRVVNLEALQTLSVNTTLEE | SEQ ID NO: 170 |
| XP_005250082.1 | YISVTPKIGMGKPAITKRKFSPGRPRSKQGAWSTHNTVSPPSWSPDISE | SEQ ID NO: 171 |
| XP_005250088.1 | SPPIAFPPAFEAAQVEAKPDELKVTVKLKPRLRAVHGGFEDCRPLNKKW | SEQ ID NO: 172 |
| XP_005270318.1 | MPFIDVPTPISSASSEAASAVVSPSTDSGLEFSSQTTSKEDLTDLEQPG | SEQ ID NO: 173 |
| XP_005274302.1 | DLKGPEADIKGPKVDINTPDVDVHGPDWHLKMPKVKMPKFSMPGFKGEG | SEQ ID NO: 174 |
| XP_005271661.1 | RHHPWRHGLFQMRKLRCKAVKQLVQDLDQVLNHPTPHATVPNPCDSSFP | SEQ ID NO: 175 |
| XP_005266358.1 | REREKERDRERDRDRDHDRERERERERDREKEREREREERERERERERE | SEQ ID NO: 176 |
| XP_005266358.1 | RAQDIIGHHQSEDRETSDRAHDENKKKAKIQKKPIKKKKEDDVGIERGN | SEQ ID NO: 177 |
| XP_005266358.1 | RGPRTPPITTKEELVEMCNGKNGILEDSQKKEDTAFSDWSDEDVPDRTE | SEQ ID NO: 178 |
| XP_005259118.1 | MAQLWAAEAALLPQNDTRLDPEAYGSPCARGSQPWQVSLFNGLSFHCAG | SEQ ID NO: 179 |
| NP_612114.2 | DFQESGVSEEVSESEDEQRPRTRSAKKAELEENQRSYKQKKKRRRIKVQ | SEQ ID NO: 180 |
| NP_612114.2 | KKKRDTPMLPKDTILAELLQIHKEHIVGYHEHDSLLDHKEEEELTEEER | SEQ ID NO: 181 |
| NP_775871.2 | GTTGPSAGVTETTGSSAGVAGTTRLSAGVTGITGLSAGVTGTTGLSTEV | SEQ ID NO: 182 |
| NP_775871.2 | GLSAGVTGTTGLSPGVTGTSGLSAEVTGTTGPSAEATGLPGVSAGVTGT | SEQ ID NO: 183 |
| NP_775871.2 | VLKSQCDCYINDEVMQPGKLIHIDDNKCVCRDGILLCQIPIDLTLQNCS | SEQ ID NO: 184 |
| NP_005103.2 | GDPKGNNFLYTNGKCVILRNIDDHSRFVNCVRFSPDGNRFATASADGQI | SEQ ID NO: 185 |
| NP_001269388.1 | MGPLMVLFCLLFLYPGLADSAPSCPQNVNISGGTFTLSHGWAPGSLLTY | SEQ ID NO: 186 |
| NP_006120.1 | AGCDHKVTSTSGTITSPNWPDKYPSKKECTWAISSTPGHRVKLTFMEMD | SEQ ID NO: 187 |
| NP_066933.1 | VVYYPTRGGKPDEVVVDGTVSSTVLKNLMSLTEYQIAVFAIYAHTASEG | SEQ ID NO: 188 |
| NP_004548.3 | GTCQCAPGFLGETCQFPDPCQNAQLCQNGGSCQALLPAPLGLPSSPSPL | SEQ ID NO: 189 |
| NP_004548.3 | ECLDQPCHPTGTAACHSLANAFYCQCLPGHTGQWCEVEIDPCHSQPCFH | SEQ ID NO: 190 |
| NP_003557.2 | LQKVKMEKEALMTELSTVKDKLSKVSDSLKNSKSEFEKENQKGKAAILD | SEQ ID NO: 191 |
| NP_003557.2 | QEQLIQAQNTLKQNEKEEQQLQGNINELKQSSEQKKKQIEALQGELKIA | SEQ ID NO: 192 |
| XP_006710413.1 | DNPGLLGEDSLKETVVPIFEATPGFEKSLENITALRKEGGGERLSEARD | SEQ ID NO: 193 |
| XP_006712126.1 | PSEKIPHLGTLAMLDWQNGSMGRSGEALLLDTAHVLFTLSSQIKEPTLE | SEQ ID NO: 194 |
| XP_006712126.1 | LPPAAEFVKTVEFTRLRIQEERTLACLRLLAFLEGKEGEDTLVLSVWDS | SEQ ID NO: 195 |
| XP_006712127.1 | CHLQRMVSEQCHLLSQFFRELPPAAEFVKTVEFTRLRIQEERTLACLRL | SEQ ID NO: 196 |
| XP_006713759.1 | AGFQAAIGENNKFANFLMKVLKKKIKRVKKKEVEGDADEDEESEESSEE | SEQ ID NO: 197 |
| XP_006713806.1 | QVNSWVGINSNDDQLFAVKNNFPASVHTTRYSRNDLHLEDIQTDEDKLN | SEQ ID NO: 198 |
| XP_006713806.1 | VQQLEDADERTNFDTETRDSKLHIACFPVQLDTLSDGASVDESHGISPP | SEQ ID NO: 199 |
| XP_006713806.1 | SQGKGRAYRPGQHIQLVQYLATVCPLTSTLGLQTAEDAKLEKILSKQRF | SEQ ID NO: 200 |
| XP_006714871.1 | KVKVEDAGEYVCEAENILGKDTVRGRLYVNSVSTTLSSWSGHARKCNET | SEQ ID NO: 201 |
| XP_006717798.1 | QALEQLKPLLSLLKVAGREGNLIAIFHEEHSRVLQQLQVFTFFPESLPA | SEQ ID NO: 202 |
| XP_006718114.1 | QTVIQDFEKPESPRAASSSSPGSPVASSPSRLSPTPDGPPPLAQPPLIL | SEQ ID NO: 203 |
| XP_006718732.1 | LTSALSIQNYHLECTETQAWMREKTKVIESTQGLGNDLAGVLALQRKLA | SEQ ID NO: 204 |
| XP_006718732.1 | RLEALGTGWEELGRMWESRQGRLAQAHGFQGFLRDARQAEGVLSSQEYV | SEQ ID NO: 205 |
| XP_006718732.1 | LQKAYAGDKAEEIGRHMQAVAEAWAQLQGSSAARRQLLLDTTDKFRFFK | SEQ ID NO: 206 |
| XP_006723899.1 | SKDNFETFLFATVSNREQEDLCRGIVQLCFNEQSQQLLAEVQPSDSFLM | SEQ ID NO: 207 |
| XP_005262214.2 | NTALNWMNEFEKWQEGLKDDEKLEVSELATVKRPQERSYMLQRWQEDGG | SEQ ID NO: 208 |
| XP_005262214.2 | EYEAEKKGLTMRFNIPTGTNLPPVSFNSQTPYIPFNLGALSAMSNQQLE | SEQ ID NO: 209 |
| NP_008877.1 | REEEERRKQPPAPEPTASVPPGDLVGGQTASDTTWDGTQPRPPPSTQAP | SEQ ID NO: 210 |
| NP_009187.1 | MGPRIGPAGEVPQVPDKETKATMGTENTPGGKASPDPQDVRPSVFHNIK | SEQ ID NO: 211 |
| NP_683515.4 | KRIEKLEEYTKTCMDCLKEKKQQEETLVDEIEKTKSRMSEVNEELNLIR | SEQ ID NO: 212 |
| NP_001611.1 | PDVDVHGPDWHLKMPKVKMPKFSMPGFKGEGPEVDVKLPKADVDVSGPK | SEQ ID NO: 213 |
| NP_001611.1 | ECPDAKLKGPKFKMPEMHFKTPKISMPDVNLNLKGPKVKGDMDVSVPKV | SEQ ID NO: 214 |
| NP_001013653.1 | RQRLSGNTSRRGKSGFNSKSGKRGSSKSGKLKGDDLQAIKQELTQIKQK | SEQ ID NO: 215 |
| NP_001017995.1 | SEISLSVGQVVDIIEKNESGWWFVSTAEEQGWVPATCLEGQDGVQDEFS | SEQ ID NO: 216 |
| NP_001289481.1 | VRHVINFDLPSDIEEYVHRIGRTGRVGNLGLATSFFNEKNMNITKDLLD | SEQ ID NO: 217 |
| NP_001028725.1 | EKASESDAEIKDMKETIFELEDQVEQHRAVKLHNNQLISELESSVIKLE | SEQ ID NO: 218 |
| XP_011508532.1 | GSKLHFLENGNNKAASVQEEFMDAVCDGVGLGMKILLDSGLEKAKELQH | SEQ ID NO: 219 |
| XP_011508535.1 | HIVHQELESLAKSLLFCFESEESPDLLKPWETYNQNTKEEHQQSKSSGI | SEQ ID NO: 220 |
| XP_011508536.1 | SPAPSILKNRMSNLQVKQRPKSSFLANKQERSAENTILPEEETVVQNTS | SEQ ID NO: 221 |
| XP_011508536.1 | SRRRSRSLMKNRRISGCLHDIQVHPIKNLHSSHSSGLMDKSSTIYSNSA | SEQ ID NO: 222 |
| XP_011531004.1 | LLQSKLNQIFEITIRPPPSPSGTITAAYGQPQNHSIPVYEMKFPDLCVY | SEQ ID NO: 223 |
| XP_011509143.1 | SATNVASTPDNRGRSRAKVVSQSQRSRSANPAGAGSRSSSPGKLLGSGY | SEQ ID NO: 224 |
| XP_011509520.1 | FEVITLSGPRLPKTQLILAVNWKGLCFLDQQEKMLLELSFPEVMGLATN | SEQ ID NO: 225 |
| XP_011509520.1 | NREAQGGQRLLLSTMHEEYEFVSPSSVAIAELVALFLEGLKERSIFAMA | SEQ ID NO: 226 |
| XP_011509520.1 | ALQHPALQDEVYCQILKQLTHNSNRHSEERGWQLLWLCTGLFPPSKGLL | SEQ ID NO: 227 |
| XP_011510516.1 | HNPACESVIDSSAFGEGKAPGPPFPQTLGIANVATRLSSIQLGQSEKER | SEQ ID NO: 228 |
| XP_011510516.1 | SGKSLASAISGKIPSPLSSSYKPMLNNNSFMWPNSTPVPLLQTTQGLKP | SEQ ID NO: 229 |
| XP_011511210.1 | AYDYRGRTVVFAGTRSGRIRKILVDLSNPGGRPALAYESVVAQEGSPIL | SEQ ID NO: 230 |
| XP_011511210.1 | GQGDQRVVKLYLKSKETGKKFASVDFVFYNCSVHQSCLSCVNGSFPCHW | SEQ ID NO: 231 |
| XP_011511210.1 | SPARVTALRFNSSSLQCQNSSYSYEGNDVSDLPVNLSVVWNGNFVIDNP | SEQ ID NO: 232 |
| XP_011536015.1 | SWSGHARKCNETAKSYCVNGGVCYYIEGINQLSCKAPGLHCLELGTQSH | SEQ ID NO: 233 |
| XP_011532914.1 | RRGKIPENLGLNFISGDISDTQASNELSRIANSLTGSNTAPGSFLFSSC | SEQ ID NO: 234 |
| XP_011514753.1 | VHCLPQLPEKASPPASPPIAFPPAFEAAQVEAKPDELKVTVKLKPRLRA | SEQ ID NO: 235 |
| XP_011514753.1 | LDPLLSSSSAPTKSGTHGPADDPLADISEVLNTDDDILGIISDDLAKSV | SEQ ID NO: 236 |
| XP_011514753.1 | VSQLPGPVPTSGVTDTQNTVNMAQADTEKLRQRQKLREIILQQQQQKKI | SEQ ID NO: 237 |
| XP_011514754.1 | TSTSKSFQSTVTGELNAPYSKQFVHSKSSQYRKMKTEWKSNVYLARSRI | SEQ ID NO: 238 |
| XP_011514754.1 | PALTPRPIDNFSHNQSNDPYSQPPLTPHPAVNESFAHPSRAFSQPGTIS | SEQ ID NO: 239 |
| XP_011514754.1 | TNQRHSDPYAHPPGTPRPGISVPYSQPPATPRPRISEGFTRSSMTRPVL | SEQ ID NO: 240 |
| XP_011542890.1 | NDFTNETKISPIDNYIVLHQEYKESESHNSFGKSCDKILITQELEITKS | SEQ ID NO: 241 |
| XP_011542890.1 | EPVSLENIQRDYKETAYVEDRGQDHNLFCNSQLSNDIWLNVNFKKQTDR | SEQ ID NO: 242 |
| XP_011542890.1 | SSTGNDCDATCIGHTKAKTDVLISVLDSNVKHFLNDLYQQGNLILSDCK | SEQ ID NO: 243 |
| XP_011515869.1 | CCRAIVKVFLALAAKGPVLRNCISTVVHQGLIRICSKPVVLPKGPESES | SEQ ID NO: 244 |
| XP_011516544.1 | NMKVYFYNFGWKDYGVASLTTILDMVKVMTFALQEGKVAIHCHAGLGRT | SEQ ID NO: 245 |
| XP_011537862.1 | VYTFKARNPNELSVSANQKLKILEFKDVTGNTEWWLAEVNGKKGYVPSN | SEQ ID NO: 246 |
| XP_011538616.1 | VLENRDRVGCVWQTVRDHLYHLCVQAQDFCFLVERAVVGLLRLAIRLLR | SEQ ID NO: 247 |
| XP_011538616.1 | GDDWATLFTLLECIGSGVKPPAALQATARADAPDAGAQSDSELPSYHQN | SEQ ID NO: 248 |
| XP_011518459.1 | SISKCMEMKNTILARQKEMHSSLEKVKQLIRLIHGIDLSKPVDSEATVG | SEQ ID NO: 249 |
| XP_011543231.1 | VAGAPSGCDCKNDANCDCFSGDDGYAKDAKLNTPSSLAVCADGELYVAD | SEQ ID NO: 250 |
| XP_011543232.1 | KSNENGWTTFYEYDSFGRLTNVTFPTGQVSSFRSDTDSSVHVQVETSSK | SEQ ID NO: 251 |
| XP_011543232.1 | TLQDNLIEMDILGASRHDGAYSDGHFLFKPGGTSPLFCTTSPGYPLTSS | SEQ ID NO: 252 |
| XP_011537116.1 | VAVLKKELVQVQTLMDNMTLERERESEKLKDECKKLQSQYASSEATISQ | SEQ ID NO: 253 |
| XP_011544284.1 | QQPKSLKVRAGQRPPGLPLPIPGALGPPNTGTPIEQQPCSPGQEAVLDQ | SEQ ID NO: 254 |
| XP_011523431.1 | HLDLLLVVGMGGVAALFGMPWLSATTVRSVTHANALTVMGKASTPGAAA | SEQ ID NO: 255 |
| XP_011523974.1 | SPLEQAQDSPIDFHLQSWMNNKEPKIVVLDAGKHFEDKTLKSDLSHTSL | SEQ ID NO: 256 |
| XP_011523977.1 | VISSGSKPLSPGPCLDIPSILSNKQFLAWGGVPLGRTQLQKLALRNNSA | SEQ ID NO: 257 |
| XP_011525583.1 | CGQVLNPKISHNEQQRIPFEESQYKCSETSHSSSLTQNMRNNSEEKPFE | SEQ ID NO: 258 |
| XP_011525624.1 | ELEEEQSNSELLNDRYRKLLLQVESLTTELSAERSFSAKAESGRQQLER | SEQ ID NO: 259 |
| XP_011525782.1 | CQEMASVLGTNPHLVELDLTGNALEDLGLRLLCQGLRHPVCRLRTLWLK | SEQ ID NO: 260 |
| XP_011525784.1 | EDLGLRLLCQGLRHPVCRLRTLWLDSCGLTAKACENLYFTLGINQTLTD | SEQ ID NO: 261 |
| XP_011527330.1 | PQEKSEGTYCCGPVPVRAIKEGDLSTKYDAPFVFAEVNADVVDWIQQDD | SEQ ID NO: 262 |
| XP_011528036.1 | NKAGLELWGSRTSSVCKSRQFLHLTVVESRGSEKAPVTTLACLQGCLGK | SEQ ID NO: 263 |
| XP_011528036.1 | YPNCPVTRFPVPNEKVPWETEFLIYDPPFYTAERKDAAAMDPMGDTLEP | SEQ ID NO: 264 |
| NP_001297064.1 | GNSYMEYGHDEATKQAQKDFLKALHINPAYIKARISFGYNLQAQGKFQK | SEQ ID NO: 265 |
| NP_659419.3 | NQINVPALDTVVTPDDVRYFTNETDDIANLEASVLENPSHVQLWLKLAY | SEQ ID NO: 266 |
| NP_659419.3 | LWKDQLLFEASEGGKTDNLRKLVSKCQEIGVSLNELLNLNSNKTESKNH | SEQ ID NO: 267 |
| NP_059830.1 | YGHHAKIVCLAWSPDNEHFASGGMDMMVYVWTLSDPETRVKIQDAHRLH | SEQ ID NO: 268 |
| NP_061833.1 | MVDSVYRTRSLGVAAEGLPDQYADGEAARVWQLYIGDTRSRTAEYKAWL | SEQ ID NO: 269 |
| NP_002461.2 | GLDETIAKLTREKKALQEAHQQALDDLQAEEDKVNSLNKTKSKLEQQVE | SEQ ID NO: 270 |
| NP_003793.2 | QTLDDLQVEEDKVNGLIKINAKLEQQTDDLEGSLEQEKKLRADLERAKR | SEQ ID NO: 271 |
| NP_003793.2 | RKLEGDLKMSQESIMDLENDKQQIEEKLKKKEFELSQLQAKIDDEQVHS | SEQ ID NO: 272 |
| NP_000055.2 | RMNKTVAVRTLDPERLGREGVQKEDIPPADLSDQVPDTESETRILLQGT | SEQ ID NO: 273 |
| NP_000055.2 | QYFNVELIQPGAVKVYAYYNLEESCTRFYHPEKEDGKLNKLCRDELCRC | SEQ ID NO: 274 |
| NP_000055.2 | CTRFYHPEKEDGKLNKLCRDELCRCAEENCFIQKSDDKVTLEERLDKAC | SEQ ID NO: 275 |
| NP_057613.4 | LLGATAIEDRLQAGVPETIATLLKAEIKIWVLTGDKQETAINIGYSCRL | SEQ ID NO: 276 |
| NP_001072988.1 | YQCDLVSKNGNDVYRYPSPLHAVAVQSPMFLLCLTGNPLREEDRLGNHA | SEQ ID NO: 277 |
| NP_001073996.1 | AELVALFLEGLKERSIFAMALQDRKATDDTTLLAFKKGDLLVLTKKQGL | SEQ ID NO: 278 |
| NP_001073996.1 | LLASENWTLGQNDRTGKTGLVPMACLYTIPTVTKPSAQLLSLLAMSPEK | SEQ ID NO: 279 |
| NP_001073996.1 | PLARARGHLWAYSCEPLRQPLLKRVHANVDLWDIACQIFVAILRYMGDY | SEQ ID NO: 280 |
| NP_001074248.1 | LGLLENLRVRRAGFAYRRKYEAFLQRYKSLCPETWPTWAGRPQDGVAVL | SEQ ID NO: 281 |
| NP_060606.3 | QYCFYQKLQWATKIIQEKYRANKKKQKVFQHNELKKETCVQAGFQDMNI | SEQ ID NO: 282 |
| NP_060606.3 | YLKTKAAVVTLQSAYRGMKVRKRIKDCNKAAVTIQSKYRAYKTKKKYAT | SEQ ID NO: 283 |
| NP_115517.1 | QEDKPRARDQRERSDRVWKREKDREKYSQREQERDRQQNDQNRPSEKGE | SEQ ID NO: 284 |
| NP_115517.1 | KDRKPKYIHNLLKAVEIRKKEQEKRMEKKIQREREMEKGEFDDKEAFVT | SEQ ID NO: 285 |
| NP_006653.2 | GARAETQGANHTPVISAHQTRSTTTPPRCSPARERVPRPAPRPRPTPAS | SEQ ID NO: 286 |
| NP_006653.2 | PITILPVHILPSPPPPSQIPPCSSPACTPPPACTPPPAHTPPPAQTCLV | SEQ ID NO: 287 |
| NP_006653.2 | CLGSSSAASSPPPPASRLDDEDGDFQPQEDEEEDDEETIEVEEQQEGND | SEQ ID NO: 288 |
| NP_006653.2 | KTIQTISLLAHLACEKGNWGPHLIIVPTSVMLNWEMELKRWCPSFKILT | SEQ ID NO: 289 |
| NP_001932.2 | TSKILDREVETPKNELYNITVLAIDKDDRSCTGTLAVNIEDVNDNPPEI | SEQ ID NO: 290 |
| NP_071904.4 | EDSEAVGISSIISNLITVFPRNVLTAIPSELFSSFVNCLTHLTCSFGRS | SEQ ID NO: 291 |
| NP_071904.4 | DINTTLQILGSPGEKASSIPGYNRTDSVIRLLSAILRVSEVESRAIRAD | SEQ ID NO: 292 |
| NP_149015.2 | PVDLTAGRRAVCCDVVYKLPFGRSCTAQQPATTLPEDRFGYRDDHYQYD | SEQ ID NO: 293 |
| NP_149015.2 | IPLTSKTYEEVQSIISQQSGEAEICVRLDLNMLSDSENSQHLELHEPPK | SEQ ID NO: 294 |
| NP_149015.2 | DFLGEVLIDLSSTSHLDNTPRWYPLKEQTESIDHGKSHSSQSSQQSPKP | SEQ ID NO: 295 |
| NP_149015.2 | GISSSLGEIPSLIPTDEKDILKGLKKDSFSQESSPSSPSDLAKLESTVL | SEQ ID NO: 296 |
| NP_149015.2 | SLILTYSEPSESATSVPPSDTPSLTSSVSSVCTTDSSSPITTLDSITTV | SEQ ID NO: 297 |
| NP_149015.2 | PGSIIDYPEEISVSLDRTAPPDGRASADHIVISLSDMASSIIESVVPKP | SEQ ID NO: 298 |
| NP_036336.2 | MNTADQARVGPADDGPAPSGEEEGEGGGEAGGKEPAADAAPGPSAAFRL | SEQ ID NO: 299 |
| NP_001095886.1 | EKKIYPTVNCQPLGMISLMKRPPGFSPFRSSRIGEIKEETTVSPPHTSM | SEQ ID NO: 300 |
| NP_612429.2 | EASVDVSELKVEADGSFPSMQGDLKTTDIRIQPPSAQLEVQAGQVDVKL | SEQ ID NO: 301 |
| NP_612429.2 | KVDRKGPQIDVKGPKLDLKGPKTDVTAPDVEVSQPGMEVDVEAPGAKLD | SEQ ID NO: 302 |
| NP_612429.2 | MPKFKMPSFGMLSPGKSIEVSVDVSAPKMEADMSIPSMQGDLKTTDLRI | SEQ ID NO: 303 |
| NP_612429.2 | CKESPGLWGASILKAGAGVPGEQPVDLNLPLEAPPISKVRVHIQGAQVE | SEQ ID NO: 304 |
| NP_612429.2 | VDLNLPLEAPPISKVRVHIQGAQVESQEVTIHSIVTPEFVDLSVPRTFS | SEQ ID NO: 305 |
| NP_612429.2 | KKQEKAGWFRFPKLGFSSSPTKKSKSTEDGAELEEQKLQEETITFFDAR | SEQ ID NO: 306 |
| NP_612429.2 | TFFRMSRPEAVQEATEVTLKTEVEAGASGYSVTGGGDQGIFVKQVLKDS | SEQ ID NO: 307 |
| NP_612429.2 | DKDVTAKDSRFKMPKFKMPSFGASAPGKSIEASVDVSAPKVEADVSLPS | SEQ ID NO: 308 |
| NP_612429.2 | PSFKVPKVDLKGPEIDIKGPKLDLKDPKVEVTAPDVEVSLPSVEVDVEA | SEQ ID NO: 309 |
| NP_612429.2 | SLADKDVTAKDSKFKMPKFKMLSFGVSALGKSIEASADVSALKVEADVS | SEQ ID NO: 310 |
| NP_219499.1 | GWTAAHIAASKGFKNCLEILCRHGGLEPERRDKCNRTVHDVATDDCKHL | SEQ ID NO: 311 |
| NP_065800.1 | PRSLCIQPQTAPDALPPEKTLELTQYKTKCENQSGFILQLKQLLACGNT | SEQ ID NO: 312 |
| NP_001119522.1 | LLSKGGRRAQIPDVSRATPHTIDEMPEAAVKSTANKYQVFFFGTHETAF | SEQ ID NO: 313 |
| NP_060547.2 | KGVHGNMKGQSLGYAFAEFQEHEHALKALRLINNNPEIFGPLKRPIVEF | SEQ ID NO: 314 |
| NP_006288.2 | SPKGKRKLDLNQEEKKTPSKPPAQLSPSVPKRPKLPAPTRTPATAPVPA | SEQ ID NO: 315 |
| NP_065708.2 | YDGQMLFSCGDEGKAFLDTFTLLDSQMTHAEVRPFRCLPCGNVFKEKSA | SEQ ID NO: 316 |
| NP_055901.2 | PRRRWDRPPPYVAPPSYEGPHRTLGTKRGPGNSQVPTSSAPAATPARTD | SEQ ID NO: 317 |
| NP_001123633.1 | KPFQKPRSLDSSRIFDGKAKEPYAEQPNDKMEAESIYINDPTVKPINDD | SEQ ID NO: 318 |
| NP_001123633.1 | HSHFDKLGETKQTPHSLPSRGASFSDRTPSACRLVDNTIHQFQNLGLLD | SEQ ID NO: 319 |
| NP_001123633.1 | RTQSLGSNNSVILDGLKRRQNFLQNVEGTKSSQPLTSNSLLPLTPVINV | SEQ ID NO: 320 |
| NP_071900.2 | GGAALKENVCQNCEKLGELLLCEAQCCGAFHLECLGLTEMPRGKFICNE | SEQ ID NO: 321 |
| NP_071900.2 | DTNQMLSLSKKALAGTCQRPLLPERPLERTDSRPQPLDKVRDLAGSGTK | SEQ ID NO: 322 |
| NP_071900.2 | RPLERTDSRPQPLDKVRDLAGSGTKSQSLVSSQRPLDRPPAVAGPRPQL | SEQ ID NO: 323 |
| NP_443116.2 | LEHGGGLGLGAALGAGVAALLAFCSCLVVFRVKICRKEARKRAAAEQDV | SEQ ID NO: 324 |
| NP_001128711.1 | KNRVIGSGCNLDSARFRYLMGERLGVHPLSCHGWVLGEHGDSSVPVWSG | SEQ ID NO: 325 |
| NP_071896.8 | PEKELIHLLPIKDKSGIIPQTREKPVTDSNKDEEDQEEERELEEEIIED | SEQ ID NO: 326 |
| NP_004416.2 | QHPNEQKEGTPAPFGDQSHPEPESWNAAQHCQQDRSQGGWGHRLDGFPP | SEQ ID NO: 327 |
| NP_002197.2 | DADTDRRLRGQVPRVTFLSRNLEEPKHQASGTVWLKHQHDRVCGDAMFQ | SEQ ID NO: 328 |
| NP_002197.2 | LVRARFCTRVSDTEFQPLPMDVDGTTALFALSGQPVIGLELMVTNLPSD | SEQ ID NO: 329 |
| NP_002197.2 | HVECELGNPMKRGAQVTFYLILSTSGISIETTELEVELLLATISEQELH | SEQ ID NO: 330 |
| NP_001138468.1 | GGQGPGQKGLCSPRPNILHLDVDSRDRRRRELEPPEQQEPGERQEPSMS | SEQ ID NO: 331 |
| NP_001138994.1 | EEKAAMTDAMVPRSSYEKLQSSLESEVSVLASKLKESVKEKEKVHSEVV | SEQ ID NO: 332 |
| NP_001138994.1 | SEVSVLASKLKESVKEKEKVHSEVVQIRSEVSQVKREKENIQTLLKSKE | SEQ ID NO: 333 |
| NP_001138994.1 | LKEALNSLSQLSYSTSSSKRQSQQLEALQQQVKQLQNQLAECKKQHQEV | SEQ ID NO: 334 |
| NP_001139281.1 | ELERQLLQANPILEAFGNAKTVKNDNSSRFGKFIRINFDVAGYIVGANI | SEQ ID NO: 335 |
| NP_001139281.1 | CRLLGLGVTDFSRALLTPRIKVGRDYVQKAQTKEQADFALEALAKATYE | SEQ ID NO: 336 |
| NP_004245.2 | TASDLFRIPMLRRMTFCLSLAWFATGFAYYSLAMGVEEFGVNLYILQII | SEQ ID NO: 337 |
| NP_004245.2 | TQAAALLLAGGAILALTFVPLDLQTVRTVLAVFGKGCLSSSFSCLFLYT | SEQ ID NO: 338 |
| NP_001156729.1 | MLRRTETMVEKLLTNWLSICLYAFLREVAGEPLYMLFRAIQYQVDKGPV | SEQ ID NO: 339 |
| NP_000681.2 | NYLSQALQAGTVWVNCYDVFGAQSPFGGYKMSGSGRELGEYGLQAYTEV | SEQ ID NO: 340 |
| NP_001161206.1 | EMGARHGLSPLNVIAEDGTMTSLCGDWLQGLHRFVAREKIMSVLSEWGL | SEQ ID NO: 341 |
| NP_689711.2 | LPSYEQMVAASSHSQMSMPQVFAIAAYSKGFACSAGPGRVLLFEKMEEK | SEQ ID NO: 342 |
| NP_689711.2 | AAYSKGFACSAGPGRVLLFEKMEEKDFYRESREIRIPVDPQSNDPSQSD | SEQ ID NO: 343 |
| NP_689711.2 | KSCSYNCVTVSPDAKIIFAVGSDHTLKEIADSLILREISAFDVTYTAIV | SEQ ID NO: 344 |
| NP_689711.2 | KSCSYNCVTVSPDAKIIFAVGSDHTLKEIADSLILREISAFDVTYTAIV | SEQ ID NO: 345 |
| NP_776214.1 | YLRNNQLVAGYLQGPNVNLEEKIDVVPIEPHALFLGIHGGKMCLSCVKS | SEQ ID NO: 346 |
| NP_776214.1 | KRFAFIRSDSGPTTSFESAACPGWFLCTAMEADQPVSLTNMPDEGVMVT | SEQ ID NO: 347 |
| NP_001002912.4 | FVCVERSSPCYKCIIAMGLDKKPSLPKSRKEKSTEKGEELKKAEGKVRK | SEQ ID NO: 348 |
| NP_001002912.4 | KEREYVIPKRNEIKENKTSVSAKFSAQEIKTGLKEVVTAVEEMTSKGKP | SEQ ID NO: 349 |
| NP_001002912.4 | PGQEVLEDDQENTLKYEYEEDFEVDEEKQGEKSNEEGQADVQMNGIPQS | SEQ ID NO: 350 |
| NP_001002912.4 | KEVMRTETRLSPFTGEAEASRMQVSEGSPEEGSLAKEAFLCKEDVEGEE | SEQ ID NO: 351 |
| NP_001002912.4 | NRALKEGHRQDGEGALAAPEAEPAGKVQAPEGLIPATGQAEELAAKDHD | SEQ ID NO: 352 |
| NP_001167594.1 | TYPLSCIRSQPMQFFPRVDSEVVLKSFLSDLKSKLPHICGFPIKMTSKP | SEQ ID NO: 353 |
| NP_001167594.1 | KRKLCPESSKTSKKHHSDTVHYGQSSSSKKQILDSDKSKLKKSLIIHNA | SEQ ID NO: 354 |
| NP_005886.2 | LSPDTDDRLENTSLAGDSVSEVDGNDSDSSSYSSASTRGTYGILSKTVG | SEQ ID NO: 355 |
| NP_005886.2 | NLQVQAVLQRKEEEDRQMKHLVQALQASLEKEKEKVNSLKEQVAAAKVE | SEQ ID NO: 356 |
| NP_705616.2 | ARESDGWYVLEKVDLHPQLCFKFSFGNSSHVECPHQTGSLTSWNVSMDT | SEQ ID NO: 357 |
| NP_705616.2 | PGLGQDTLVPPVYTVSQARGSSPVSLDLIIPFLRPGCCVLVWRSDVQFA | SEQ ID NO: 358 |
| NP_705616.2 | RAALGGGRDVIVDLWEGRHVARVGPLPWLWAARTRVAREQGTVLLLWSG | SEQ ID NO: 359 |
| NP_001180432.1 | NPDLILPVPAFNVINGGSHAGNKLAMQEFMILPVGASSFKEAMRIGAEV | SEQ ID NO: 360 |
| NP_078824.2 | NSLLEQYLTSVQQLEDADERTNFDTETRDSKLHIACFPVQLDTLSDGAS | SEQ ID NO: 361 |
| NP_078824.2 | FDYRPYIVSWCLNLRVLDGYVISQKESLKAEWLYSQGKGRAYRPGQHIQ | SEQ ID NO: 362 |
| NP_056097.1 | GFPPLDRFGLGQPGRIPGSVNAMRVLSTSTDLEAAVADALKKPVRRRYE | SEQ ID NO: 363 |
| NP_056097.1 | VLSTSTDLEAAVADALKKPVRRRYEPYGMYSDDDANSDASSVCSERSYG | SEQ ID NO: 364 |
| NP_004144.2 | EHRIILRTRIAASKTIDIREERTLTPISGGQRSSVVPSVILKPENIKKR | SEQ ID NO: 365 |
| NP_004144.2 | YPTMSETMAVCSHLGSCRLLLVEPSRNDLLLRVRLNVSQDDVLYALKDE | SEQ ID NO: 366 |
| NP_001193775.1 | EIREENKLYKRTALALHYLLTYKHLSAILEALKHLEVVTRLSPLCCENM | SEQ ID NO: 367 |
| NP_976054.2 | MTRTQKGKTFLPHCFYQSLPPRLGWGMILNYSKLKGKEECASVSSVPMV | SEQ ID NO: 368 |
| NP_976054.2 | GELKLMTAKLLPSFLHCFSDDFTAVRRAACLAAGALQIRDKMVLECLLN | SEQ ID NO: 369 |
| NP_976054.2 | ACRSILALKLQGDRVRDTFLDVLLLENHDAVLKEMYQTMKILNLGNEGN | SEQ ID NO: 370 |
| NP_976054.2 | KDLRTSPEKRIAVGPFRSDYPALYLGKFSERTFFSPIMSSPSGKKGAHL | SEQ ID NO: 371 |
| NP_001182760.2 | TLPQMEAELENFHKQNTQLELNITELWQKLRATDQEMRRERQKERDLEA | SEQ ID NO: 372 |
| NP_006477.2 | FQCSCPSSGYRLAPNGRNCQDIDECVTGIHNCSINETCFNIQGGFRCLA | SEQ ID NO: 373 |
| NP_919226.1 | SVGKGFSRKSRFVPSACHLQQSSPTDVLLSSEEKTNSFHPPEAMCRDKE | SEQ ID NO: 374 |
| NP_919226.1 | MCKTWNTEYNTYDTISSKNHCKKNTILLNGQSNATMIHSGKHNLTYSRT | SEQ ID NO: 375 |
| NP_919226.1 | QPNMLANKVKFTFPPAALPPPSTPLQPLPLQQSLCSTSVTTIHHTVLQQ | SEQ ID NO: 376 |
| NP_919226.1 | PRLCPGNQPTFVAPPQMPIIPASVLHPSHLAFPSLPHALFPSLLSPHPT | SEQ ID NO: 377 |
| NP_001231681.1 | LKRGANPNIKDIYGNTALHYAVYNEGTSLAERLLSHHANIEALNKEGNT | SEQ ID NO: 378 |
| NP_001238972.1 | PPPTHLRPCPASQLPGLLSHGLLAGLSFAVGSSSGLLPLLLLLLLPLLA | SEQ ID NO: 379 |
| NP_001240758.1 | LCVREFTQFLTPLRNIFSCCDPKAHAVTLPQYLIRQRHLLHGYEARLLK | SEQ ID NO: 380 |
| NP_001240758.1 | NLLEQGETPQTVPAQILVGHKPRQQKLISHCYIPQSPEPDLHKEALVRS | SEQ ID NO: 381 |
| NP_001244303.1 | RERDTSRRLLAEKEREMAEMRARMQQQLDEYQELLDIKLALDMEIHAYR | SEQ ID NO: 382 |
| NP_001909.3 | NVQICSIFDIATELNRLQKLGSVGQLSTTDLTGGTFTLSNIGSIGGTFA | SEQ ID NO: 383 |
| NP_001909.3 | AKPVIMPPEVAIGALGSIKAIPRFNQKGEVYKAQIMNVSWSADHRVIDG | SEQ ID NO: 384 |
| NP_056036.1 | ERVHRYISDQLFTNFKERTERLVISPLNQLLSMFTGPHKLVQKRFDKLL | SEQ ID NO: 385 |
| NP_056036.1 | GDVITIIGVPEPGWFEGELEGRRGIFPEGFVELLGPLRTVDESVSSGNQ | SEQ ID NO: 386 |
| NP_060225.4 | YPEELAWHTNLSRKILRKSPHLEKFHQFLVSETESGNISRQEAVSMIPP | SEQ ID NO: 387 |
| NP_060225.4 | DNKRCYLLVHQAKRLSSPCIMVVNHDASSIPRLQIDVDGRKEILFYDRI | SEQ ID NO: 388 |
| NP_054733.2 | GSFRRQRKGYEEVHVPALKPKPFGSEEQLLPVEKLPKYAQAGFEGFKTL | SEQ ID NO: 389 |
| NP_054733.2 | GISHDDLKGDPLLDQRRLDLVHTAALMLDKNNLVKYDKKTGNFQVTELG | SEQ ID NO: 390 |
| NP_054733.2 | NVFVGAPTGSGKTICAEFAILRMLLQSSEGRCVYITPMEALAEQVYMDW | SEQ ID NO: 391 |
| NP_006862.2 | NWSCRTPEPNPVTGRPLVNIYNCSGVQVGDNNYLTMQQTTALPTWGLAP | SEQ ID NO: 392 |
| NP_976037.1 | DKVKQKAFVEPYFKGDESIMQICQQKKEKRNWLPAQDTDSATFHPTHQR | SEQ ID NO: 393 |
| NP_001258900.1 | VITKHNDDEQYAWESSAGGSFTVRADHGEPIGRGTKVILHLKEDQTEYL | SEQ ID NO: 394 |
| NP_001258901.1 | TSLSEYVSRMKETQKSIYYITGESKEQVANSAFVERVRKRGFEVVYMTE | SEQ ID NO: 395 |
| NP_001176.1 | EAEPVYVQRAKAYLEEECPATLRKYLKYSKNILDRQDPPSVVVTSHQAP | SEQ ID NO: 396 |
| NP_001190.1 | GFWRKAPLRGRFCGSKLPEPIVSTDSRLWVEFRSSSNWVGKGFFAVYEA | SEQ ID NO: 397 |
| NP_001190.1 | PSKVCIWRIQVSEGFHVGLTFQSFEIERHDSCAYDYLEVRDGHSESSTL | SEQ ID NO: 398 |
| NP_001950.1 | GLVWGSSKLVPVGYGIKKLQIQCVVEDDKVGTDMLEEQITAFEDYVQSM | SEQ ID NO: 399 |
| NP_002466.1 | EPPVDLSKVVIEFNKDQLEEFKEAFELFDRVGDGKILYSQCGDVMRALG | SEQ ID NO: 400 |
| NP_002885.1 | HLEPLKSLGGRTSKRKKTEEESEHEVSCPQASFDKENAFPFPMDNQFSM | SEQ ID NO: 401 |
| NP_002889.1 | VKLCQPYGKIVSTKAILDKTTNKCKGYGFVDFDSPSAAQKAVTALKASG | SEQ ID NO: 402 |
| NP_000333.1 | SLIFIYETFSKLIKIFQDHPLQKTYNYNVLMVPKPQGPLPNTALLSLVL | SEQ ID NO: 403 |
| NP_995586.1 | PDCSPCEERLNPADIKRASQLASKMHSLLALMVGLLTCQDIINSDAEHF | SEQ ID NO: 404 |
| NP_004119.1 | MAERGELDLTGAKQNTGVWLVKVPKYLSQQWAKASGRGEVGKLRIAKTQ | SEQ ID NO: 405 |
| NP_005558.1 | IDITLSSVKCFHKLASAYGARQLQGYCASLFAILLPQDPSFQMPLDLYA | SEQ ID NO: 406 |
| NP_001001925.1 | LRETVSYCLIDDECPLMVPAFDKSEAQVLNPEHKVTETEDTQMVSKGKD | SEQ ID NO: 407 |
| NP_001001925.1 | DALPPEKTLELTQYKTKCENQSGFILQLKQLLACGNTKFEALTVVIQHL | SEQ ID NO: 408 |
| NP_001001925.1 | TGRRSGSTSSLSYSTWTSSHSDKTHARETTYDRESFENPQVTPSEAQDM | SEQ ID NO: 409 |
| XP_005270961.1 | GPGGVPVQGSKYAADRNHYRRYPRRRGPPRNYQQNYQNSESGEKNEGSE | SEQ ID NO: 410 |
| XP_005271379.1 | EIKEMLASDDEEDVSSKVEKAYVPKLTGTVKGRFAEMEKQRQEEQRKRT | SEQ ID NO: 411 |
| XP_005271380.1 | WWFEGEILQDGEDYQYIERGETYCLYLPETFPEDGGEYMCKAVNNKGSA | SEQ ID NO: 412 |
| XP_005273028.1 | SAAASPVHLKSPSLPAPSPGWTSSPKPPLQSPGIPPNHKAPLTMASPAM | SEQ ID NO: 413 |
| XP_005273028.1 | MLGNVESGGPPPPTASQPASVNIPGSLPSSTPYTMPPEPTLSQNPLSIM | SEQ ID NO: 414 |
| XP_005273028.1 | GSLPSSTPYTMPPEPTLSQNPLSIMMSRMSKFAMPSSTPLYHDAIKTVA | SEQ ID NO: 415 |
| XP_005273028.1 | AQKTPAKVVYVFSTEMANKAAEAVLKGQVETIVSFHIQNISNNKTERST | SEQ ID NO: 416 |
| XP_005246437.1 | MEVKPPPGRPQPDSGRRRRRRGEEGHDPKEPEQLRKLFIGGLSFETTDD | SEQ ID NO: 417 |
| XP_005264966.1 | SQVPAGERQTDRHVFETEPLQASGRPCGRRPVRYCSRVEIPSGQVSRQK | SEQ ID NO: 418 |
| XP_005264966.1 | MLFETRPLDRLGSRPSLQEQSPLELRSEIQELKGDVKKTVKLFQTEPLC | SEQ ID NO: 419 |
| XP_005247674.1 | KRLDIEEALVEEKKIVDNLKKEYDTLSKKVKIVATNLNAAEEALEAYQR | SEQ ID NO: 420 |
| XP_005262738.1 | ASSATSARQRRRQSKEQDEVRHGRDKGLINKENTPSGFNHLDDCILNTQ | SEQ ID NO: 421 |
| XP_005262738.1 | GSPSSKRRSPLLQPIIEGETASFFKEIKEEEEGSEDDSNVKPDFMVTLK | SEQ ID NO: 422 |
| XP_005263213.1 | TRLPDRSVAWPCPDRHLREKPSLEKVTFQIGSFASPESDFESRMKKMEE | SEQ ID NO: 423 |
| XP_005263215.1 | MAPTLLQKLFNKRGSSGSSAAASAQGRAPKEGPAFSWSCSEFDLNEIRL | SEQ ID NO: 424 |
| XP_005263216.1 | FAALLTAVLTYHLAWVPTVMPVDHPPIKAFSEKRTSQSVNMLAKTHPYN | SEQ ID NO: 425 |
| XP_005248433.1 | TIEQLDSLRDQFLDMAPKGIIGNKAFTDILIDLVTLNLGTNNFPSNWMH | SEQ ID NO: 426 |
| XP_005250082.1 | FMGQVVTGTQNSEGQNLGPQAIPQDGSITHQISRPNPPNFGPGFVNDSQ | SEQ ID NO: 427 |
| XP_005250082.1 | DGWSEQLPDTLVDESVSVTESTEKIKKRYRKRKNKLEETFPAYLQEAFF | SEQ ID NO: 428 |
| XP_005250088.1 | PVACVRKKSEMLQLFPAYLKGEDLFGLTVSAVARIAESLPGVEACENYT | SEQ ID NO: 429 |
| XP_005270318.1 | TKVTPGSELPTPNGTTLSSNLTGGMPFIDVPTPISSASSEAASAVVSPS | SEQ ID NO: 430 |
| XP_005270318.1 | LSLDALLTVIDSTEAHCQAKVLNSLTQQEKKETARPSCEIVDGTREASN | SEQ ID NO: 431 |
| XP_005270318.1 | AAKTVFHLAHRHGDILREGWKNIMEAMLQLFRAQLLPKAMIEVEDFVDP | SEQ ID NO: 432 |
| XP_005274302.1 | GDLKGPKVQANLGAPDINIEGLDAKVKTPSFGISAPQVSIPDVNVNLKG | SEQ ID NO: 433 |
| XP_005268896.1 | LDDGPYEAGGEKEQDPRLIPVPANSYFGLLFVTNIDSSDPDQLVYKTLD | SEQ ID NO: 434 |
| XP_005268896.1 | ASGTVWLKHQHDRVCGDAMFQLQENVKDKLRAIVVTLSYSLQTPRLRRQ | SEQ ID NO: 435 |
| XP_005269116.1 | TEPSGSGRTLRAWGLFFPVQQVTVGFPSPSLLNRNEFLEILMLPDSRGL | SEQ ID NO: 436 |
| XP_005257841.1 | CCHLKLPIPDQQLYDPVLVCNSCYEHIQVSRARELMSQQLKKPIATASS | SEQ ID NO: 437 |
| XP_005258382.1 | SATFHPTHQRIFGKLVFLPLRLVWKEVILRKILILVLVQKDVSLTTQYF | SEQ ID NO: 438 |
| XP_005259338.1 | RTINEVENQILTRDAKGISQEQMQEFRASFNHFDKKQTGSMDSDDFRAL | SEQ ID NO: 439 |
| XP_005262547.1 | LHADWTELTSCVPSVVDGKRDARLRPSRWRIFQILDAASKDKRGSPNTN | SEQ ID NO: 440 |
| XP_005262547.1 | SSVEINEEIQKRIEGLNFLDMELYSYAKDLFLQRYQFMRQKEHQEARRK | SEQ ID NO: 441 |
| XP_005258164.1 | TEEIKVLFIPSSPGVFRCTFSVASWPCSTDAETIVQAEALASTVTLTAI | SEQ ID NO: 442 |
| NP_000480.3 | LRTETQNALKEEEERRKRIAEREREREKLREVIEIEDASPTKCPITTKL | SEQ ID NO: 443 |
| NP_612114.2 | EEEEEEEEEEDENDDSKSPGKGRKKIRKILKDDKLRTETQNALKEEEER | SEQ ID NO: 444 |
| NP_775871.2 | MKLILWYLVVALWCFFKDVEALLYRQKSDGKIAASRSGGFSYGSSSSGD | SEQ ID NO: 445 |
| NP_775871.2 | ITGLSAGVTGTTGPSAGVTGSTGLLAGVTETTGQSAKVTGTTGQSVGVT | SEQ ID NO: 446 |
| NP_775871.2 | EPSAGLTETTGSSTGVTGATGPLAGVTGTTGISTEVTGTTGPSARVTGT | SEQ ID NO: 447 |
| NP_775871.2 | VIGTIGSSAVVTGINGLSAGVTGTTGPSAEETGATGPSAEVTGTTGPSA | SEQ ID NO: 448 |
| NP_775871.2 | ISAEVTGTTGPSAEVTGLPGVSAGVTGTIGSPAAVTGTTRPSAVVTGIS | SEQ ID NO: 449 |
| NP_775871.2 | VSAGLIGTIGSPAAVRGTTWPSAVVTGISGLSGEVTGTTGLSAGVTGIG | SEQ ID NO: 450 |
| NP_775871.2 | VTRTSEQSSGVTVASEPSVGVSGTTGPLAEISGTTRPLVSGLRTTGSSA | SEQ ID NO: 451 |
| NP_001269553.1 | KRKLESTESRSSFSQHARTSGRVAVEEVDEEGKFVRLRNKSNEDQSMGN | SEQ ID NO: 452 |
| NP_001269553.1 | EAKKQLQDEMLRRVDAENRLQTMKEELDFQKNIYSEELRETKRRHETRL | SEQ ID NO: 453 |
| NP_006120.1 | KAHFFSDKDECSKDNGGCQQDCVNTFGSYECQCRSGFVLHDNKHDCKEA | SEQ ID NO: 454 |
| NP_066933.1 | GLRGTETTLALPMASDLLLYDVTENSMRVKWDAVPGASGYLILYAPLTE | SEQ ID NO: 455 |
| NP_066933.1 | LPNTEYTVTVYAMFGEEASDPVTGQETTLALSPPRNLRISNVGSNSARL | SEQ ID NO: 456 |
| NP_004548.3 | NTAECGWDGGDCRPEDGDPEWGPSLALLVVLSPPALDQQLFALARVLSL | SEQ ID NO: 457 |
| NP_004548.3 | VGQAEETGPPSTCQLWSLSGGCGALPQAAMLTPPQESEMEAPDLDTRGP | SEQ ID NO: 458 |
| NP_004548.3 | LPQAAMLTPPQESEMEAPDLDTRGPDGVTPLMSAVCCGEVQSGTFQGAW | SEQ ID NO: 459 |
| NP_004548.3 | WLGCPEPWEPLLDGGACPQAHTVGTGETPLHLAARFSRPTAARRLLEAG | SEQ ID NO: 460 |
| NP_004548.3 | GGACLQARTWSVDLAARGGGAYSHCRSLSGVGAGGGPTPRGRRFSAGMR | SEQ ID NO: 461 |
| NP_003557.2 | IQAGEGETAVLNQLQEKNHTLQEQVTQLTEKLKNQSESHKQAQENLHDQ | SEQ ID NO: 462 |
| NP_003557.2 | AEKEKISVLQNNYEKSQETFKQLQSDFYGRESELLATRQDLKSVEEKLS | SEQ ID NO: 463 |
| NP_003557.2 | IEEIKCRQEKEITKLNEELKSHKLESIKEITNLKDAKQLLIQQKLELQG | SEQ ID NO: 464 |
| NP_003557.2 | ERCLKGEGEIEKLQTKVLELQRKLDNTTAAVQELGRENQSLQIKHTQAL | SEQ ID NO: 465 |
| XP_006710413.1 | MITEIIGSHLLPKANPPGASQQTWASGDNIPETEGLQASLSFLRKDSLP | SEQ ID NO: 466 |
| XP_006710446.1 | MWIRNGKNSFQVHSGCCTEDRFKEEKERKQGEQLRDDCCNSGSEKSQGM | SEQ ID NO: 467 |
| XP_006710446.1 | GCLFTWKVFDKDGRGIKREREVGFAEEVLVTKTDMEEKAQVMLELKTRV | SEQ ID NO: 468 |
| XP_006710446.1 | RLLKEKVRGLFEKYVQRADMVEIAGLNTDLQQEYTRQREHLERNLATLK | SEQ ID NO: 469 |
| XP_006712124.1 | LRIARAHGHLQSATDTWENLVSDARIKQGLIHQHCQVRIDTLGLLCESN | SEQ ID NO: 470 |
| XP_006712124.1 | ALALALAVLCDLLQQWDQLAPGLPILLGWLLGESDDLVACVESMHQVEE | SEQ ID NO: 471 |
| XP_006712126.1 | FGVKRAKDEHSKTNRMTGREFFSRFPELYPFLLKQLETVANTVDSDMGE | SEQ ID NO: 472 |
| XP_006712126.1 | LESLGFWEEVRGIISGSELITGFPWAFKVPGLPQYLQSLTRLAIAAVWA | SEQ ID NO: 473 |
| XP_006712128.1 | EHSKTNRCGHSPVYHSREMAARALVPFVMIDHIPNTIRTLLSTLPSCTD | SEQ ID NO: 474 |
| XP_006713760.1 | LAQDANGAIWKLDLSFSNITQDPECLFSFHSGAIEAVAVSPLTYLMATT | SEQ ID NO: 475 |
| XP_006713806.1 | MILTQRSVALGQDKVALQKLNDAATKLQACWRGFYARNYNPQAKDVRYE | SEQ ID NO: 476 |
| XP_006713806.1 | ACFPVQLDTLSDGASVDESHGISPPLQGEISQTQENSKLNAEVQGQQPE | SEQ ID NO: 477 |
| XP_006714120.1 | WEEPIPAFSSWQRENSDSDEAHLSPQAGRLIRQLLDEDSDPMLSPRFYA | SEQ ID NO: 478 |
| XP_006717798.1 | HQYAQGLFTNCVHGYAEAHCDFVHQALEQLKPLLSLLKVAGREGNLIAI | SEQ ID NO: 479 |
| XP_006718110.1 | QPLASPLQVGVPPMTLPIILNPALIEATSPVPLLATPRPTDPIPTSEVN | SEQ ID NO: 480 |
| XP_006718114.1 | VASSPSRLSPTPDGPPPLAQPPLILQPLASPLQVGVPPMTLPIILNPAL | SEQ ID NO: 481 |
| XP_006718732.1 | LAARFDRKAAMRETWLSENQRLVSQDNFGLELAAVEAAVRKHEAIETDI | SEQ ID NO: 482 |
| XP_006718732.1 | LRATMRRREESLGEARRLQDFLRSLDDFQAWLGRTQTAVASEEGPATLP | SEQ ID NO: 483 |
| XP_006718732.1 | AAQQFLGRLRDNREQQHFLQDCHELKLWIDEKMLTAQDVSYDEARNLHT | SEQ ID NO: 484 |
| XP_006718732.1 | DVSSADLVIKNQQGIKAEIEARADRFSSCIDMGKELLARSHYAAEEISE | SEQ ID NO: 485 |
| XP_006718732.1 | EKERKRKREEEERRKQPPAPEPTASVPPGDLVGGQTASDTTWDGTQPRP | SEQ ID NO: 486 |
| XP_006719604.1 | TEPSGSGRTLRAWGLFFPVQQVTVGLGLEKEISTFLPFTIADGANGNHH | SEQ ID NO: 487 |
| XP_006722928.1 | KEDKYEEEIKLLSDKLKEAETRAEFAERTVAKLEKTIDDLEDELYAQKL | SEQ ID NO: 488 |
| XP_006723899.1 | TLRVDTYTLVQPEAEDHVESYRTMPIYPTYNEVHLDERPFLRPNIISGK | SEQ ID NO: 489 |
| XP_006723899.1 | MGRNPHQGRRNQEGHASDEARDQRHDQENDTRWRNGNQDCRNRRPPWSN | SEQ ID NO: 490 |
| XP_006723899.1 | GCGHVCTRACHPYDSSHKEFQCMKPCQKVICQEGHRCPLVCFQECQPCQ | SEQ ID NO: 491 |
| XP_006723899.1 | CKRLLICSHKCQEPCIGECPPCQRTCQNRCVHSQCKKKCGELCSPCVEP | SEQ ID NO: 492 |
| XP_005262211.2 | DQNSDSDEMLAILKEVSRMSHSSSSDTDINEIHTNHKTLYDLKTQAGKD | SEQ ID NO: 493 |
| XP_005262214.2 | VIKLKPHQVDGVQFMWDCCCESVKKTKKSPGSGCILAHCMGLGKTLQVV | SEQ ID NO: 494 |
| NP_001276916.1 | PIQIMTVSTSAAPAEIAVSPESQEMPMGRTILKVTPEKQTVPTPVRKYN | SEQ ID NO: 495 |
| NP_001276916.1 | NSNANIITTEDNKIHIHLGSQFKRSPGTSGEGVSPVITVRPVNVTAEKE | SEQ ID NO: 496 |
| NP_001276916.1 | LEQLLLAEKCHRRTVYELENEKHKHTDYMNKSDDFTNLLEQERERLKKL | SEQ ID NO: 497 |
| NP_001276916.1 | HTDYMNKSDDFTNLLEQERERLKKLLEQEKAYQARKEKENAKRLNKLRD | SEQ ID NO: 498 |
| NP_001277033.1 | IKLVLDDCWATSTMDPDSFPQWNVVVDGCAYDLDNYQTTFHPVGSSVTH | SEQ ID NO: 499 |
| NP_008877.1 | IQGHEPRIADLRERQRALGAAAAGPELAELQEMWKRLGHELELRGKRLE | SEQ ID NO: 500 |
| NP_008877.1 | ELAGERRERLQEHLRLCQLRRELDDLEQWIQEREVVAASHELGQDYEHV | SEQ ID NO: 501 |
| NP_009187.1 | DNTSPEDMPQDFKASLCLPTTSAPASAPSNGNCSSYTETQHLSVVGIMF | SEQ ID NO: 502 |
| NP_683515.4 | LNVQLEYSRSHLKKKLNKINTLKETIQKGSEDIDHLKKAEENCLQTVNE | SEQ ID NO: 503 |
| NP_001611.1 | HFKSPQISMSDIDLNLKGPKIKGDMDISVPKLEGDLKGPKVDVKGPKVG | SEQ ID NO: 504 |
| NP_001013764.1 | QGQKLYFAHHPAFAHFETIDLSQATVAESSCRNLCHSFCVITPQRKITL | SEQ ID NO: 505 |
| NP_001013764.1 | LMGSVNASANILNDIFYGQDSGNEMGAASCIPIETLSRNDAVDVTFSLK | SEQ ID NO: 506 |
| NP_001017995.1 | RPNFLAPLPHEVTQLRLGEAAALENNTGSEATGPSRPLPDAPHGVMDSG | SEQ ID NO: 507 |
| NP_001017995.1 | IIKSEGELLERERERQRTEQLRGPTPKPPGVILPMMPAKHIPPARDSRR | SEQ ID NO: 508 |
| NP_001280736.1 | AVAGSQLTATSAARNLVLCLLFLLGRSALKRCLCPGEPPQEGASLEGRG | SEQ ID NO: 509 |
| NP_001287795.1 | MRSRNQGGESASDGHISCPKPSIIGNAGEKSLSEDAKKKKKSNRKEDDV | SEQ ID NO: 510 |
| NP_001287795.1 | SELEKLEEAFSKSKSECTQLHLNLEKEKNLTKDLLNELEVVKSRVKELE | SEQ ID NO: 511 |
| NP_003465.3 | ACRDSSNSCDLPEFCTGASPHCPANVYLHDGHSCQDVDGYCYNGICQTH | SEQ ID NO: 512 |
| NP_003465.3 | DSGPIRQADNQGLTIGILVTILCLLAAGFVVYLKRKTLIRLLFTNKKTT | SEQ ID NO: 513 |
| NP_001290330.1 | LSPLVPVETRASLIPEHSSPVQDCQISQESESTFMPVASGLSPLSPTVE | SEQ ID NO: 514 |
| NP_001028725.1 | TTQEGKIIELEQKCTGILEQGRFEREKLLNIQQQLTCSLRKVEEENQGA | SEQ ID NO: 515 |
| NP_001028725.1 | QVRELLAEAKAKDSEINRLRSELKKYKEKRTLNAEGTDALGPNVDGTSV | SEQ ID NO: 516 |
| NP_001028727.1 | TSRAAPPPVDEEPESSEVDAAGRWPGVCVSRTSPTPPESATTVKSLIKS | SEQ ID NO: 517 |
| NP_001028727.1 | SFDLGRPGGAGQNISVHKTPRSPLSGIPVRTAPAAAVSPMQRHSTYSSV | SEQ ID NO: 518 |
| NP_001028727.1 | DEYKKNIHGNALRTSGSSSSDVTKASLSPDASDFEHITAETPSRPLSST | SEQ ID NO: 519 |
| NP_055689.1 | NLEQEIAAMNKKIEVLDSLQSKAKLLRNKAGWLDSELNMFTHQYLQPSR | SEQ ID NO: 520 |
| NP_053585.1 | KMKSQTGQVGEKQSLKCEAAAGNPQPSYRWFKDGKELNRSRDIRIKYGN | SEQ ID NO: 521 |
| NP_053585.1 | SWSGHARKCNETAKSYCVNGGVCYYIEGINQLSCKCPNGFFGQRCLEKL | SEQ ID NO: 522 |
| XP_006726566.2 | WARALSPAQLHRARACAPPSEGLLFRWDPGALDVTPSLLPTVWVRLLCP | SEQ ID NO: 523 |
| XP_006726566.2 | APLGPAALLAVVRFLKRVVALGAGDPELLLTGPWEQLSQGVVSVASLVL | SEQ ID NO: 524 |
| XP_011508532.1 | HELFRQCTKNEVTKEMKTNAMGLIRSLENIFAESKIKSFRRQVQEENFE | SEQ ID NO: 525 |
| XP_011508535.1 | FEQAEKRKLQETKELQKAGIMFQMDNHLPNLVNLNEDPQLSEMLLYMIK | SEQ ID NO: 526 |
| XP_011508535.1 | PVSSLSRRRSRSLMKNRRISGCLHDIQVHPIKNLHSSHSSGLMDKSSTI | SEQ ID NO: 527 |
| XP_011508537.1 | NDDPLLRSADEDPQVIEMMADKKYKETFSAPSRANENVALKYSSNRPPI | SEQ ID NO: 528 |
| XP_011509143.1 | VRLIIRHTHIPRLIPVITSNCTSKSVAVRRRCFEFLDLLLQEWQTHSLE | SEQ ID NO: 529 |
| XP_011509143.1 | DWEQRVNALKKIRSLLLAGAAEYDNFFQHLRLLDGAFKLSAKDLRSQVV | SEQ ID NO: 530 |
| XP_011509143.1 | PRSQGCSRETSPNRIGLDRFGLGQPGRIPGSVNAMRVLSTSTDLEAAVA | SEQ ID NO: 531 |
| XP_011509143.1 | AIYSVIGEDLKPHLAQLTGSKMKLLNLYIKRAQTTNSNSSSSSDVSTHS | SEQ ID NO: 532 |
| XP_011509149.1 | FDDDMEQLRDVPIDHSDLVADLLKELSNHNERVEERKGALLELLKITRE | SEQ ID NO: 533 |
| XP_011509520.1 | TDTEMVEKVFGFLPAMIGGQEGQASPHFEDLESKTQKLLEVDLDTVPMA | SEQ ID NO: 534 |
| XP_011509520.1 | QKSASHTHIRRPLRYPLLYHEDDTDCLAALVIWNVILRFMGDLPEPVLY | SEQ ID NO: 535 |
| XP_011509520.1 | SMAVLPLARARGHLWAYSCEPLRQPLLKRVHANVDLWDIACQIFVAILR | SEQ ID NO: 536 |
| XP_011509972.1 | DPNIPDEYGNTTLHYAIYNEDKLMAKALLLYGADIESKNKHGLTPLLLG | SEQ ID NO: 537 |
| XP_011509972.1 | YHSDEQNDTQKQFCEEQNTGILHDEILIHEEKQIEVVEKMNSELSLSCK | SEQ ID NO: 538 |
| XP_011509972.1 | LSGGTTMYPGMAHRMQKEIAALAPSMLKIRIIAPPKRKYSVWVGGSILA | SEQ ID NO: 539 |
| XP_011510516.1 | SMAQPMASSSTEPYLCLAAAGENPSGKSLASAISGKIPSPLSSSYKPML | SEQ ID NO: 540 |
| XP_011531663.1 | CPFQSWPEAYGSDFWKSVHFTDYSQHTQMVMALTLRCPLKLEAALCQRH | SEQ ID NO: 541 |
| XP_011531664.1 | TGSSAYIPCRTWWALFSTKPWCVRVWHCSRCLCQHLLSGGSGLQRGLFH | SEQ ID NO: 542 |
| XP_011511210.1 | DLSNPGGRPALAYESVVAQEGSPILRDLVLSPNHQYLYAMTEKQVTRVP | SEQ ID NO: 543 |
| XP_011511210.1 | DLLQCVQLTVQPRNVSVTMSQVPLVLQAWNVPDLSAGVNCSFEDFTESE | SEQ ID NO: 544 |
| XP_011511210.1 | PQNIQAHLYKCPALRESCGLCLKADPRFECGWCVAERRCSLRHHCAADT | SEQ ID NO: 545 |
| XP_011511210.1 | GTLQVYSDSLLTLPAIVGIGGGGGLLLLVIVAVLIAYKRKSRDADRTLK | SEQ ID NO: 546 |
| XP_011529980.1 | DVLLDVTQSPGDGSGLMLEEVTGLKCNFESAREGNEQLTEEERLLKFSI | SEQ ID NO: 547 |
| XP_011529980.1 | EVGAYVSVINGPEIISKFYGETEAKLRQIFAEATLRHPSIIFIDELDAL | SEQ ID NO: 548 |
| XP_011530277.1 | QPPPPPPPPQSPEEGTTYIAPAKEPSVNTALVPQLSTISRALTPSPVMV | SEQ ID NO: 549 |
| XP_011530277.1 | RLQLTFEEYTIMKVLLLLSTIPKDGLKSQAAFEEMRTNYIKELRKMVTK | SEQ ID NO: 550 |
| XP_011530277.1 | KGNGHRPSTLSCVNTPLRSFMSDSGSSVNGGVMRAVVKSPIMCHEKSPS | SEQ ID NO: 551 |
| XP_011530277.1 | GSSVNGGVMRAVVKSPIMCHEKSPSVCSPLNMTSSVCSPAGINSVSSTT | SEQ ID NO: 552 |
| XP_011512325.1 | KLQAKSPKEAEADLSFDSYHSTQTDLGPSLGKPGETSPPDSKSSPSVLI | SEQ ID NO: 553 |
| XP_011512325.1 | ALNSLSQLSYSTSSSKRQSQQLEALQQQVKQLQNQLAECKKQHQEVISV | SEQ ID NO: 554 |
| XP_011512437.1 | HGLQAALQNFLKGYPAIVRQLHSAGRGKSDTSQKKPKELLESLLQADIV | SEQ ID NO: 555 |
| XP_011535847.1 | TISLSSCRRDDNKDIDSEKEAAMEAEIKAARERAIVPLEARMKQFKDML | SEQ ID NO: 556 |
| XP_011535850.1 | RTLRKDHRWESGSLLEREEKEKLFNEHIEALTKKKREHFRQLLDETSAI | SEQ ID NO: 557 |
| XP_011536012.1 | PFLGLRGAHDALRSDSPPLCPAADSRTYYSLDSHSTRASSRHSRGPPPR | SEQ ID NO: 558 |
| XP_011532914.1 | EQYDVPKGSKNRKCIPGSIKLDSEEDMPFEDCTNDPESEHDLLINGCLK | SEQ ID NO: 559 |
| XP_011532914.1 | ALISKCSREKNKPQRSLVCGSKVKLCYIGAGDEEKRSDSISICTTSDDG | SEQ ID NO: 560 |
| XP_011532914.1 | LPASGKSRSDCVTRRNCGRSKPSSKLRDAFSAQMVKNTVNRKALKTERK | SEQ ID NO: 561 |
| XP_011532914.1 | KVKNDDSSKEIPGSEGELMPHRTATSPKETVEEGVEHDPGMPASKKMQG | SEQ ID NO: 562 |
| XP_011532914.1 | RCLLPLCGKFYHEECVQKYPPTVMQNKGFRCSLHICITCHAANPANVSA | SEQ ID NO: 563 |
| XP_005266969.2 | DTDDEELRHRIRQKQEAFWRKEKEQQLLHDKQMEEEKQQTERVTKEMNE | SEQ ID NO: 564 |
| XP_005266969.2 | VKQEPPQIDAVKRRTLPAWIREGLEKMEREKQKKLEKERMEQQRSQLSK | SEQ ID NO: 565 |
| XP_011514753.1 | VDMPRPLNNSQMNNPVGLPQHFSPQSLPVQQHNILGQAYIELRHRAPDG | SEQ ID NO: 566 |
| XP_011514754.1 | ITLHPTAAENISSVVAAFSDLLHVRIPNSYEVSSAPDVPSMGLVSSHRI | SEQ ID NO: 567 |
| XP_011514754.1 | QLLYKTNMNTGFLDPSLDPLLSSSSAPTKSGTHDIGPVTDDPSSLPQPN | SEQ ID NO: 568 |
| XP_011514754.1 | SAPTKSGTHDIGPVTDDPSSLPQPNVNQSSRPLSEEQLDGILSPELDKM | SEQ ID NO: 569 |
| XP_011514754.1 | NVNQSSRPLSEEQLDGILSPELDKMVTDGAILGKLYKIPELGGKDVEDL | SEQ ID NO: 570 |
| XP_011514754.1 | SRPTSQDPYSQPPGTPRPVVDSYSQSSGTARSNTDPYSQPPGTPRPTTV | SEQ ID NO: 571 |
| XP_011514916.1 | MCSSKSESDISKIADSRDDLRMFDSSGNNPVLSATINNLRMPVSQKITE | SEQ ID NO: 572 |
| XP_011542890.1 | LLPLENPKDTCASKSESKIDLTVSSDHFSGQQENLNSMKKRNVNFSAAE | SEQ ID NO: 573 |
| XP_011542890.1 | FPKPKDIPQAKEMFIDTVISSYNIETAHDSSNCSITREHICVHRKNENE | SEQ ID NO: 574 |
| XP_011542890.1 | TGRSVEHLASTTFPKTASSSVCVASNAAIQIASATMPALSLNNDDHQIY | SEQ ID NO: 575 |
| XP_011542890.1 | CSANHTALIANLSQILQRADEASSLQILQEETKVCLNILPLFVEAFERK | SEQ ID NO: 576 |
| XP_011515869.1 | DPQTSREIFNFVLKAIRPQIDLKRYAVPSAGLRLFALHASQFSTCLLDN | SEQ ID NO: 577 |
| XP_011515869.1 | RHRDDERQKVCLDIIYKMMPKLKPVELRELLNPVVEFVSHPSTTCREQM | SEQ ID NO: 578 |
| XP_011515869.1 | IIANALSSEPACLAEIEEDKARRILELSGSSSEDSEKVIAGLYQRAFQH | SEQ ID NO: 579 |
| XP_011515869.1 | VRMSTSPEAFLALRSHFASSHALICISHWILGIGDRHLNNFMVAMETGG | SEQ ID NO: 580 |
| XP_011515869.1 | EKRLSFAVPFREMKPVIFLDVFLPRVTELALTASDRQTKVAACELLHSM | SEQ ID NO: 581 |
| XP_011515869.1 | ERTVGALQVLGTEAQSSLLKAVAFFLESIAMHDIIAAEKCFGTGAAGNR | SEQ ID NO: 582 |
| XP_011516544.1 | QSPEPDLHKEALVRSTLSFWSQSKFGGLEGLKDNGSPIFHGRIIPKEAQ | SEQ ID NO: 583 |
| XP_011537862.1 | RAEKLKDKKTLEELQSARNNYEALNAQLLDELPKFHQYAQGLFTNCVHG | SEQ ID NO: 584 |
| XP_011537862.1 | ANLGQPQLWYIDHENVNHNLEDCWATNFYCREHSNLYHVPGSYPDGQPV | SEQ ID NO: 585 |
| XP_011538616.1 | SCLLPDPRELIEIKNKKKLLITGTEQFNQKPKKGIQFLQEKGLLTIPMD | SEQ ID NO: 586 |
| XP_011518459.1 | KVKQLIRLIHGIDLSKPVDSEATVGAISNGPDCTPPANAATSTPAPSPS | SEQ ID NO: 587 |
| XP_011543226.1 | YYLATDPMSGAVFLSDSNSRRVFKIKSTVVVKDLVKNSEVVAGTGDQCL | SEQ ID NO: 588 |
| XP_011543226.1 | LEKSMVLLLHSQRQYIFEFDKNDRLSSVTMPNVARQTLETIRSVGYYRN | SEQ ID NO: 589 |
| XP_011543231.1 | LGFLGPDCGRASCPVLCSGNGQYMKGRCLCHSGWKGAECDVPTNQCIDV | SEQ ID NO: 590 |
| XP_011543231.1 | METACGDSKDNDGDGLVDCMDPDCCLQPLCHINPLCLGSPNPLDIIQET | SEQ ID NO: 591 |
| XP_011543231.1 | RHEENEIPSCDLSNFARPNPVVSPSPLTSFASSCAEKGPIVPEIQALQE | SEQ ID NO: 592 |
| XP_011543231.1 | ENHQVRIVAGRPMHCQVPGIDHFLLSKVAIHATLESATALAVSHNGVLY | SEQ ID NO: 593 |
| XP_011543232.1 | YEITEDTASSWPVPTDVSLYPSGGTGLETPDRKGKGTTEGKPSSFFPED | SEQ ID NO: 594 |
| XP_011543551.1 | EGDFKGPKVDIKAPEVNLNAPDVDVHGPDWNLKMPKMKMPKFSVSGLKA | SEQ ID NO: 595 |
| XP_011543551.1 | NIKAPKISMPDIDLNLKGPKVKGDVDISLPKLEGDLKGPEVDIKGPKVD | SEQ ID NO: 596 |
| XP_011543551.1 | PKMKMPKFSMPGFKAEGPEVDVNLPKADVVVSGPKVDVEVPDVSLEGPE | SEQ ID NO: 597 |
| XP_011543552.1 | GSLEGEAEAEASSPKGKFSLFKSKKPRHRSNSFSDEREFSGPSTPTGTL | SEQ ID NO: 598 |
| XP_011537116.1 | NESKEKVSQLDIQIKAKTELLLSAEAAKTAQRADLQNHLDTAQNALQDK | SEQ ID NO: 599 |
| XP_011533406.1 | EWLKVYQEASTILKDRAQRLEECYEIIEKNLLLLGATAIEDRLQAGVPE | SEQ ID NO: 600 |
| XP_011535567.1 | NCHQGIGKGKRKEEAYKSENPKKLKWLGEKTLKRPWSTSETFTAWCNSH | SEQ ID NO: 601 |
| XP_011523974.1 | GEPVQNSVAVGICLGSNIGSGWMGTSSLCNPYSNTLNQNLLSTTKPFPV | SEQ ID NO: 602 |
| XP_011523974.1 | TSSLCNPYSNTLNQNLLSTTKPFPVPSVGTNCGIEPWDSGVTSGLGSVR | SEQ ID NO: 603 |
| XP_011523975.1 | KESKIVFSVRNTGSRAAFVKAVGFKDSQKKVLLDPKVLRIFPDKFVLKE | SEQ ID NO: 604 |
| XP_011523977.1 | TSSSGCALESFGSAAQQQQPPCEQELSPLVCSPAGVSRLTYVSEPESSY | SEQ ID NO: 605 |
| XP_011523977.1 | IIQGSPAALEERAMEKLREKVPFQNRGKGTLSSIIQNNSDTRKATETTS | SEQ ID NO: 606 |
| XP_011523977.1 | DFGDLTYGGWKALPLKLINRTHATVPIRLIINANAVAWRCFTFSKESVR | SEQ ID NO: 607 |
| XP_011523977.1 | PQHGCVAPESKLQILVSPNSSLSTKQSMFPWSGLIYIHCDDGQKKIVKV | SEQ ID NO: 608 |
| XP_011526200.1 | SQSQLDEVAACPQYGAASRVRKRPHWGCVCFLWYILETWLHTPLHLLLQ | SEQ ID NO: 609 |
| XP_011525622.1 | LELVVSELEARVGEEEECSRQMQTEKKRLQQHIQELEAHLEAEEGARQK | SEQ ID NO: 610 |
| XP_011525622.1 | LRAQVTELEDELTAAEDAKLRLEVTVQALKTQHERDLQGRDEAGEERRR | SEQ ID NO: 611 |
| XP_011525624.1 | SLTTELSAERSFSAKAESGRQQLERQIQELRGRLGEEDAGARARHKMTI | SEQ ID NO: 612 |
| XP_011525780.1 | TGVRLLCKRLSHPGCKLRVLWLFGMDLNKMTHSRLAALRVTKPYLDIGC | SEQ ID NO: 613 |
| XP_011525782.1 | ANKNLTRMDLSGNGVGFPGMMLLCEGLRHPQCRLQMIQLRKCQLESGAC | SEQ ID NO: 614 |
| XP_011527330.1 | WVESWMTRPDLQPGYEGWQALDPTPQEKSEGTYCCGPVPVRAIKEGDLS | SEQ ID NO: 615 |
| XP_011528036.1 | GVSLRSLYKRSSGHVTFTMDPIRDLLIWAIVQNRRELAGIIWAQSQDCI | SEQ ID NO: 616 |
| XP_011528036.1 | GPNHTLYPMVTRWRRNEDGAICRKSIKKMLEVLVVKLPLSEHWALPGGS | SEQ ID NO: 617 |
| XP_011528039.1 | YDPPFYTAERKDAAAMDPMGENPMGRTGLRGRGSLSCFGPNHTLYPMVT | SEQ ID NO: 618 |
| XP_011528039.1 | VKLPLSEHWALPGGSREPGEMLPRKLKRILRQEHWPSFENLLKCGMEVY | SEQ ID NO: 619 |
| NP_001293018.1 | CTECSCVFKSLGSLNTHISKMHMGGPQNSTSSTETAHVLTATLFQTLPL | SEQ ID NO: 620 |
| NP_001293018.1 | SITVGINQDILQQALENSGLSSIPAAAHPNDSCHAKTSAPHAQNPDVSS | SEQ ID NO: 621 |
| NP_001008392.2 | QLLRQQIQANTEMEAELKVLKENNQTLERDNELQREKVKENEEKFLNLQ | SEQ ID NO: 622 |
| NP_001297064.1 | QASRLLTDKLSYKSSVITLHQHSRTNFWCFLPRKSASFESIQKWFSAQP | SEQ ID NO: 623 |
| NP_659419.3 | LLPYPRRRPPHSARGGGSGGGGGSSSSSSSSQQQLRNFSRSRHASERGH | SEQ ID NO: 624 |
| NP_659419.3 | AEQASKPKVPPKSEKENDPLRTPEALPEEKKIEYRLLKEEIANREKQRL | SEQ ID NO: 625 |
| NP_659419.3 | IALHQLLERYEAAMELCKSLLESCPINCQLLEALVALYLQTNQHDKARA | SEQ ID NO: 626 |
| NP_001035095.1 | PVKAKKRKRDDNKDIDSEKEAAMEAEIKAARERAIVPLEARMKQFKDML | SEQ ID NO: 627 |
| NP_001035095.1 | TYRSKKLIQESDQHLKDVEKILQNDKRYLVLDCVPEERRKLIVAYVDDL | SEQ ID NO: 628 |
| NP_689976.2 | SPSCAFCRRLLEWKQNVEKKGHGRINCGRFSIVLHEKAPHSDSTPNSDT | SEQ ID NO: 629 |
| NP_061833.1 | HMVTLDYTVQVPGAGQDGSPGLSKFRLSYYPHCLASFTELLQAAFGGKC | SEQ ID NO: 630 |
| NP_002461.2 | QELLDSNERVQLLHTQNTSLIHTKKKLETDLMQLQSEVEDASRDARNAE | SEQ ID NO: 631 |

*NCBI - National Center for Biotechnology Information (Bethesda, Maryland, USA)

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

1. A method of detecting an anti-IL1RA antibody in a biological sample, comprising:
(a) contacting the biological sample with an IL1RA polypeptide; and,
(b) detecting binding of the IL1RA polypeptide to the anti-IL1RA antibody in the biological sample.

2. A method of identifying a subject at risk of premature labor, comprising:
(a) contacting the biological sample obtained from the subject with an IL1RA polypeptide; and,
(b) detecting presence of binding of the IL1RA polypeptide to the anti-IL1RA antibody in the biological sample,
wherein the presence of binding of the IL1RA polypeptide to the anti-IL1RA antibody in the biological sample identifies the subject at risk of premature labor.

3. The method of claim 1, wherein the subject is a pregnant subject.

4. A method of detecting presence of an anti-IL1RA antibody in a pregnant subject and treating the pregnant subject, comprising:
(a) contacting a biological sample obtained from the pregnant subject with an IL1RA polypeptide;
(b) detecting presence of binding of the IL1RA polypeptide to the anti-IL1RA antibody in the biological sample; and,
(c) administering to the pregnant subject one or more treatments intended to prevent premature labor or to promote fetal maturation.

5-9. (canceled)

10. The method of claim 1, wherein the IL1RA polypeptide is a heterologously expressed IL1RA polypeptide.

11. The method of claim 10, wherein the heterologously expressed IL1RA polypeptide is expressed on the surface of a cell, on a phage, or on a virus.

12. The method of claim 10, wherein the heterologously expressed IL1RA polypeptide is expressed in a phage display or eukaryotic cell display library.

13. The method of claim 1, wherein the IL1RA polypeptide is an isolated polypeptide.

14. The method of claim 1, wherein the IL1RA polypeptide is immobilized on a solid carrier.

15. The method of claim 1, wherein step (b) comprises performing at least one of immunoprecipitation, microarray analysis, enzyme-linked immunosorbent assay (ELISA), or Western blot analysis.

16. The method of claim 1, wherein the IL1RA polypeptide comprises a sequence with at least 90% sequence similarity to SEQ ID NO:2.

17. The method of claim 1, wherein the biological sample is serum, plasma, blood, amniotic fluid, or urine.

18-19. (canceled)

20. The method of claim 2, wherein the subject previously experienced premature labor or premature delivery.

21. The method of claim 2, wherein the subject is a pregnant human.

22. The method of claim 1, wherein the anti-IL1RA antibody is an autoantibody.

23. The method of claim 1, wherein the anti-IL1RA antibody is an alloantibody.

24-56. (canceled)
